(19) United States
(12) Patent Application Publication
(10) Pub. No.: US 2021/0147929 A1
(43) Pub. Date: May 20, 2021

(54) IMMUNE RECEPTOR ANALYSIS AS DIAGNOSTIC ASSAY

(71) Applicant: Saint Louis University, St. Louis, MO (US)

(72) Inventors: Richard DiPaolo, High Ridge, MO (US); Kyle Wolf, St. Louis, MO (US); Tae Hyuk Ahn, Chesterfield, MO (US)

(73) Assignee: Saint Louis University, St. Louis, MO (US)

(21) Appl. No.: 17/068,546

(22) Filed: Oct. 12, 2020

Related U.S. Application Data
(60) Provisional application No. 62/914,169, filed on Oct. 11, 2019.

(51) Int. Cl. C12Q 1/6869; C12Q 1/6876; C12Q 1/686; C12Q 1/6853; G01N 33/50

(52) U.S. Cl. CPC C12Q 1/6869; C12Q 1/6876; C12Q 2525/173; C12Q 1/6853; G01N 33/5008; C12Q 1/686

(57) ABSTRACT

Methods of determining an immune status of a subject are provided. Specifically, methods for determining whether a subject has been exposed to an immunogenic antigen are provided as well as methods for determining efficacy of a vaccine are described.

### REFERENCE TO RELATED APPLICATIONS

This application claims the benefit of U.S. Provisional Application Ser. No. 62/914,169, filed Oct. 10, 2019, the contents of which are incorporated by reference herein.

### STATEMENT REGARDING FEDERALLY SPONSORED RESEARCH OR DEVELOPMENT

This invention was made with Government support under DJF-15-1200-P-0001007 awarded by Federal Bureau of Investigations. The Government has certain rights in the invention.

### REFERENCE TO SEQUENCE LISTING SUBMITTED ELECTRONICALLY

The official copy of the sequence listing is submitted electronically via EFS-Web as an ASCII-formatted sequence listing with a file named “SLU19007US GENE SEQUENCE LISTING,” created on Oct. 9, 2020, and having a size of 126 kilobytes, and is filed concurrently with the specification. The sequence listing contained in this ASCII-formatted document is part of the specification and is herein incorporated by reference in its entirety.

### FIELD OF THE INVENTION

The invention relates to methods for determining exposure to an immunogenic antigen or a viral infection in a subject. Also provided are methods for evaluating vaccine efficacy.

### BACKGROUND OF THE INVENTION

T cell and B cell responses are responsible for generating the adaptive immune response to vaccines and infections. T cells recognize pathogen-specific peptides in the context of the major histocompatibility complex (MHC) through the T cell receptor (TCR). The TCR is a heterodimeric protein composed of TCRα and TCDβ chains. During T cell development, each TCR chain is generated through quasi-random genetic recombination from the germline loci of the variable (V), diversity (D), and joining (J) gene segments (Manfras et al., 1989, Robins et al., 2010).

In mice, the tcrb locus has approximately 35 different TCRV β segments, 2 TCRD β segments, and 14 TCRJ β segments. During recombination, tcrv, tcrd, and tcrj segments are rearranged together to create and encode complementary determining region 3 (CDR3). CDR3 is the most variable region of the TCR that interacts with foreign peptide. These genetic rearrangement events result in a high degree of diversity in CDR3 of the TCR (Arstila et al., 1999, Cabaniols et al., 2001, Davis and Bjorkman, 1988, Robins et al., 2009).

During an immune response, antigen presentation results in the activation and expansion of T cells with TCR(s) specific to the pathogen (Ishizuka et al., 2009, Venturi et al., 2008b, Venturi et al., 2016). Clonally expanded T cells carry the same unique TCR rearrangement (Manfras et al., 1999). Once the pathogen has been cleared, a subset of T cells with TCRs specific to the pathogen remain as long-lived memory cells. The unique DNA rearrangements have the potential to serve as a stable biomarker, cataloging an individual's functional T cell memory and immunological history (Emerson and DeWitt, 2017, Estorninho et al., 2013).

On average, approximately 10⁷ unique TCRβ chains can be identified from the approximately 101 circulating T cells present in a healthy human adult (Robins et al., 2009). The ability to readily identify identical TCR sequences among multiple individuals (public TCRs) is challenging because an individual has the potential to generate approximately 10¹⁸ unique TCR recombinants. Nonetheless, in both humans and murine models, there are examples of public T cell responses to infectious disease (such as cytomegalovirus [CMV] and influenza) and in autoimmunity (Elhanati et al., 2014, Emerson and DeWitt, 2017, Li et al., 2012, Lossius et al., 2014, Marrero et al., 2016, Valkenburg et al., 2016, Venturi et al., 2008b). The presence of virus-specific public TCRs may be due partly to preferential use of specific TCR V and J chains in response to conserved hierarchy of epitope recognition (Chen et al., 2000, Hancock et al., 2015, Kim et al., 2013). Public TCR sequences from antigen-experienced T cells should be readily identifiable within the circulating T cell repertoire because of clonal expansion and the formation of memory T cell populations (Emerson and DeWitt, 2017, Heit et al., 2017).

Identifying antigen-specific T cells and tracking an antigen-specific response over time within individuals is a difficult task, especially against emerging pathogens, in which case precise immunogenic epitopes are not well described. Even when antigens are known, the frequencies of antigen-specific T cell populations are notoriously low and can often be difficult to identify (Douillard et al., 1997, Wolf and DiPaolo, 2016, Lim et al., 2000). This is due partly to a lack of knowledge concerning antigen-specific TCR sequences. Another issue is that antigen-specific TCR identification using many traditional immune assays is restricted to the most high-frequency responders (Wolf and DiPaolo, 2016, van der Velden et al., 2014, van der Velden and van Dongen, 2009). However, advancements in next-generation sequencing are allowing researchers to analyze TCR and B cell receptor (BCR) (Ig) repertoires (immunosequencing) with unprecedented depth and sensitivity, identifying 10⁵-10⁷ individual sequences in humans from a very limited volume of whole blood (DeWitt et al., 2015, Faham et al., 2012, Kirsch et al., 2015, Logan et al., 2014, Robins et al., 2009).

What is needed is a method to evaluate an exposure status of a subject to an immunogenic agent. This method ideally should be able to evaluate whether or not a subject has been exposed to the immunogenic agent and should be sensitive enough to accurately distinguish between closely related immunogenic agents. Further, a method for evaluating the effectiveness of vaccines (particularly their ability to generate a robust immune response) is needed.

### BRIEF SUMMARY OF THE INVENTION

Provided herein is a method for determining whether a subject has been exposed to an immunogenic antigen. The method comprises: amplifying and sequencing TCRβ alleles in mRNA and/or genomic DNA obtained from T-cells of the subject; identifying unique TCRβ alleles sequences in T-cells of the subject to generate a TCRβ clonotype profile of the subject; comparing the TCRβ clonotype profile of the subject to a database of target associated receptor sequences (TARSs) comprising unique TCRβ alleles identified as associated with exposure to the immunogenic antigen in a cohort of independent test subjects; generating a diagnostic classifier of the subject comprising the number of TARSs identified in the subject relative to the total number of unique TCRβ alleles in the subject; and determining that the subject has been exposed to the immunogenic antigen if the diagnostic classifier exceeds a predetermined threshold, wherein the predetermined threshold is determined by the prevalence of TARSs in the test cohort after exposure to the immunogenic antigen.

Also provided is a method for testing the efficacy of a vaccine. The method comprises: amplifying and sequencing TCRβ alleles in mRNA and/or genomic DNA of T-cells obtained from the subject after administration of the vaccine; comparing the TCRβ clonotype profile of the subject to a database of vaccine associated TCRβ sequences (VATSs) statistically associated with vaccination to generate a diagnostic classifier of the subject, wherein the diagnostic classifier comprises the number of VATSs identified in the subject relative to the total number of unique TCRβ alleles in the subject; and determining that the vaccine is effective in generating an immune response if the diagnostic classifier exceeds a threshold determined by the prevalence of VATSs in an independent test cohort after exposure to the vaccine.

Also provided is a method of identifying a viral infection in a subject. The method comprises: amplifying and sequencing TCRβ alleles in mRNA and/or genomic DNA of T-cells obtained from the subject; comparing the TCRβ sequences in the subject to one or more databases of virus-associated TCRβ sequences, wherein each database comprises TCRβ sequences statistically associated with one virus and each database is generated according to the methods described herein; and identifying the viral infection of the subject by determining the strength of the association of the TCRβ allele sequences identified in the subject to one or more of the databases.

A further method of identifying an immune response in a subject is provided, the method comprising: identifying in the subject the presence of a significant number of unique TCRβ clonotypes that match a database of TCRβ sequences previously associated with the immune response in an independent cohort.

In all of the methods provided herein, a TCRβ database is generated. Accordingly, a method of generating a TCRβ database is also provided, wherein the TCRβ database comprises TCRβ sequences statistically associated with an immune condition, exposure to a vaccine or immunogenic agent, and/or a pathogen, the method comprising: amplifying and sequencing TCRβ alleles in mRNA and/or genomic DNA of T-cells obtained from a cohort of subjects having the immune condition, or having been exposed to the vaccine, immunogenic agent and/or pathogen; and using a machine learning and/or neural network system to analyze the TCRβ allele sequences and statistically associate a subset of the TCRβ sequences to the immune condition, vaccine, immunogenic agent and/or pathogen.

Other objects and features will be in part apparent and in part pointed out hereinafter.

### BRIEF DESCRIPTION OF THE FIGURES

FIG. 1A illustrates the experimental workflow comprising using high throughput TCR sequencing to survey circulating TCR repertoires of mice before and after Orthopoxvirus infection.

FIG. 1B illustrates the experimental workflow comprising computationally identifying virus-associated TCR sequences in mice from FIG. 1A.

FIG. 1C shows the experimental workflow wherein virus-associated TCR sequences identified in FIG. 1B are used to diagnose de novo populations.

FIG. 1D shows the experimental workflow wherein virus-associated TCR sequences identified in FIG. 1B are used to track the virus-associated TCRs over time.

FIG. 2A is a flow diagram depicting the methodology of sample collection, vaccination and infection, DNA extraction and immunosequencing.

FIG. 2B shows scatter plots showing levels of pox specific antibodies from HLA-A2 humanized mice before or after ACAM2000 smallpox vaccination (left, n=29) or monkeypox virus (MPXV) infection (right; n=29).

FIG. 3A is a line graph displaying a representative assortment of vaccine-associated (black lines) or non-vaccine associated (grey lines) T cell receptor β (TCRO) clonotypes. Each line represents a unique TCRβ clonotype.

FIG. 3B shows the expansion of TCRβ sequences from splenocytes of vaccinated mice cultured with (black bars) or without (white bars) ACAM2000 either found in mice pre-vaccination (non-vaccine-associated) or identified in both 2 and 8 weeks post-vaccination TCRβ repertoires but absent pre-vaccination (post-vaccine-associated) (n=29 mice). Significance was calculated using chi-square test with Yates's correction (***p<0.0001).

FIG. 3C is a tabular representation of public TCRβ clonotypes enriched in vaccinated versus naive samples. The p value is calculated using one-tailed Fisher's exact test.

FIG. 4A is a scatterplot depicting the number of vaccine-associated TCRβ sequences (VATS) present in a sample against the total number of unique TCRβ clonotypes present in vaccinated (black dots) versus naive (gray dots) repertoires. The r value represents the Pearson correlation.

FIG. 4B is a bar graph displaying the distribution of % VATS in vaccinated (black bars) versus naive (white bars) samples.

FIG. 4C is a receiver operating characteristic (ROC) graph illustrating the accuracy of the diagnostic classifier at various discrimination thresholds. The graph plots the sensitivity (true positives) against the false discovery rate (FDR; false positives) as the discrimination threshold varies. The area under the ROC curve (AUROC) is a representation of the overall accuracy. The red dot represents the calculated discrimination threshold. The data table within the ROC graph displays the actual identity of the sample(s) (rows) versus how the sample was classified by the diagnostic assay (columns). Thirty-two of 32 naive samples and all 58 of 58 vaccinated samples (p=4.2×10-25) were correctly classified.

FIG. 4D is a bar graph displaying the distribution of % VATS in mice 16 weeks (dark gray bars) and 9 months (light gray bars) after vaccination compared with naive (white bars) and vaccinated (black bars) training data. The data table displays the actual identity of samples versus the sample's classification by the diagnostic assay. The assay correctly predicted 18 of 18 (100%) 16 weeks and 22 of 23 (96%) 9 month post-vaccination samples (p=6.5×10-20).

FIG. 5A is an ROC curve representing the overall accuracy of the diagnostic classifier to distinguish between naive samples and ACAM2000 vaccinated samples from the leave-one-out analyses (gray) compared to data from the full training set (black). Graphical representation of % VATS from mice pre- (naive, gray, n=32) or post- (vaccinated, black, n=29 mice) vaccination in the LOO analyses.

FIG. 5B is ROC curve comparing the overall accuracy of the diagnostic classifier from the full training set (black), LOO analysis (dark gray), and data from an independent cohort of mice pre- and post-ACAM2000 smallpox vaccination (light gray, n=20). Tabular results of the diagnostic classification of the independent cohort of ACAM2000 vaccinated mice. 18 of 20 (90%) naive samples were correctly classified, as were 19 of 20 (95%) samples from mice post-vaccination.

FIG. 5C is a bar graph showing a comparison of the % VATS in naïve (left) and vaccinated (right) samples from the LOO analyses (black) and independent cross-validation cohort of ACAM2000-vaccinated mice (gray).

FIG. 6A is a bar graph displaying the distribution of VATS of mice 2 and 8 weeks (dark gray bars; n=58), 16 weeks (light gray bars; n=29), and 9 months (shaded bars; 27 of 27) post-MPXV infection compared with the vaccinated (black bars) or naive (white bars) training data.

FIG. 6B is a ROC curve representing the overall accuracy of the diagnostic classifier to distinguish between naive samples and samples from mice infected with MPXV at the various time points post-infection. Overall, 55 of 58 (95%) of samples 2 and 8 weeks post-infection and 100% of samples 16 weeks (29 of 29) and 9 months (27 of 27) post-infection were correctly differentiated from naive samples.

FIG. 7A is a graphical representation of % MATS in mice 2-weeks and 8-weeks after infection with MPXV (black), vaccination with ACAM2000 smallpox vaccine (grey) or naïve mice (white).

FIG. 7B is an ROC curve representing the overall accuracy of the diagnostic classifier to distinguish between naïve samples and samples from mice infected with MPXV (black) or vaccinated with ACAM2000 (gray). In a leave-one-out (LOO) analysis, 31 of 32 (97%) naïve samples were correctly classified, as were 58 of 58 (100%) samples from mice 2- and 8-week post-infection. 56 of 58 (96.5%) samples 2- and 8-weeks post-vaccination were correctly differentiated from naïve samples.

FIG. 8A shows the frequency of VATS in mice vaccinated with the ACAM2000 smallpox vaccine over time. Each line represents the summed frequency of VATS in a single mouse from 2 weeks to 9 months post-vaccination. Dark dotted line represents the mean frequency of VATS in naive TCR repertoires SD (light dotted lines). Significance (p<0.0001) was determined using one-way ANOVA testing with Bonferroni's multiple comparison test.

FIG. 8B is a graphical representation of the expansion of VATS after in vitro culture with (black bar) or without (white bar) ACAM2000. Represented as the summed frequency (percentage of all TCRβ sequences) of VATS. Significance was calculated using chi-square test with Yates's correction.

FIG. 8C shows representative flow plots displaying irrelevant tetramer (top) and nine pooled HLA-A2 tetramers loaded with vaccinia-specific peptides (bottom) binding to CD8+ T cells.

FIG. 8D is a bar graph displaying the proportion of tetramer− or tetramer+ sequences that were included in the VATS library. The p value is calculated using a two-tailed Fisher's exact test.

FIG. 8E is a graph showing the summed frequency of tetramer+ VATS in ACAM2000-vaccinated mice over time. Each line represents the frequency of the tetramer+ VATS in an individual mouse from prior to vaccination through 9 months post-vaccination. Significance (p<0.0001) was determined using one-way ANOVA testing with Bonferroni's multiple-comparison test.

FIG. 9 is an illustration of iCAT using TCR repertoires from mouse blood samples of pre (negative) and post (positive) exposure of smallpox virus infection.

FIG. 10 is a flowchart of a deep neural network (DNN) model and prediction for viral infection diagnosis.

FIG. 11A shows a flow chart depicting the purification of genomic DNA from blood samples and the production of TCR repertoires after TCR-specific amplification and sequencing.

FIG. 11B is a visual representation of data preprocessing and DNN architecture.

FIG. 11C shows model building and prediction results of the human dataset.

### DETAILED DESCRIPTION OF THE INVENTION

The methods provided herein are directed to examining the T-cell receptor (TCR) repertoire of the subject. During T cell development, each TCR chain is generated through quasi-random genetic recombination from the germline loci of the variable (V), diversity (D), and joining (J) gene segments. T-cells express antigen specific TCRs which are expressed from a highly polymorphic TCR gene locus comprising V, D and J gene segments. On average, approximately 10⁷ unique TCRβ chains can be identified from the approximately 10¹² circulating T cells present in a healthy human adult. The ability to readily identify identical TCR sequences among multiple individuals (public TCRs) is challenging because an individual has the potential to generate approximately 10¹⁸ unique TCR recombinants. Moreover, there is no guarantee that two individuals will express the same TCR to the same antigen. Further, identifying TCR sequences that correlate with an infection can be more difficult the more time passes from the infection as clonally expanded T-cells that were upregulated during the initial immune response are depleted, leaving only a small population of memory T-cells. The present invention addresses each of these issues.

As noted above, a method is provided herein for determining whether a subject has been exposed to an immunogenic antigen. As used herein, the term “immunogenic antigen” comprises any antigen that elicits a robust immune response. In general, the robust immune response comprises humoral and cell-mediated immunity (e.g., upregulation of antigen-specific B- and T-cells in the subject, respectively).

### Method for Determining Exposure Status of a Subject

Accordingly, in various embodiments, the methods described herein comprise amplifying and sequencing TCRβ alleles in mRNA and/or genomic DNA obtained from T-cells isolated from the subject. TCRβ alleles are well characterized in the art as are methods of amplifying and expanding. For example, the multiplex method of isolating TCRβ genes may be carried out according to previously published methods (e.g., using multiplexed primers targeting all V and J gene segments as described by Carlson et al., 2013, 2013, “Using synthetic templates to design an unbiased multiplex PCR assay. Nat. Comm. 4, 2680 and incorporated herein by reference in its entirety). The genetic diversity of the population (e.g., humans) may require increased sequencing depth. Accordingly, the sequencing may further comprise an ultra-deep sequencing protocol to achieve read depths up of at least about 2 million, at least about 3 million, or at least about 5 million reads. For example, the sequencing can be performed at a depth of from about 2 million to about 100 million reads, from about 2 million to about 10 million reads, from about 2 million to about 5 million reads, from about 4 million to about 100 million reads, from about 4 million to about 10 million reads, from about 4 million to about 6 million reads, or from about 4 million to about 5 million reads.

Once the TCRβ alleles are amplified and sequenced, the method further comprises identifying unique TCRβ alleles in the samples to generate a TCRβ clonotype profile. As used herein, the word ‘unique” means a unique sequence among the total number of TCRβ sequences identified. The word “unique” does not imply that the identified sequences have multiple copies in the original sample.

In various embodiments, the TCRβ clonotype profile (e.g., unique TCRβ allele sequences identified in the sample) is compared to a database of target associated receptor sequences (TARSs) comprising unique TCRβ allele sequences statistically associated with the immunogenic antigen in an independent cohort of test subjects to generate a diagnostic classifier of the sample. The diagnostic classifier comprises the number of TARSs identified in the subject relative to the total number of unique TCRβ alleles in the subject.

In further embodiments, the method comprises determining that the subject has been exposed to the immunogenic antigen if the diagnostic classifier exceeds a predetermined threshold for the diagnostic classifier, wherein the predetermined threshold is determined by the prevalence of TARSs in the test cohort after exposure to the immunogenic antigen.

The method described herein therefore comprises two steps of (a) preparing a database of TCRβ sequences associated with the immunogenic antigen and (b) comparing the TCRβ sequences of the subject to be evaluated with that database. Each of these steps are described in more detail below.

Preparing a Database of TCRβ Sequences Associated with an Immunogenic Antigen

In various embodiments, generating the database of “target associated receptor sequences” TARSs comprises analyzing the shared immune response of an independent cohort of test subjects following an exposure to the antigen. Accordingly, the method can comprise amplifying and sequencing TCRβ alleles in mRNA and/or genomic DNA obtained from T cells of the test subjects, wherein the T cells are isolated before and after exposure to the immunogenic antigen; identifying unique TCRβ allele sequences in the cohort of test subjects; performing a Fisher exact test on each unique TCRβ sequence to generate a statistical association (i.e., a p-value) between the TCRβ sequence and the exposure status of the subject at the time the T-cells were obtained (that is, whether the T-cell sample as collected “before” or “after” exposure); generating a database of TARSs comprising unique TCRβ sequences having a p-value that exceeds a p-value threshold.

In various embodiments, the p-value threshold is determined empirically for the cohort of test subjects used. Specifically, the p-value threshold is the p-value that generates a TARSs database having the maximum coverage ratio. As used herein, the term “coverage ratio” is defined as the ratio of “Cp” to “Cn”, wherein “Cp” and “Cn” are, respectively, the proportion of exposed (Cp) or naïve (Cn) samples having at least one TCRβ sequence included in the TARSs database relative to the total number of exposed samples (when calculating “Cp”) or naïve samples (when calculating “Cn”). In other words, a coverage ratio can be calculated using the following equation, where Cv represented Cp as described above, Cn is as described above, and “xᵢ” and yᵢ represent the total number of exposed samples or naïve samples, respectfully, that a single TCRβ is identified in and nᵥ and nₙ represent the total number of exposed samples or naïve samples, respectfully:

$${C}_{v} = \frac{{\sum }_{i = 1}^{I} {x}_{i}}{{n}_{v}} {C}_{n} = \frac{{\sum }_{i = 1}^{I} {y}_{i}}{{n}_{n}}$$

Accordingly, the p-value threshold can be determined by sorting TCRβ sequences into “exposed”-associated or “naïve”-associated groups using a range of p value thresholds (although p values tested should not exceed 0.20). The coverage ratio can be calculated for each p value and the p value that yields a maximum (e.g., highest) coverage ratio can then be selected as the p-value threshold to define the final TCRβ database associated with the immunogenic antigen. Importantly, the final TCRβ database is generally considered to be static and is not altered when an unknown subject must be classified. Accordingly, in some embodiments, the TCRβ database is not regenerated every time an unknown subject is classified.

Preferably, generating the TCRβ database associated with the immunogenic antigen comprises using a machine learning or neural network platform that efficiently sorts TCRβ sequences into “exposed” or “naïve” classes. Although the Fisher exact test is provided as an exemplary statistical test to classify the sequences, other statistical tests and methods may be used. Preferably, generating the TCRβ database comprises using a neural network or machine learning interface that trains on data gathered from naïve or exposed samples and determines relationships between the TCRβ allele sequences and their association with exposure to the antigen.

In various embodiments, the method of generating the TARSs database further comprises validating the database by identifying one or more splenocytes present in the test subjects of the cohort after exposure to the immunogenic antigen that express one or more of the TARSs in the database. In various embodiments, the splenocytes may be identified using an in vitro clonal expansion experiment where splenocytes are exposed to the immunogenic antigen in vitro, clonally expand and are analyzed to determine the sequences of their expressed TCRβ chains. In other embodiments, splenocytes may be analyzed in a flow cytometry procedure where MHC-peptide tetramers are used to bind to and label T-cell receptors on the splenocytes. In this embodiment, the MHC-peptide tetramers are the extracellular binding domain of the major histocompatibility complex (MHC) associated with an antigen peptide. Preferably, the antigen peptide is associated with (or mirrors) the immunogenic antigen used to generate the TARSs database. In various embodiments, the MHC antigen peptide can comprise any one of SEQ ID NO: 675-683. The MHC protein can comprise a human leukocyte antigen peptide (e.g., HLA-A2). Splenocytes that are isolated using this method can be further analyzed to determine their TCRβ sequences to determine whether they match the TCRβ sequences on the database.

### Classifying an Unknown Sample

As described above, the methods provided comprise classifying a subject of unknown status as either exposed or naïve depending on whether its diagnostic classifier exceeds a predetermined threshold. In various embodiments, the comparison of the diagnostic classifier with the predetermined threshold further comprises applying a probability distribution function that compares the diagnostic classifier of the subject to a distribution of TARSs prevalence in the test subject cohort after exposure to the immunogenic antigen. As used herein, “prevalence” refers to the ratio of unique TARSs identified in each sample relative to the total number of unique TCRβ sequences in each sample.

Accordingly, the methods described herein enable one to evaluate an unknown subject against a predetermined database of TCRβ sequences associated with exposure to the immunogenic antigen. Importantly, since this database is evaluated independently of the test subject, the immune profile of the subject can be re-evaluated through time. Accordingly, the methods described herein can further comprise dynamically tracking an immune response to the subject over time, the method comprising generating a plurality of diagnostic classifier scores using T-cell samples obtained from the subject at different time points and comparing the diagnostic classifiers to a TARSs database associated with the immune response. Further, generating the diagnostic classifiers of the subject does not alter the TARSs database.

In various embodiments, the methods comprise analyzing a sample of T-cells obtained from the subject up to 9 months after a potential exposure event to the immunogenic antigen. For example, in various embodiments, the sample of T-cells may be obtained around 2 weeks, around 4 weeks, around 6 weeks, around 12 weeks, around 24 weeks, and/or around 36 weeks after the potential exposure event to the immunogenic antigen. In various embodiments, the T cells can comprise CD8+ T cells.

### TCRβ Alleles

As noted above TCRβ alleles are unique for each T-cell and are generated via thymic recombination of various V, D and J regions of the TCR gene. Accordingly, the TCRβ allele can comprise the associated V region and J region of the TCR gene and the corresponding CDR3 sequence that spans the two. Further, as would be understood by one of skill in the art, once a genomic allele is determined the corresponding amino acid sequence encoded by that allele is easy to obtain. Consequently, as used herein, the word “allele” refers to the gene as provided in DNA or transcribed to mRNA, as well as the gene expressed into protein (amino acid sequence). As used herein, the TCRβ sequences are represented using nomenclature established by the international ImMunoGeneTics (IMGT) system (www.imgt.org). In this system, the variable (v) and joining (j) genes are named and the hypervariable region that spans them (CDR3) is provided as an amino acid sequence. For example, a TCRβ sequence can be represented as: “TCRBV03-01 CASSLGFYEQYF TCRBJ02-07”. In this nomenclature, “TCRBV03-01” and TCRBJ02-07 represent the IMGT classified name for the “v” and J regions, respectfully, and can be identified from public databases (e.g., imgt.org). The sequence CASSLGFYEQYF is the hypervariable CDR3 region and is assigned SEQ ID NO: 121 herein. Accordingly, once provided with an allele name (V-CDR3-J) one can identify the underlying sequence easily using a database such as found on www.imgt.org.

For example, given the V-CDR3-J name, one can obtain the corresponding TRBV and TCRBJ segments (as amino acid sequences) and align the end of the TRBV sequence to the beginning of the CDR3 sequence and the beginning of the TRBJ sequence to the end of the CDR3 sequence to find overlapping amino acid sequences and then combine into a single sequence.

In various embodiments, the TCRβ allele comprises a CDR3 variable region in a recombined TCRβ allele. The CDR3 variable region can comprise an amino acid sequence comprising any one of SEQ ID NOs: 1-674. In further embodiments, the TCRβ allele comprises the V region, the CDR3 variable region and the J region of a recombined TCRβ allele.

### Immunogenic Antigens

The methods described herein may be used to determine whether a subject has been exposed to an immunogenic antigen. In various embodiments, the immunogenic antigen can comprise a pathogen, an allergen, a vaccine, a virus or any immunogenic component or fragment thereof. In some embodiments, the methods comprise identifying an immune response in the subject, provided the immune response is mediated by T-cell upregulation.

In various embodiments, the immunogenic antigen comprises a virus or a vaccine. For example, the immunogenic antigen can comprise an Orthopoxvirus (e.g., smallpox or monkey pox), a coronavirus (e.g., SARS-COV, SARS-COV-2, or MERS), an influenza virus (e.g., Influenza A or Influenza B). As another example, the immunogenic antigen can comprise a vaccine to any of these viruses. So, for example, the immunogenic antigen can comprise an Orthopoxvirus vaccine (e.g., the smallpox vaccine or another Orthopoxvirus vaccine), a coronavirus vaccine (e.g., a SARS COV-2 vaccine) or an influenza vaccine.

In various embodiments, when the immunogenic antigen comprises an Orthopoxvirus (e.g., monkey pox), the TARSs database comprising TCRβ allele sequences associated with the infection (e.g., with monkey pox) can comprise any one of SEQ ID NOs: 1-120. As noted above, the TCRβ allele sequences are annotated to indicate the “V” gene, the “J” gene and the CDR3 amino acid sequence that comprises the final recombined allele. Each of the CDR3 sequences is assigned a SEQ ID NO. For ease of reference, SEQ ID NOs: 1-120 are indicated in Table 1 below. The TARSs associated with monkey pox infection and provided in Table 1 comprise murine TCRβ alleles.

TABLE 1
TCRβ alleles associated with monkey pox infection

| | CDR3 |
|---|---|
| | SEQ ID |
| V-CDR3-J (mus musculus) | NO: |
| TCRBV13-01 CASSDPGLGDYEQYF TCRBJ02-07 | 1 |
| TCRBV14-01 CASSSTGYNNQAPLF TCRBJ01-05 | 2 |
| TCRBV01-01 CTCSAEGGANTEVFF TCRBJ01-01 | 3 |
| TCRBV04-01 CASSLGLGNYAEQFF TCRBJ02-01 | 4 |
| TCRBV04-01 CASSLTGGNTEVFF TCRBJ01-01 | 5 |
| TCRBV05-01 CASSPRDREDTQYF TCRBJ02-05 | 6 |
| TCRBV02-01 CASSPDRDEQYF TCRBJ02-07 | 7 |
| TCRBV02-01 CASSQDGANTGQLYF TCRBJ02-02 | 8 |
| TCRBV03-01 CASSLEQNQAPLF TCRBJ01-05 | 9 |
| TCRBV03-01 CASSPTGNTEVFF TCRBJ01-01 | 10 |
| TCRBV04-01 CASSRSYNSPLYF TCRBJ01-06 | 11 |
| TCRBV05-01 CASSPGTEVFF TCRBJ01-01 | 12 |
| TCRBV05-01 CASSQDITEVFF TCRBJ01-01 | 13 |
| TCRBV05-01 CASSQDWVNYAEQFF TCRBJ02-01 | 14 |
| TCRBV12-01 CASSLGETLYF TCRBJ02-03 | 15 |
| TCRBV13-01 CASSDAGEEQYF TCRBJ02-07 | 16 |
| TCRBV13-02 CASGAGGEDTQYF TCRBJ02-05 | 17 |
| TCRBV13-02 CASGDTGAGNTLYF TCRBJ01-03 | 18 |
| TCRBV13-02 CASGEGLGKDTQYF TCRBJ02-05 | 19 |
| TCRBV13-02 CASGPTFNQDTQYF TCRBJ02-05 | 20 |
| TCRBV14-01 CASSFTGGNNQAPLF TCRBJ01-05 | 21 |
| TCRBV16-01 CASSLAGNERLFF TCRBJ01-04 | 22 |
| TCRBV19-01 CASSIGTGGNTGQLYF TCRBJ02-02 | 23 |
| TCRBV26-01 CASSLRGTGNTLYF TCRBJ01-03 | 24 |
| TCRBV26-01 CASSLTGGSNERLFF TCRBJ01-04 | 25 |
| TCRBV29-01 CASSLRDIYEQYF TCRBJ02-07 | 26 |
| TCRBV31-01 CAWSLDRYNSPLYF TCRBJ01-06 | 27 |
| TCRBV31-01 CAWSLPNSGNTLYF TCRBJ01-03 | 28 |
| TCRBV01-01 CTCSAAGTGVGNTLYF TCRBJ01-03 | 29 |
| TCRBV01-01 CTCSADRGSYEQYF TCRBJ02-07 | 30 |
| TCRBV01-01 CTCSAEDWGNYAEQFF TCRBJ02-01 | 31 |
| TCRBV01-01 CTCSAGGSNTEVFF TCRBJ01-01 | 32 |
| TCRBV01-01 CTCSAGRNSPLYF TCRBJ01-06 | 33 |
| TCRBV01-01 CTCSARTGGAGEQYF TCRBJ02-07 | 34 |
| TCRBV02-01 CASSQDGRGEQYF TCRBJ02-07 | 35 |
| TCRBV02-01 CASSQDRTGNTEVFF TCRBJ01-01 | 36 |
| TCRBV02-01 CASSQGGGTEVFF TCRBJ01-01 | 37 |
| TCRBV03-01 CASSFQANTEVFF TCRBJ01-01 | 38 |
| TCRBV03-01 CASSLARGYEQYF TCRBJ02-07 | 39 |
| TCRBV03-01 CASSLDSSNTEVFF TCRBJ01-01 | 40 |
| TCRBV03-01 CASSLGQGGGNTLYF TCRBJ01-03 | 41 |
| TCRBV03-01 CASSLKGQDTQYF TCRBJ02-05 | 42 |
| TCRBV03-01 CASSLSANTEVFF TCRBJ01-01 | 43 |
| TCRBV03-01 CASSQTGGAREQYF TCRBJ02-07 | 44 |
| TCRBV03-01 CASSYRNTEVFF TCRBJ01-01 | 45 |
| TCRBV04-01 CASRTISNERLFF TCRBJ01-04 | 46 |
| TCRBV04-01 CASSFDRGEVFF TCRBJ01-01 | 47 |
| TCRBV04-01 CASSPDWGGNTGQLYF TCRBJ02-02 | 48 |
| TCRBV04-01 CASSPLGVNQDTQYF TCRBJ02-05 | 49 |
| TCRBV04-01 CASSPTAYEQYF TCRBJ02-07 | 50 |
| TCRBV05-01 CASSQEGQGGDTQYF TCRBJ02-05 | 51 |
| TCRBV05-01 CASSQGDSSAETLYF TCRBJ02-03 | 52 |
| TCRBV05-01 CASSQGLSNERLFF TCRBJ01-04 | 53 |
| TCRBV05-01 CASSQLGGNTGQLYF TCRBJ02-02 | 54 |
| TCRBV12-01 CASSGQSNERLFF TCRBJ01-04 | 55 |
| TCRBV12-01 CASSLAGGGQNTLYF TCRBJ02-04 | 56 |
| TCRBV12-01 CASSLPTNSDYTF TCRBJ01-02 | 57 |
| TCRBV12-01 CASSLTGDYEQYF TCRBJ02-07 | 58 |
| TCRBV12-01 CASSLTNQDTQYF TCRBJ02-05 | 59 |
| TCRBV12-01 CASSWDWGSQNTLYF TCRBJ02-04 | 60 |
| TCRBV12-02 CASSLEGGSSYEQYF TCRBJ02-07 | 61 |
| TCRBV12-02 CASSLGLGVYAEQFF TCRBJ02-01 | 62 |
| TCRBV12-02 CASSLRGNTLYF TCRBJ01-03 | 63 |
| TCRBV12-02 CASSPDSGNTLYF TCRBJ01-03 | 64 |
| TCRBV12-02 CASSPGQGSDYTF TCRBJ01-02 | 65 |
| TCRBV13-01 CASRLGANTGQLYF TCRBJ02-02 | 66 |
| TCRBV13-01 CASSDAGLGFYEQYF TCRBJ02-07 | 67 |
| TCRBV13-01 CASSDAYSGNTLYF TCRBJ01-03 | 68 |
| TCRBV13-01 CASSDPGLGFYEQYF TCRBJ02-07 | 69 |
| TCRBV13-01 CASSDSANTGQLYF TCRBJ02-02 | 70 |
| TCRBV13-01 CASSETGNYAEQFF TCRBJ02-01 | 71 |
| TCRBV13-02 CASGAGAGNTLYF TCRBJ01-03 | 72 |
| TCRBV13-02 CASGDAGEQDTQYF TCRBJ02-05 | 73 |
| TCRBV13-02 CASGDARGENTLYF TCRBJ02-04 | 74 |
| TCRBV13-02 CASGDFNSPLYF TCRBJ01-06 | 75 |
| TCRBV13-02 CASGDRFSYEQYF TCRBJ02-07 | 76 |
| TCRBV13-02 CASGEAGDYAEQFF TCRBJ02-01 | 77 |
| TCRBV13-02 CASGPGQSNTEVFF TCRBJ01-01 | 78 |
| TCRBV13-03 CASSDAGSNERLFF TCRBJ01-04 | 79 |
| TCRBV13-03 CASSDATGGYEQYF TCRBJ02-07 | 80 |
| TCRBV13-03 CASSGTGVSYEQYF TCRBJ02-07 | 81 |
| TCRBV14-01 CASSFTGQNNQAPLF TCRBJ01-05 | 82 |
| TCRBV14-01 CASSFTGRNNQAPLF TCRBJ01-05 | 83 |
| TCRBV15-01 CASSLDKNTGQLYF TCRBJ02-02 | 84 |
| TCRBV15-01 CASSLGVYEQYF TCRBJ02-07 | 85 |
| TCRBV15-01 CASSLRGSGNTLYF TCRBJ01-03 | 86 |
| TCRBV15-01 CASSPGQYAEQFF TCRBJ02-01 | 87 |
| TCRBV16-01 CASSWGGNQDTQYF TCRBJ02-05 | 88 |
| TCRBV17-01 CASSRRQYEQYF TCRBJ02-07 | 89 |
| TCRBV19-01 CASSIRDWGGAEQFF TCRBJ02-01 | 90 |
| TCRBV19-01 CASSLTGNNQAPLF TCRBJ01-05 | 91 |
| TCRBV19-01 CASSMTGGSQNTLYF TCRBJ02-04 | 92 |
| TCRBV19-01 CASSRDKQDTQYF TCRBJ02-05 | 93 |
| TCRBV20-01 CGARDRGKNTLYF TCRBJ02-04 | 94 |
| TCRBV20-01 CGARVGSAETLYF TCRBJ02-03 | 95 |
| TCRBV23-01 CSSSQTNTGQLYF TCRBJ02-02 | 96 |
| TCRBV26-01 CASSLQKNTEVFF TCRBJ01-01 | 97 |
| TCRBV26-01 CASSLSRANSDYTF TCRBJ01-02 | 98 |
| TCRBV26-01 CASSLYRAGNTLYF TCRBJ01-03 | 99 |
| TCRBV26-01 CASSQDSYNSPLYF TCRBJ01-06 | 100 |
| TCRBV26-01 CASSRGVSGNTLYF TCRBJ01-03 | 101 |
| TCRBV29-01 CASSFGQGNTEVFF TCRBJ01-01 | 102 |
| TCRBV29-01 CASSFGSNERLFF TCRBJ01-04 | 103 |
| TCRBV29-01 CASSLGDSNERLFF TCRBJ01-04 | 104 |
| TCRBV29-01 CASSLGTGYAEQFF TCRBJ02-01 | 105 |
| TCRBV29-01 CASSLRDRNTGQLYF TCRBJ02-02 | 106 |
| TCRBV29-01 CASSRQGANSDYTF TCRBJ01-02 | 107 |
| TCRBV29-01 CASSSGTGSNERLFF TCRBJ01-04 | 108 |
| TCRBV29-01 CASSTGTEVFF TCRBJ01-01 | 109 |
| TCRBV31-01 CAWKGQSNSDYTF TCRBJ01-02 | 110 |
| TCRBV31-01 CAWSLEGRDTQYF TCRBJ02-05 | 111 |
| TCRBV31-01 CAWSPRDTQYF TCRBJ02-05 | 112 |
| TCRBV31-01 CAWSQGGNSDYTF TCRBJ01-02 | 113 |
| TCRBV12-01 CASSPGISNERLFF TCRBJ01-04 | 114 |
| TCRBV02-01 CASSQGGNSDYTF TCRBJ01-02 | 115 |
| TCRBV05-01 CASSQEGGVNQDTQYF TCRBJ02-05 | 116 |
| TCRBV31-01 CAWSLGGVYEQYF TCRBJ02-07 | 117 |
| TCRBV31-01 CAWSLQANTEVFF TCRBJ01-01 | 118 |
| TCRBV04-01 CASSRDSQNTLYF TCRBJ02-04 | 119 |
| TCRBV15-01 CASSLEGGNTEVFF TCRBJ01-01 | 120 |

In various embodiments, the immunogenic antigen comprises a vaccine (e.g., a smallpox vaccine). For example, the immunogenic antigen can comprise the ACAM2000 smallpox vaccine. In various embodiments, when a TCRβ allele on the TARSs database that is associated with the smallpox vaccine can comprise any one of SEQ ID NOs: 121-435. For ease of reference SEQ ID NOs: 121-435 are provided in Table 2 below. As above, individual clonotypes are identified using IMGT standard nomenclature (V-CDR3-J). The international ImMunoGenTics database is available (www.imgt.org) and can be used to generate the raw sequences provided below. The TARSs associated with smallpox vaccination and provided in Table 2 comprise murine TCRβ alleles.

TABLE 2
TCRβ alleles associated with smallpox vaccination.

| | CDR3 |
|---|---|
| | SEQ ID |
| V-CDR3-J (mus musculus) | NO: |
| TCRBV03-01 CASSLGFYEQYF TCRBJ02-07 | 121 |
| TCRBV19-01 CASSRDKQDTQYF TCRBJ02-05 | 122 |
| TCRBV14-01 CASSSTGYNNQAPLF TCRBJ01-05 | 123 |
| TCRBV01-01 CTCSAEGVSNERLFF TCRBJ01-04 | 124 |
| TCRBV14-01 CASSFTGQNNQAPLF TCRBJ01-05 | 125 |
| TCRBV13-01 CASSRQGGDERLFF TCRBJ01-04 | 126 |
| TCRBV29-01 CASGNTEVFF TCRBJ01-01 | 127 |
| TCRBV13-03 CASSDAGAEQFF TCRBJ02-01 | 128 |
| TCRBV14-01 CASSFTGRNNQAPLF TCRBJ01-05 | 129 |
| TCRBV19-01 CASSRDRYAEQFF TCRBJ02-01 | 130 |
| TCRBV01-01 CTCSADLGTSAETLYF TCRBJ02-03 | 131 |
| TCRBV12-02 CASSPTTSAETLYF TCRBJ02-03 | 132 |
| TCRBV04-01 CASSHRDGQDTQYF TCRBJ02-05 | 133 |
| TCRBV13-02 CASGEGLGEQYF TCRBJ02-07 | 134 |
| TCRBV05-01 CASSQDRQGYEQYF TCRBJ02-07 | 135 |
| TCRBV03-01 CASSSDRHQDTQYF TCRBJ02-05 | 136 |
| TCRBV05-01 CASSQDLGPYEQYF TCRBJ02-07 | 137 |
| TCRBV19-01 CASSIRAEQYF TCRBJ02-07 | 138 |
| TCRBV12-02 CASSLTGGSSYEQYF TCRBJ02-07 | 139 |
| TCRBV01-01 CTCSAAGTGVGNTLYF TCRBJ01-03 | 140 |
| TCRBV04-01 CASSLTAYEQYF TCRBJ02-07 | 141 |
| TCRBV05-01 CASSQEGLGGREQYF TCRBJ02-07 | 142 |
| TCRBV13-03 CASSDPGGNERLFF TCRBJ01-04 | 143 |
| TCRBV05-01 CASSQEGINQDTQYF TCRBJ02-05 | 144 |
| TCRBV12-01 CASSLGTVSYNSPLYF TCRBJ01-06 | 145 |
| TCRBV05-01 CASSQETGNTEVFF TCRBJ01-01 | 146 |
| TCRBV31-01 CAWSLAGDNQAPLF TCRBJ01-05 | 147 |
| TCRBV05-01 CASSQEGTGTETLYF TCRBJ02-03 | 148 |
| TCRBV14-01 CASSSTGRNNQAPLF TCRBJ01-05 | 149 |
| TCRBV13-02 CASGDWGGATGQLYF TCRBJ02-02 | 150 |
| TCRBV13-02 CASGDAAGGTGQLYF TCRBJ02-02 | 151 |
| TCRBV19-01 CASSPTTYEQYF TCRBJ02-07 | 152 |
| TCRBV03-01 CASSLSGGYEQYF TCRBJ02-07 | 153 |
| TCRBV13-03 CASSPDSYEQYF TCRBJ02-07 | 154 |
| TCRBV05-01 CASSPGTNNQAPLF TCRBJ01-05 | 155 |
| TCRBV13-03 CASSPQGAGNTLYF TCRBJ01-03 | 156 |
| TCRBV04-01 CASSWTGSGNTLYF TCRBJ01-03 | 157 |
| TCRBV13-01 CASRLRDWGYEQYF TCRBJ02-07 | 158 |
| TCRBV02-01 CASSQDPGGGYEQYF TCRBJ02-07 | 159 |
| TCRBV19-01 CASSTGGVYEQYF TCRBJ02-07 | 160 |
| TCRBV29-01 CASSTSNSDYTF TCRBJ01-02 | 161 |
| TCRBV01-01 CTCSARDTYEQYF TCRBJ02-07 | 162 |
| TCRBV13-02 CASGGTGVYEQYF TCRBJ02-07 | 163 |
| TCRBV13-02 CASGTGGSYEQYF TCRBJ02-07 | 164 |
| TCRBV13-01 CASSDAIYEQYF TCRBJ02-07 | 165 |
| TCRBV03-01 CASSLAPDSGNTLYF TCRBJ01-03 | 166 |
| TCRBV04-01 CASSLRDGQDTQYF TCRBJ02-05 | 167 |
| TCRBV03-01 CASSSGDSDYTF TCRBJ01-02 | 168 |
| TCRBV01-01 CTCSARLGGYAEQFF TCRBJ02-01 | 169 |
| TCRBV12-01 CASSPPGQLYF TCRBJ02-02 | 170 |
| TCRBV01-01 CTCSAGGGAGEQYF TCRBJ02-07 | 171 |
| TCRBV13-01 CASRRQGNSDYTF TCRBJ01-02 | 172 |
| TCRBV13-01 CASSDGTEQYF TCRBJ02-07 | 173 |
| TCRBV13-03 CASSDQGSNERLFF TCRBJ01-04 | 174 |
| TCRBV16-01 CASSPTGGGNTLYF TCRBJ01-03 | 175 |
| TCRBV19-01 CASSRDNNYAEQFF TCRBJ02-01 | 176 |
| TCRBV31-01 CAWSRNSDYTF TCRBJ01-02 | 177 |
| TCRBV29-01 CASSFQQDTQYF TCRBJ02-05 | 178 |
| TCRBV15-01 CASSGDNAETLYF TCRBJ02-03 | 179 |
| TCRBV26-01 CASSLGLNQDTQYF TCRBJ02-05 | 180 |
| TCRBV13-02 CASGPGRISNERLFF TCRBJ01-04 | 181 |
| TCRBV13-03 CASSGTVNYAEQFF TCRBJ02-01 | 182 |
| TCRBV03-01 CASSLNSNSDYTF TCRBJ01-02 | 183 |
| TCRBV03-01 CASSPDSSAETLYF TCRBJ02-03 | 184 |
| TCRBV26-01 CASSPGQTEVFF TCRBJ01-01 | 185 |
| TCRBV29-01 CASSPTGSGNTLYF TCRBJ01-03 | 186 |
| TCRBV02-01 CASSQDGGGTGQLYF TCRBJ02-02 | 187 |
| TCRBV05-01 CASSQGYQDTQYF TCRBJ02-05 | 188 |
| TCRBV16-01 CASSFKDTQYF TCRBJ02-05 | 189 |
| TCRBV19-01 CASSIAGTGNERLFF TCRBJ01-04 | 190 |
| TCRBV12-01 CASSPDRGQNTLYF TCRBJ02-04 | 191 |
| TCRBV03-01 CASSWTGQDTQYF TCRBJ02-05 | 192 |
| TCRBV04-01 CASSYREDTQYF TCRBJ02-05 | 193 |
| TCRBV13-03 CASTGQANTEVFF TCRBJ01-01 | 194 |
| TCRBV01-01 CTCSADINQDTQYF TCRBJ02-05 | 195 |
| TCRBV13-02 CASGETGGNTEVFF TCRBJ01-01 | 196 |
| TCRBV13-02 CASGPGQSNTEVFF TCRBJ01-01 | 197 |
| TCRBV13-01 CASSGDNSAETLYF TCRBJ02-03 | 198 |
| TCRBV12-02 CASSLEAGGAETLYF TCRBJ02-03 | 199 |
| TCRBV12-01 CASSLQNTLYF TCRBJ02-04 | 200 |
| TCRBV26-01 CASSLRGEVFF TCRBJ01-01 | 201 |
| TCRBV03-01 CASSPGQGDTEVFF TCRBJ01-01 | 202 |
| TCRBV01-01 CTCSAGTGHTEVFF TCRBJ01-01 | 203 |
| TCRBV03-01 CASSPRTGGSAETLYF TCRBJ02-03 | 204 |
| TCRBV16-01 CASSLGTGVNQAPLF TCRBJ01-05 | 205 |
| TCRBV01-01 CTCSAGTKDTQYF TCRBJ02-05 | 206 |
| TCRBV04-01 CASSPTSYEQYF TCRBJ02-07 | 207 |
| TCRBV03-01 CASSLVGASAETLYF TCRBJ02-03 | 208 |
| TCRBV20-01 CGAREGEDTQYF TCRBJ02-05 | 209 |
| TCRBV02-01 CASSQDRDKYEQYF TCRBJ02-07 | 210 |
| TCRBV15-01 CASSRQGGDERLFF TCRBJ01-04 | 211 |
| TCRBV16-01 CASSLGGPYEQYF TCRBJ02-07 | 212 |
| TCRBV13-03 CASRNTGQLYF TCRBJ02-02 | 213 |
| TCRBV16-01 CASSRQGNYAEQFF TCRBJ02-01 | 214 |
| TCRBV29-01 CASSLGGANTGQLYF TCRBJ02-02 | 215 |
| TCRBV13-02 CASGDAGGRNTLYF TCRBJ02-04 | 216 |
| TCRBV13-02 CASGGGLQDTQYF TCRBJ02-05 | 217 |
| TCRBV03-01 CASSFDWGQDTQYF TCRBJ02-05 | 218 |
| TCRBV03-01 CASSLGLGVNQDTQYF TCRBJ02-05 | 219 |
| TCRBV12-02 CASSLGQSQNTLYF TCRBJ02-04 | 220 |
| TCRBV29-01 CASSLSGNQDTQYF TCRBJ02-05 | 221 |
| TCRBV03-01 CASSSGLQDTQYF TCRBJ02-05 | 222 |
| TCRBV31-01 CAWSPDRANTEVFF TCRBJ01-01 | 223 |
| TCRBV15-01 CASSLAGGNTEVFF TCRBJ01-01 | 224 |
| TCRBV16-01 CASSPGLGEDTQYF TCRBJ02-05 | 225 |
| TCRBV05-01 CASSQDGGASQNTLYF TCRBJ02-04 | 226 |
| TCRBV31-01 CAWSLDQDTQYF TCRBJ02-05 | 227 |
| TCRBV13-01 CASSEGSQDTQYF TCRBJ02-05 | 228 |
| TCRBV19-01 CASSSGTANTEVFF TCRBJ01-01 | 229 |
| TCRBV13-02 CASGDVGQGNERLFF TCRBJ01-04 | 230 |
| TCRBV29-01 CASSLPGTNERLFF TCRBJ01-04 | 231 |
| TCRBV26-01 CASSLSGNTGQLYF TCRBJ02-02 | 232 |
| TCRBV01-01 CTCSAGQNNQAPLF TCRBJ01-05 | 233 |
| TCRBV16-01 CASSLGGAREQYF TCRBJ02-07 | 234 |
| TCRBV13-03 CASSDLGGQDTQYF TCRBJ02-05 | 235 |
| TCRBV02-01 CASSQESQNTLYF TCRBJ02-04 | 236 |
| TCRBV13-01 CASSGTGGYAEQFF TCRBJ02-01 | 237 |
| TCRBV02-01 CASSQDNSQNTLYF TCRBJ02-04 | 238 |
| TCRBV12-01 CASSLGGAGNTLYF TCRBJ01-03 | 239 |
| TCRBV02-01 CASSQEGWGNQDTQYF TCRBJ02-05 | 240 |
| TCRBV02-01 CASSQDLWGSSQNTLYF TCRBJ02-04 | 241 |
| TCRBV04-01 CASSPTGEEQYF TCRBJ02-07 | 242 |
| TCRBV01-01 CTCSVTDSGNTLYF TCRBJ01-03 | 243 |
| TCRBV15-01 CASSLDNAETLYF TCRBJ02-03 | 244 |
| TCRBV01-01 CTCSAEGGRGEQYF TCRBJ02-07 | 245 |
| TCRBV13-03 CASSDWGEGEQYF TCRBJ02-07 | 246 |
| TCRBV13-03 CASSEDSGNTLYF TCRBJ01-03 | 247 |
| TCRBV13-01 CASSRGNSDYTF TCRBJ01-02 | 248 |
| TCRBV03-01 CASSSRDRGDSDYTF TCRBJ01-02 | 249 |
| TCRBV13-02 CASGGRYEQYF TCRBJ02-07 | 250 |
| TCRBV13-01 CASSDSGREQYF TCRBJ02-07 | 251 |
| TCRBV03-01 CASSLLGEQYF TCRBJ02-07 | 252 |
| TCRBV14-01 CASSRSYEQYF TCRBJ02-07 | 253 |
| TCRBV31-01 CAWSPRGNSDYTF TCRBJ01-02 | 254 |
| TCRBV01-01 CTCSADRGDYAEQFF TCRBJ02-01 | 255 |
| TCRBV01-01 CTCSAGTGGSNERLFF TCRBJ01-04 | 256 |
| TCRBV13-02 CASGDQGAGERLFF TCRBJ01-04 | 257 |
| TCRBV13-02 CASGDTGAGNTLYF TCRBJ01-03 | 258 |
| TCRBV13-02 CASGEGAYEQYF TCRBJ02-07 | 259 |
| TCRBV03-01 CASSATGGEQYF TCRBJ02-07 | 260 |
| TCRBV15-01 CASSDNYAEQFF TCRBJ02-01 | 261 |
| TCRBV29-01 CASSFGGANSDYTF TCRBJ01-02 | 262 |
| TCRBV12-01 CASSLKGSGNTLYF TCRBJ01-03 | 263 |
| TCRBV26-01 CASSLSLSNERLFF TCRBJ01-04 | 264 |
| TCRBV19-01 CASSPGQGAYEQYF TCRBJ02-07 | 265 |
| TCRBV04-01 CASSPLGGPYEQYF TCRBJ02-07 | 266 |
| TCRBV02-01 CASSQDWGLSYEQYF TCRBJ02-07 | 267 |
| TCRBV02-01 CASSQEGGGAYEQYF TCRBJ02-07 | 268 |
| TCRBV04-01 CASSRDSGNTLYF TCRBJ01-03 | 269 |
| TCRBV19-01 CASSRTGVYEQYF TCRBJ02-07 | 270 |
| TCRBV13-01 CASSDPGGTETLYF TCRBJ02-03 | 271 |
| TCRBV13-01 CASSDQGAYAEQFF TCRBJ02-01 | 272 |
| TCRBV13-01 CASSDRDTGQLYF TCRBJ02-02 | 273 |
| TCRBV14-01 CASSFTGDEQYF TCRBJ02-07 | 274 |
| TCRBV19-01 CASSMSYEQYF TCRBJ02-07 | 275 |
| TCRBV12-01 CASSPGDSGNTLYF TCRBJ01-03 | 276 |
| TCRBV16-01 CASSPGTGVNQAPLF TCRBJ01-05 | 277 |
| TCRBV02-01 CASSQDGQYAEQFF TCRBJ02-01 | 278 |
| TCRBV02-01 CASSQGLGVSYEQYF TCRBJ02-07 | 279 |
| TCRBV02-01 CASSRTGSAETLYF TCRBJ02-03 | 280 |
| TCRBV16-01 CASSSLSYEQYF TCRBJ02-07 | 281 |
| TCRBV20-01 CGAGTNNNQAPLF TCRBJ01-05 | 282 |
| TCRBV01-01 CTCSADLGSDYTF TCRBJ01-02 | 283 |
| TCRBV13-02 CASGVDSYEQYF TCRBJ02-07 | 284 |
| TCRBV13-03 CASSEGQGYAEQFF TCRBJ02-01 | 285 |
| TCRBV03-01 CASSFQGAYEQYF TCRBJ02-07 | 286 |
| TCRBV19-01 CASSGTTNSDYTF TCRBJ01-02 | 287 |
| TCRBV12-01 CASSLGGSNSDYTF TCRBJ01-02 | 288 |
| TCRBV26-01 CASSLSRNNQAPLF TCRBJ01-05 | 289 |
| TCRBV19-01 CASSMGRAGNTLYF TCRBJ01-03 | 290 |
| TCRBV15-01 CASSPDRNYAEQFF TCRBJ02-01 | 291 |
| TCRBV16-01 CASSPGQNERLFF TCRBJ01-04 | 292 |
| TCRBV15-01 CASSPGQSYEQYF TCRBJ02-07 | 293 |
| TCRBV16-01 CASSPTISNERLFF TCRBJ01-04 | 294 |
| TCRBV02-01 CASSQDGQGSYEQYF TCRBJ02-07 | 295 |
| TCRBV02-01 CASSQEQANSDYTF TCRBJ01-02 | 296 |
| TCRBV02-01 CASSQGHISNERLFF TCRBJ01-04 | 297 |
| TCRBV14-01 CASSYSQNTLYF TCRBJ02-04 | 298 |
| TCRBV19-01 CASTRDSSGNTLYF TCRBJ01-03 | 299 |
| TCRBV31-01 CAWSLPNSGNTLYF TCRBJ01-03 | 300 |
| TCRBV13-02 CASGDGRDEQYF TCRBJ02-07 | 301 |
| TCRBV13-02 CASGEGGNSGNTLYF TCRBJ01-03 | 302 |
| TCRBV13-02 CASGQGANERLFF TCRBJ01-04 | 303 |
| TCRBV13-03 CASRTTNSDYTF TCRBJ01-02 | 304 |
| TCRBV13-01 CASSDADRDEQYF TCRBJ02-07 | 305 |
| TCRBV13-01 CASSDARGRDTQYF TCRBJ02-05 | 306 |
| TCRBV04-01 CASSHRGGNQAPLF TCRBJ01-05 | 307 |
| TCRBV12-01 CASSLAGGGSYEQYF TCRBJ02-07 | 308 |
| TCRBV04-01 CASSLDISGNTLYF TCRBJ01-03 | 309 |
| TCRBV03-01 CASSLEGGDSDYTF TCRBJ01-02 | 310 |
| TCRBV16-01 CASSLGGPEQYF TCRBJ02-07 | 311 |
| TCRBV12-01 CASSLGGPYAEQFF TCRBJ02-01 | 312 |
| TCRBV12-02 CASSLTGGVEQYF TCRBJ02-07 | 313 |
| TCRBV26-01 CASSPGLGGSYEQYF TCRBJ02-07 | 314 |
| TCRBV02-01 CASSQDGVSGNTLYF TCRBJ01-03 | 315 |
| TCRBV05-01 CASSQEGGVEQYF TCRBJ02-07 | 316 |
| TCRBV16-01 CASSSGTGGGYEQYF TCRBJ02-07 | 317 |
| TCRBV31-01 CAWRQNSGNTLYF TCRBJ01-03 | 318 |
| TCRBV31-01 CAWSLGTNSGNTLYF TCRBJ01-03 | 319 |
| TCRBV31-01 CAWSLWGDEQYF TCRBJ02-07 | 320 |
| TCRBV01-01 CTCSAATNERLFF TCRBJ01-04 | 321 |
| TCRBV13-02 CASGARDNYAEQFF TCRBJ02-01 | 322 |
| TCRBV13-02 CASGAYAEQFF TCRBJ02-01 | 323 |
| TCRBV13-02 CASGDDTGGYEQYF TCRBJ02-07 | 324 |
| TCRBV13-02 CASGEQFF TCRBJ02-01 | 325 |
| TCRBV13-03 CASRDRNTGQLYF TCRBJ02-02 | 326 |
| TCRBV13-01 CASSDAVSQNTLYF TCRBJ02-04 | 327 |
| TCRBV13-01 CASSDLGDYAEQFF TCRBJ02-01 | 328 |
| TCRBV14-01 CASSFGGNTLYF TCRBJ01-03 | 329 |
| TCRBV04-01 CASSFQANSDYTF TCRBJ01-02 | 330 |
| TCRBV04-01 CASSFRNSDYTF TCRBJ01-02 | 331 |
| TCRBV12-02 CASSGGNYAEQFF TCRBJ02-01 | 332 |
| TCRBV13-03 CASSGGQGSAETLYF TCRBJ02-03 | 333 |
| TCRBV12-01 CASSHGLGGNYAEQFF TCRBJ02-01 | 334 |
| TCRBV16-01 CASSLAGRTEVFF TCRBJ01-01 | 335 |
| TCRBV03-01 CASSLDGGSYEQYF TCRBJ02-07 | 336 |
| TCRBV12-01 CASSLLGGREQYF TCRBJ02-07 | 337 |
| TCRBV03-01 CASSLLVNQDTQYF TCRBJ02-05 | 338 |
| TCRBV13-01 CASSLQGYEQYF TCRBJ02-07 | 339 |
| TCRBV19-01 CASSLRGSGNTLYF TCRBJ01-03 | 340 |
| TCRBV26-01 CASSLSVNSGNTLYF TCRBJ01-03 | 341 |
| TCRBV12-01 CASSLWGDEQYF TCRBJ02-07 | 342 |
| TCRBV12-02 CASSPTSSAETLYF TCRBJ02-03 | 343 |
| TCRBV02-01 CASSQDGQDTQYF TCRBJ02-05 | 344 |
| TCRBV05-01 CASSQEEGGEQYF TCRBJ02-07 | 345 |
| TCRBV02-01 CASSRDRGREQYF TCRBJ02-07 | 346 |
| TCRBV16-01 CASSRTTNSDYTF TCRBJ01-02 | 347 |
| TCRBV04-01 CASSSDRVGNTLYF TCRBJ01-03 | 348 |
| TCRBV16-01 CASSSGLGGENTLYF TCRBJ02-04 | 349 |
| TCRBV03-01 CASSSGTSNSDYTF TCRBJ01-02 | 350 |
| TCRBV31-01 CAWSLEGDTQYF TCRBJ02-05 | 351 |
| TCRBV31-01 CAWSLSGGARAEQFF TCRBJ02-01 | 352 |
| TCRBV20-01 CGARVGQNSDYTF TCRBJ01-02 | 353 |
| TCRBV01-01 CTCSAGGAPEQYF TCRBJ02-07 | 354 |
| TCRBV13-02 CASGDAGAEDTQYF TCRBJ02-05 | 355 |
| TCRBV13-02 CASGERLGVNQDTQYF TCRBJ02-05 | 356 |
| TCRBV13-02 CASGETGAQDTQYF TCRBJ02-05 | 357 |
| TCRBV13-03 CASRTSSAETLYF TCRBJ02-03 | 358 |
| TCRBV13-01 CASSDADIQDTQYF TCRBJ02-05 | 359 |
| TCRBV13-01 CASSDALNTEVFF TCRBJ01-01 | 360 |
| TCRBV13-03 CASSDRETLYF TCRBJ02-03 | 361 |
| TCRBV13-03 CASSDRGPNTGQLYF TCRBJ02-02 | 362 |
| TCRBV13-03 CASSERQNTLYF TCRBJ02-04 | 363 |
| TCRBV12-01 CASSGDSAETLYF TCRBJ02-03 | 364 |
| TCRBV19-01 CASSIGRNQDTQYF TCRBJ02-05 | 365 |
| TCRBV03-01 CASSLEGQNYAEQFF TCRBJ02-01 | 366 |
| TCRBV03-01 CASSLEGRNTGQLYF TCRBJ02-02 | 367 |
| TCRBV03-01 CASSLGFNQDTQYF TCRBJ02-05 | 368 |
| TCRBV12-02 CASSLGGAAETLYF TCRBJ02-03 | 369 |
| TCRBV12-01 CASSLGGGGAEQFF TCRBJ02-01 | 370 |
| TCRBV15-01 CASSLGTTNTGQLYF TCRBJ02-02 | 371 |
| TCRBV12-01 CASSLLGGRDTQYF TCRBJ02-05 | 372 |
| TCRBV03-01 CASSLLNQDTQYF TCRBJ02-05 | 373 |
| TCRBV12-02 CASSPDSSAETLYF TCRBJ02-03 | 374 |
| TCRBV03-01 CASSPDWGDTGQLYF TCRBJ02-02 | 375 |
| TCRBV02-01 CASSQAANTEVFF TCRBJ01-01 | 376 |
| TCRBV02-01 CASSQDHSSGNTLYF TCRBJ01-03 | 377 |
| TCRBV02-01 CASSQEGGRGAETLYF TCRBJ02-03 | 378 |
| TCRBV02-01 CASSQGRGAETLYF TCRBJ02-03 | 379 |
| TCRBV02-01 CASSQLGSSAETLYF TCRBJ02-03 | 380 |
| TCRBV02-01 CASSQPGANTEVFF TCRBJ01-01 | 381 |
| TCRBV04-01 CASSRDRNYAEQFF TCRBJ02-01 | 382 |
| TCRBV16-01 CASSRQGTEVFF TCRBJ01-01 | 383 |
| TCRBV31-01 CAWSLDTLYF TCRBJ02-04 | 384 |
| TCRBV01-01 CTCSAGDSPLYF TCRBJ01-06 | 385 |
| TCRBV01-01 CTCSAGQGADTEVFF TCRBJ01-01 | 386 |
| TCRBV01-01 CTCSAGVNSPLYF TCRBJ01-06 | 387 |
| TCRBV13-02 CASGDAGGTQDTQYF TCRBJ02-05 | 388 |
| TCRBV13-02 CASGDAGGVSQNTLYF TCRBJ02-04 | 389 |
| TCRBV13-02 CASGDAGRDTEVFF TCRBJ01-01 | 390 |
| TCRBV13-02 CASGDDWGGTGQLYF TCRBJ02-02 | 391 |
| TCRBV13-02 CASGDTGQNTLYF TCRBJ02-04 | 392 |
| TCRBV13-02 CASGEGTGGANTEVFF TCRBJ01-01 | 393 |
| TCRBV13-02 CASGQGASAETLYF TCRBJ02-03 | 394 |
| TCRBV13-03 CASRGTGDTEVFF TCRBJ01-01 | 395 |
| TCRBV13-03 CASSAGTTNTEVFF TCRBJ01-01 | 396 |
| TCRBV13-01 CASSDATGASQNTLYF TCRBJ02-04 | 397 |
| TCRBV04-01 CASSFTGGDTEVFF TCRBJ01-01 | 398 |
| TCRBV02-01 CASSHGQNTEVFF TCRBJ01-01 | 399 |
| TCRBV19-01 CASSKGQNTGQLYF TCRBJ02-02 | 400 |
| TCRBV03-01 CASSLASAETLYF TCRBJ02-03 | 401 |
| TCRBV03-01 CASSLDWGGREQYF TCRBJ02-07 | 402 |
| TCRBV03-01 CASSLEEDTQYF TCRBJ02-05 | 403 |
| TCRBV12-02 CASSLEGGSSYEQYF TCRBJ02-07 | 404 |
| TCRBV16-01 CASSLEGSSAETLYF TCRBJ02-03 | 405 |
| TCRBV04-01 CASSLGHNTEVFF TCRBJ01-01 | 406 |
| TCRBV12-01 CASSLGSYNSPLYF TCRBJ01-06 | 407 |
| TCRBV12-02 CASSLGTGSAETLYF TCRBJ02-03 | 408 |
| TCRBV16-01 CASSLGVQDTQYF TCRBJ02-05 | 409 |
| TCRBV19-01 CASSLRDWGNTGQLYF TCRBJ02-02 | 410 |
| TCRBV15-01 CASSLRGSAETLYF TCRBJ02-03 | 411 |
| TCRBV12-01 CASSLRVNQDTQYF TCRBJ02-05 | 412 |
| TCRBV29-01 CASSLSGQGNTEVFF TCRBJ01-01 | 413 |
| TCRBV03-01 CASSLVGDAETLYF TCRBJ02-03 | 414 |
| TCRBV19-01 CASSMGTTNTEVFF TCRBJ01-01 | 415 |
| TCRBV13-03 CASSPNTEVFF TCRBJ01-01 | 416 |
| TCRBV03-01 CASSPTGNTEVFF TCRBJ01-01 | 417 |
| TCRBV05-01 CASSQAGGASAETLYF TCRBJ02-03 | 418 |
| TCRBV02-01 CASSQEGGRNTLYF TCRBJ02-04 | 419 |
| TCRBV05-01 CASSQEGQGNSDYTF TCRBJ01-02 | 420 |
| TCRBV05-01 CASSQELGDYAEQFF TCRBJ02-01 | 421 |
| TCRBV02-01 CASSQGGGDTQYF TCRBJ02-05 | 422 |
| TCRBV05-01 CASSQRDTEVFF TCRBJ01-01 | 423 |
| TCRBV04-01 CASSRDWGGTGQLYF TCRBJ02-02 | 424 |
| TCRBV19-01 CASSRTGGDDTQYF TCRBJ02-05 | 425 |
| TCRBV19-01 CASSRTSSQNTLYF TCRBJ02-04 | 426 |
| TCRBV13-01 CASSVQGNTEVFF TCRBJ01-01 | 427 |
| TCRBV31-01 CAWSGQGANTEVFF TCRBJ01-01 | 428 |
| TCRBV31-01 CAWSLGDRGDERLFF TCRBJ01-04 | 429 |
| TCRBV31-01 CAWSLGGAEDTQYF TCRBJ02-05 | 430 |
| TCRBV20-01 CGARGTGGSDYTF TCRBJ01-02 | 431 |
| TCRBV20-01 CGASRNTEVFF TCRBJ01-01 | 432 |
| TCRBV01-01 CTCSADRGVEVFF TCRBJ01-01 | 433 |
| TCRBV01-01 CTCSAESSAETLYF TCRBJ02-03 | 434 |
| TCRBV01-01 CTCSAVGGDTQYF TCRBJ02-05 | 435 |

In various embodiments, the TARSs database comprising TCRβ sequences associated with smallpox vaccination is generated from a cohort of human subjects. Accordingly, in various embodiments, human TARSs associated with small pox vaccination can comprise any one of SEQ ID NOs: 436-674 (Table 3, below). As above, the TCRβ alleles are provided in IMTG nomenclature and identify the relevant human variable (V) and joining (J) segment that must be combined with the indicated CDR3 sequence to generate the relevant TCRβ allele. Nucleic acid and amino acid sequences for all of the human V and J regions used in this table can be obtained from the International ImMunoGenTics database is available (www.imgt.org).

TABLE 3
ACAM2000 Vaccine Associated TCR Library- Human

| | CDR3 |
|---|---|
| | SEQ ID |
| V-CDR3-J | NO: |
| TCRBV06-04\|CASSDGTTGELFF\|TCRBJ02-02*01 | 436 |
| TCRBV19-01\|CASSQHYEQYF\|TCRBJ02-07*01 | 437 |
| TCRBV28-01*01\|CASSFPRGSSYEQYF\|TCRBJ02-07*01 | 438 |
| TCRBV06-04\|CASSGTSGSTDTQYF\|TCRBJ02-03*01 | 439 |
| TCRBV06-04\|CASSDGTSGSNEQFF\|TCRBJ02-01*01 | 440 |
| TCRBV12\|CASSLSSNQPQHF\|TCRBJ01-05*01 | 441 |
| TCRBV12\|CASSLGGGETQYF\|TCRBJ02-05*01 | 442 |
| TCRBV18-01*01\|CASSPGPGNSYEQYF\|TCRBJ02-07*01 | 443 |
| TCRBV12\|CASSFTENTEAFF\|TCRBJ01-01*01 | 444 |
| TCRBV07-09\|CASSFGRGQETQYF\|TCRBJ02-05*01 | 445 |
| TCRBV04-03*01\|CASSQDGSPLHF\|TCRBJ01-06*01 | 446 |
| TCRBV18-01*01\|CASSPLSSYEQYF\|TCRBJ02-07*01 | 447 |
| TCRBV27-01*01\|CASSLRGNQPQHF\|TCRBJ01-05*01 | 448 |
| TCRBV27-01*01\|CASSLQGGNYGYTF\|TCRBJ01-02*01 | 449 |
| TCRBV19-01\|CASSIAARGNTEAFF\|TCRBJ01-01*01 | 450 |
| TCRBV20\|CSARQGDTEAFF\|TCRBJ01-01*01 | 451 |
| TCRBV28-01*01\|CASSLGGTEAFF\|TCRBJ01-01*01 | 452 |
| TCRBV07-09\|CASSLGRGGYGYTF\|TCRBJ01-02*01 | 453 |
| TCRBV07-08*01\|CASSLGTSASYEQYF\|TCRBJ02-07*01 | 454 |
| TCRBV19-01\|CASSMQGSTEAFF\|TCRBJ01-01*01 | 455 |
| TCRBV05-04*01\|CASSPTGDEQYF\|TCRBJ02-07*01 | 456 |
| TCRBV06\|CASRTVNQPQHF\|TCRBJ01-05*01 | 457 |
| TCRBV12\|CASSLAGTGGSGYTF\|TCRBJ01-02*01 | 458 |
| TCRBV27-01*01\|CASSLETNSYEQYF\|TCRBJ02-07*01 | 459 |
| TCRBV20\|CSAREGDTEAFF\|TCRBJ01-01*01 | 460 |
| TCRBV24\|CATIFQRGNQPQHF\|TCRBJ01-05*01 | 461 |
| TCRBV09-01\|CASSVTGGNEQFF\|TCRBJ02-01*01 | 462 |
| TCRBV29-01*01\|CSVGQDDYGYTF\|TCRBJ01-02*01 | 463 |
| TCRBV03\|CASSQAGTTYNEQFF\|TCRBJ02-01*01 | 464 |
| TCRBV19-01\|CASSIQGGTEAFF\|TCRBJ01-01*01 | 465 |
| TCRBV03\|CASRRQGNTEAFF\|TCRBJ01-01*01 | 466 |
| TCRBV19-01\|CASSRDPGRTEAFF\|TCRBJ01-01*01 | 467 |
| TCRBV05-01*01\|CASSLEGDQPQHF\|TCRBJ01-05*01 | 468 |
| TCRBV19-01\|CASSSRSSYEQYF\|TCRBJ02-07*01 | 469 |
| TCRBV20-01*01\|CSARERYEQYF\|TCRBJ02-07*01 | 470 |
| TCRBV10-03*01\|CAISGTSGTYEQYF\|TCRBJ02-07*01 | 471 |
| TCRBV06\|CASSWDGSNQPQHF\|TCRBJ01-05*01 | 472 |
| TCRBV19-01\|CASSTQGNTEAFF\|TCRBJ01-01*01 | 473 |
| TCRBV06\|CASSYGQENQPQHF\|TCRBJ01-05*01 | 474 |
| TCRBV06-01*01\|CASSGNRGGQPQHF\|TCRBJ01-05*01 | 475 |
| TCRBV09-01\|CASSVETGAETQYF\|TCRBJ02-05*01 | 476 |
| TCRBV04-03*01\|CASSQVLAGGSSYNEQFF\|TCRBJ02- | 477 |
| 01*01 | |
| TCRBV07-09\|CASSLGTASTDTQYF\|TCRBJ02-03*01 | 478 |
| TCRBV06-01*01\|CASSSQGGTEAFF\|TCRBJ01-01*01 | 479 |
| TCRBV06-05*01\|CASRRGVNQPQHF\|TCRBJ01-05*01 | 480 |
| TCRBV27-01*01\|CASSYEGPYEQYF\|TCRBJ02-07*01 | 481 |
| TCRBV27-01*01\|CASSFEGAYEQYF\|TCRBJ02-07*01 | 482 |
| TCRBV06\|CASSSTGELFF\|TCRBJ02-02*01 | 483 |
| TCRBV05-01*01\|CASSLVGEQYF\|TCRBJ02-07*01 | 484 |
| TCRBV03\|CASSRDSNQPQHF\|TCRBJ01-05*01 | 485 |
| TCRBV06-05*01\|CASSYGGRQPQHF\|TCRBJ01-05*01 | 486 |
| TCRBV06-04\|CASSDSSGANVLTF\|TCRBJ02-06*01 | 487 |
| TCRBV27-01*01\|CASSLEGYEQYF\|TCRBJ02-07*01 | 488 |
| TCRBV07-02*01\|CASSLRYEQYF\|TCRBJ02-07*01 | 489 |
| TCRBV02-01*01\|CASSRGDNQPQHF\|TCRBJ01-05*01 | 490 |
| TCRBV07-02*01\|CASSLRRGTDTQYF\|TCRBJ02-03*01 | 491 |
| TCRBV04-01*01\|CASSQGGEETQYF\|TCRBJ02-05*01 | 492 |
| TCRBV07-06*01\|CASSPGTSYEQYF\|TCRBJ02-07*01 | 493 |
| TCRBV12\|CASSSTSTDTQYF\|TCRBJ02-03*01 | 494 |
| TCRBV05-01*01\|CASSLEYGYEQYF\|TCRBJ02-07*01 | 495 |
| TCRBV29-01*01\|CSVLDNGYTF\|TCRBJ01-02*01 | 496 |
| TCRBV06-01*01\|CASSEGQSYEQYF\|TCRBJ02-07*01 | 497 |
| TCRBV07-02*01\|CASSFTGSPGQEQYF\|TCRBJ02-07*01 | 498 |
| TCRBV20\|CSARDRTGNGYTF\|TCRBJ01-02*01 | 499 |
| TCRBV20\|CSARQDSNQPQHF\|TCRBJ01-05*01 | 500 |
| TCRBV04-03*01\|CASSQDRAGGTEAFF\|TCRBJ01-01*01 | 501 |
| TCRBV02-01*01\|CASSVGAGTEAFF\|TCRBJ01-01*01 | 502 |
| TCRBV03\|CASSQGDQGAKNIQYF\|TCRBJ02-04*01 | 503 |
| TCRBV27-01*01\|CASSFEGPYEQYF\|TCRBJ02-07*01 | 504 |
| TCRBV06-04\|CASSDSTSGSNEQFF\|TCRBJ02-01*01 | 505 |
| TCRBV02-01*01\|CASSEGQVWPGELFF\|TCRBJ02-02*01 | 506 |
| TCRBV06-04\|CASSDSDTGELFF\|TCRBJ02-02*01 | 507 |
| TCRBV04-01*01\|CASSLEGDLSGNTIYF\|TCRBJ01-03*01 | 508 |
| TCRBV06\|CASSYSSGANVLTF\|TCRBJ02-06*01 | 509 |
| TCRBV05-01*01\|CASSLVVQPYEQYF\|TCRBJ02-07*01 | 510 |
| TCRBV20-01*01\|CSASGRETQYF\|TCRBJ02-05*01 | 511 |
| TCRBV28-01*01\|CASSGVYGYTF\|TCRBJ01-02*01 | 512 |
| TCRBV19-01\|CASSPQGGYGYTF\|TCRBJ01-02*01 | 513 |
| TCRBV06\|CASSYSGQGFEQYF\|TCRBJ02-07*01 | 514 |
| TCRBV05-04*01\|CASSLDADLQYF\|TCRBJ02-03*01 | 515 |
| TCRBV12\|CASSLQGMNTEAFF\|TCRBJ01-01*01 | 516 |
| TCRBV20\|CSARGGIPYEQYF\|TCRBJ02-07*01 | 517 |
| TCRBV24\|CATSDRTGGNEQYF\|TCRBJ02-07*01 | 518 |
| TCRBV30-01*01\|CAWSRQGGNQPQHF\|TCRBJ01-05*01 | 519 |
| TCRBV19-01\|CASSIEGARTEAFF\|TCRBJ01-01*01 | 520 |
| TCRBV05-04*01\|CASSLDRSYEQYF\|TCRBJ02-07*01 | 521 |
| TCRBV09-01\|CASSVGSGGSSTDTQYF\|TCRBJ02-03*01 | 522 |
| TCRBV09-01\|CASSVTGGYEQYF\|TCRBJ02-07*01 | 523 |
| TCRBV19-01\|CASSIRGGNTEAFF\|TCRBJ01-01*01 | 524 |
| TCRBV02-01*01\|CASSAWRGGFHEQYF\|TCRBJ02-07*01 | 525 |
| TCRBV10-01\|CASSEGQGTYEQYF\|TCRBJ02-07*01 | 526 |
| TCRBV24\|CATSDSRITEQFF\|TCRBJ02-01*01 | 527 |
| TCRBV04-01*01\|CASSLEAARNQPQHF\|TCRBJ01-05*01 | 528 |
| TCRBV06\|CASRPGQGQPQHF\|TCRBJ01-05*01 | 529 |
| TCRBV19-01\|CASSLQGNTEAFF\|TCRBJ01-01*01 | 530 |
| TCRBV07-06*01\|CASSLGETQYF\|TCRBJ02-05*01 | 531 |
| TCRBV12\|CASSLRGYGYTF\|TCRBJ01-02*01 | 532 |
| TCRBV09-01\|CASSVTTGYEQYF\|TCRBJ02-07*01 | 533 |
| TCRBV20\|CSAGLAGGTPDTQYF\|TCRBJ02-03*01 | 534 |
| TCRBV27-01*01\|CASSLRGSSYEQYF\|TCRBJ02-07*01 | 535 |
| TCRBV27-01*01\|CASSLEGPYEQYF\|TCRBJ02-07*01 | 536 |
| TCRBV07-09\|CASSFGRGNTEAFF\|TCRBJ01-01*01 | 537 |
| TCRBV30-01*01\|CAWSLKGDSPLHF\|TCRBJ01-06*01 | 538 |
| TCRBV06\|CASSYSDTYEQYF\|TCRBJ02-07*01 | 539 |
| TCRBV03\|CASSQGGNTEAFF\|TCRBJ01-01*01 | 540 |
| TCRBV06-06\|CASSYRDSNQPQHF\|TCRBJ01-05*01 | 541 |
| TCRBV28-01*01\|CASSLWGTSTDTQYF\|TCRBJ02-03*01 | 542 |
| TCRBV07-08*01\|CASSLGQTYNSPLHF\|TCRBJ01-06*01 | 543 |
| TCRBV24\|CATSEGQGAVGYTF\|TCRBJ01-02*01 | 544 |
| TCRBV07-02*01\|CASSPEGQAAGYTF\|TCRBJ01-02*01 | 545 |
| TCRBV12\|CASSLTSTDTQYF\|TCRBJ02-03*01 | 546 |
| TCRBV05-04*01\|CASSLAAGSGNTIYF\|TCRBJ01-03*01 | 547 |
| TCRBV19-01\|CASSIRSAYEQYF\|TCRBJ02-07*01 | 548 |
| TCRBV09-01\|CASSLTGGYEQYF\|TCRBJ02-07*01 | 549 |
| TCRBV06\|CASSYSTSGYEQYF\|TCRBJ02-07*01 | 550 |
| TCRBV05-06*01\|CASSLASGWYEQYF\|TCRBJ02-07*01 | 551 |
| TCRBV04-01*01\|CASSRGTGDTEAFF\|TCRBJ01-01*01 | 552 |
| TCRBV06-04\|CASSDGQGADTQYF\|TCRBJ02-03*01 | 553 |
| TCRBV24\|CATSDGQGEVGYTF\|TCRBJ01-02*01 | 554 |
| TCRBV09-01\|CASSATGGNQPQHF\|TCRBJ01-05*01 | 555 |
| TCRBV19-01\|CASSIQGNTEAFF\|TCRBJ01-01*01 | 556 |
| TCRBV20\|CSASRESDTQYF\|TCRBJ02-03*01 | 557 |
| TCRBV04-01*01\|CASSQGDRGYGYTF\|TCRBJ01-02*01 | 558 |
| TCRBV27-01*01\|CASSPTGSSYEQYF\|TCRBJ02-07*01 | 559 |
| TCRBV09-01\|CASSVDSLNYGYTF\|TCRBJ01-02*01 | 560 |
| TCRBV19-01\|CASSVRSSYEQYF\|TCRBJ02-07*01 | 561 |
| TCRBV27-01*01\|CASSLETNTGELFF\|TCRBJ02-02*01 | 562 |
| TCRBV27-01*01\|CASSLEGGYEQYF\|TCRBJ02-07*01 | 563 |
| TCRBV20\|CSARLAGGQETQYF\|TCRBJ02-05*01 | 564 |
| TCRBV24\|CATSEGQGDVGYTF\|TCRBJ01-02*01 | 565 |
| TCRBV03\|CASSHSYEQYF\|TCRBJ02-07*01 | 566 |
| TCRBV07-02*01\|CASSLPSAGGYTF\|TCRBJ01-02*01 | 567 |
| TCRBV06-04\|CASSDSNTGELFF\|TCRBJ02-02*01 | 568 |
| TCRBV03\|CASSPGLAGDEQYF\|TCRBJ02-07*01 | 569 |
| TCRBV02-01*01\|CASSVGDNQPQHF\|TCRBJ01-05*01 | 570 |
| TCRBV27-01*01\|CASSLSSNQPQHF\|TCRBJ01-05*01 | 571 |
| TCRBV10-01\|CASSPGYEQYF\|TCRBJ02-07*01 | 572 |
| TCRBV20\|CSARGRAYNQPQHF\|TCRBJ01-05*01 | 573 |
| TCRBV06-05*01\|CASSPGQGRYEQYF\|TCRBJ02-07*01 | 574 |
| TCRBV04-03*01\|CASSQDGFNQPQHF\|TCRBJ01-05*01 | 575 |
| TCRBV27-01*01\|CASSLETNTEAFF\|TCRBJ01-01*01 | 576 |
| TCRBV27-01*01\|CASSFRNQPQHF\|TCRBJ01-05*01 | 577 |
| TCRBV03\|CASSQAGGTEAFF\|TCRBJ01-01*01 | 578 |
| TCRBV18-01*01\|CASSPGQVNTGELFF\|TCRBJ02-02*01 | 579 |
| TCRBV10-02*01\|CASSESTGYNQPQHF\|TCRBJ01-05*01 | 580 |
| TCRBV03\|CASSQQGADTQYF\|TCRBJ02-03*01 | 581 |
| TCRBV06-01*01\|CASSATGSYGYTF\|TCRBJ01-02*01 | 582 |
| TCRBV07-09\|CASSLGRGPYGYTF\|TCRBJ01-02*01 | 583 |
| TCRBV07-08*01\|CASSLRGGERGNTIYF\|TCRBJ01-03*01 | 584 |
| TCRBV27-01*01\|CASSPEGPYEQYF\|TCRBJ02-07*01 | 585 |
| TCRBV04-01*01\|CASSHQPGDYEQYF\|TCRBJ02-07*01 | 586 |
| TCRBV27-01*01\|CASSSGTYNEQFF\|TCRBJ02-01*01 | 587 |
| TCRBV10-02*01\|CASSESPGNSNQPQHF\|TCRBJ01-05*01 | 588 |
| TCRBV27-01*01\|CASSGGRDYGYTF\|TCRBJ01-02*01 | 589 |
| TCRBV29-01*01\|CSVGTGGTNEKLFF\|TCRBJ01-04*01 | 590 |
| TCRBV03\|CASSRTGELFF\|TCRBJ02-02*01 | 591 |
| TCRBV06\|CASSPPPGTGADTQYF\|TCRBJ02-03*01 | 592 |
| TCRBV15-01*01\|CATSRDSSGANVLTF\|TCRBJ02-06*01 | 593 |
| TCRBV06\|CASSYSRQGDGYTF\|TCRBJ01-02*01 | 594 |
| TCRBV04-02*01\|CASSQGWSSGGYEQYF\|TCRBJ02-07*01 | 595 |
| TCRBV05-05*01\|CASSLVDSVGYTF\|TCRBJ01-02*01 | 596 |
| TCRBV07-02*01\|CASSSPRGSSYEQYF\|TCRBJ02-07*01 | 597 |
| TCRBV06-05*01\|CASNQQGSTEAFF\|TCRBJ01-01*01 | 598 |
| TCRBV30-01*01\|CAWSVMGNYGYTF\|TCRBJ01-020l | 599 |
| TCRBV04-02*01\|CASSQAGTGVYEQYF\|TCRBJ02-07*01 | 600 |
| TCRBV06-01*01\|CASSEGTSGSYEQYF\|TCRBJ02-07*01 | 601 |
| TCRBV28-01*01\|CASSLSYEQYF\|TCRBJ02-07*01 | 602 |
| TCRBV06-05*01\|CASSYSTGEAFF\|TCRBJ01-01*01 | 603 |
| TCRBV12\|CASSLTGAYNEQFF\|TCRBJ02-01*01 | 604 |
| TCRBV05-01*01\|CASSLGQGNYGYTF\|TCRBJ01-02*01 | 605 |
| TCRBV02-01*01\|CASSGDGNYGYTF\|TCRBJ01-02*01 | 606 |
| TCRBV06-04\|CASSDNSGANVLTF\|TCRBJ02-06*01 | 607 |
| TCRBV11-02*02\|CASSLAGGTEAFF\|TCRBJ01-01*01 | 608 |
| TCRBV03\|CASSPAGGTEAFF\|TCRBJ01-01*01 | 609 |
| TCRBV19-01\|CASSIGTDTQYF\|TCRBJ02-03*01 | 610 |
| TCRBV07-09\|CASSLGGGEAFF\|TCRBJ01-01*01 | 611 |
| TCRBV27-01*01\|CASSLEGYGYTF\|TCRBJ01-02*01 | 612 |
| TCRBV27-01*01\|CASSLEGGNTEAFF\|TCRBJ01-01*01 | 613 |
| TCRBV30-01*01\|CAWSGQGGNQPQHF\|TCRBJ01-05*01 | 614 |
| TCRBV05-06*01\|CASRAGGYYGYTF\|TCRBJ01-02*01 | 615 |
| TCRBV30-01*01\|CAWRGQGGNQPQHF\|TCRBJ01-05*01 | 616 |
| TCRBV06-05*01\|CASRHRDSYEQYF\|TCRBJ02-07*01 | 617 |
| TCRBV06\|CASSYSERSEQFF\|TCRBJ02-01*01 | 618 |
| TCRBV19-01\|CASSIQGSTEAFF\|TCRBJ01-0101 | 619 |
| TCRBV06-01*01\|CASRQGSYEQYF\|TCRBJ02-07*01 | 620 |
| TCRBV04-03*01\|CASGRDISTDTQYF\|TCRBJ02-03*01 | 621 |
| TCRBV20-01*01\|CSARDGYEQYF\|TCRBJ02-07*01 | 622 |
| TCRBV19-01\|CASSRAARGNTEAFF\|TCRBJ01-01*01 | 623 |
| TCRBV02-01*01\|CASSTGDNQPQHF\|TCRBJ01-05*01 | 624 |
| TCRBV18-01*01\|CASSQLVGPYSPLHF\|TCRBJ01-06*01 | 625 |
| TCRBV06-04\|CASSDRGTGELFF\|TCRBJ02-02*01 | 626 |
| TCRBV05-01*01\|CASSPGTANTEAFF\|TCRBJ01-01*01 | 627 |
| TCRBV18-01*01\|CASSPGTANTGELFF\|TCRBJ02-02*01 | 628 |
| TCRBV07-08*01\|CASSLGQAYEQYF\|TCRBJ02-07*01 | 629 |
| TCRBV06\|CASSYSKTGGSNQPQHF\|TCRBJ01-05*01 | 630 |
| TCRBV09-01\|CASSVENYGYTF\|TCRBJ01-02*01 | 631 |
| TCRBV02-01*01\|CASRVQGLGNQPQHF\|TCRBJ01-05*01 | 632 |
| TCRBV04-03*01\|CASSQDKGGTEAFF\|TCRBJ01-01*01 | 633 |
| TCRBV02-01*01\|CASSGDTF\|TCRBJ01-02*01 | 634 |
| TCRBV06-05*01\|CASSPTGPEQYF\|TCRBJ02-07*01 | 635 |
| TCRBV28-01*01\|CASSPGQGVNYGYTF\|TCRBJ01-02*01 | 636 |
| TCRBV20\|CSARDDRGSYNEQFF\|TCRBJ02-01*01 | 637 |
| TCRBV19-01\|CASSIIGASNQPQHF\|TCRBJ01-05*01 | 638 |
| TCRBV27-01*01\|CASSLSGNSPLHF\|TCRBJ01-06*01 | 639 |
| TCRBV27-01*01\|CASSFETYNEQFF\|TCRBJ02-01*01 | 640 |
| TCRBV11-02*02\|CASSLAGHQPQHF\|TCRBJ01-05*01 | 641 |
| TCRBV27-01*01\|CASSFETNTGELFF\|TCRBJ02-02*01 | 642 |
| TCRBV02-01*01\|CASSVGGGYTF\|TCRBJ01-02*01 | 643 |
| TCRBV09-01\|CASSVGWGNTEAFF\|TCRBJ01-01*01 | 644 |
| TCRBV04-03*01\|CASSPQRNTEAFF\|TCRBJ01-01*01 | 645 |
| TCRBV04-03*01\|CASSQDRTGPEQYF\|TCRBJ02-07*01 | 646 |
| TCRBV10-01\|CASSESQGNTEAFF\|TCRBJ01-01*01 | 647 |
| TCRBV06-04\|CASSDGTSGYNEQFF\|TCRBJ02-01*01 | 648 |
| TCRBV18-01*01\|CASSPGQGGQPQHF\|TCRBJ01-0501 | 649 |
| TCRBV02-01*01\|CAGGGQYF\|TCRBJ02-07*01 | 650 |
| TCRBV19-01\|CASSIRSSYEQYF\|TCRBJ02-07*01 | 651 |
| TCRBV06-04\|CASSDRDTGELFF\|TCRBJ02-02*01 | 652 |
| TCRBV07-02*01\|CASSLDRVGTEAFF\|TCRBJ01-01*01 | 653 |
| TCRBV03\|CASSQEGRNTEAFF\|TCRBJ01-01*01 | 654 |
| TCRBV19-01\|CASSIAGIYNSPLHF\|TCRBJ01-06*01 | 655 |
| TCRBV03\|CASSPGTASGNTIYF\|TCRBJ01-03*01 | 656 |
| TCRBV24\|CATSEGQGETEAFF\|TCRBJ01-01*01 | 657 |
| TCRBV05-01*01\|CASSLRGSSYEQYF\|TCRBJ02-07*01 | 658 |
| TCRBV05-01*01\|CASSLVVSPYEQYF\|TCRBJ02-07*01 | 659 |
| TCRBV02-01*01\|CASGTGDNQPQHF\|TCRBJ01-05*01 | 660 |
| TCRBV27-01*01\|CASSLQGANYEQYF\|TCRBJ02-07*01 | 661 |
| TCRBV11-02*02\|CASSLGRTIYF\|TCRBJ01-03*01 | 662 |
| TCRBV19-01\|CASSIQGDTEAFF\|TCRBJ01-01*01 | 663 |
| TCRBV06\|CASSYGTNSYEQYF\|TCRBJ02-07*01 | 664 |
| TCRBV09-01\|CASSVTPGQGHEQYF\|TCRBJ02-07*01 | 665 |
| TCRBV07-09\|CASSLGRGNTEAFF\|TCRBJ01-01*01 | 666 |
| TCRBV20\|CSARDGNQPQHF\|TCRBJ01-05*01 | 667 |
| TCRBV07-09\|CASSLDSPNYGYTF\|TCRBJ01-02*01 | 668 |
| TCRBV06-05*01\|CASSPRGRGNQPQHF\|TCRBJ01-05*01 | 669 |
| TCRBV05-01*01\|CASSSGQPNTEAFF\|TCRBJ01-01*01 | 670 |
| TCRBV07-02*01\|CASSLQGAWGELFF\|TCRBJ02-02*01 | 671 |
| TCRBV07-02*01\|CASSFLAGAREQYF\|TCRBJ02-07*01 | 672 |
| TCRBV12-05*01\|CASGLFHEQYF\|TCRBJ02-07*01 | 673 |
| TCRBV24\|CATSDLVGTGGTGELFF\|TCRBJ02-02*01 | 674 |

### Method of Testing the Efficacy of a Vaccine

A method of testing the efficacy of a vaccine is also provided. In various embodiments, a vaccine is considered “effective” if it stimulates a robust immune response. For instance, an effective vaccine would be expected to stimulate T-cell expansion and antibody generation against an immunogenic antigen comprised by the vaccine. The methods provided herein can test the efficacy of a vaccine by identifying TCRβ sequences in the subject that are associated with the vaccination.

In various embodiments, the method of testing the efficacy of a vaccine comprises: (a) amplifying and sequencing TCRβ alleles in mRNA and/or genomic DNA of T-cells obtained from a subject after administration of the vaccine; (b) comparing the TCRβ clonotype profile of the subject to a database of vaccine associated TCRβ sequences (VATSs) statistically associated with vaccination to generate a diagnostic classifier of the subject, wherein the diagnostic classifier comprises the number of VATSs identified in the subject relative to the total number of unique TCRβ alleles in the subject; and (c) determining that the vaccine is effective in generating an immune response if the diagnostic classifier exceeds a threshold determined by the prevalence of VATSs in an independent test cohort after exposure to the vaccine.

In various embodiments, the method of testing the efficacy of the vaccine can further comprise administering the vaccine to the subject.

In various embodiments, the vaccine tested can comprise an Orthopoxvirus vaccine (e.g., the smallpox vaccine) a coronavirus vaccine (e.g., a SARS-CoV-2 vaccine) or an influenza vaccine (e.g., Influenza A or Influenza B vaccine) In various embodiments, the vaccine can comprise the smallpox vaccine and a TCRβ allele associated with the vaccination can comprise any one of SEQ ID NOs: 122-435.

### Method of Identifying a Viral Infection in a Subject

Also provided are methods of detecting/identifying a viral infection in a subject. In various embodiments, the method can comprise: (a) amplifying and sequencing TCRβ alleles in mRNA and/or genomic DNA of T-cells obtained from the subject; (b) comparing the TCRβ sequences in the subject to one or more databases of virus-associated TCRβ sequences, wherein each database comprises TCRβ sequences statistically associated with one virus and each database is generated according to the methods described above; and (c) identifying the viral infection of the subject by determining the strength of the association of the TCRβ allele sequences identified in the subject to one or more of the databases.

In various embodiments, the strength of the association can comprise performing a probability distribution function to determine whether the TCRβ clonotype profile of the subject is statistically similar to the TCRβ clonotype distribution in naïve or virus infected samples.

Advantageously, the method described herein can be used to distinguish between viruses that present with similar symptoms and etiology but stimulate clonal expansion of different T cell populations.

In various embodiments, the viral infection can comprise a smallpox infection. In various embodiments, the method can distinguish between a smallpox virus and a Zika virus.

In various embodiments, the viral infection can comprise a coronavirus infection. Exemplary coronaviruses include Severe Acute Respiratory Syndrome coronaviruses (e.g., SARS, including the new SARS-CoV-2 strain) and Middle Eastern Respiratory Syndrome (MERS) coronavirus. One useful application for this method is to identify individuals infected with SARS-CoV-2 (i.e., COVID-19). In additional embodiments, the viral infection can comprise influenza (e.g., Influenza A or Influenza B). For example, the influenza virus can comprise an H1N1 Influenza A strain.

In various embodiments, when the methods provided herein comprise analyzing a sample obtained from a subject, the subject can be a mammal. In various embodiments, the subject is a mouse. In other embodiments, the subject is a human.

The immune repertoire of a human is orders of magnitude larger than that of a mouse (particularly a model organism kept in immune privileged conditions and genetically identical to other subjects). This presents unique challenges in generating the TARSs database of TCRβ sequences associated with an immune response in humans.

Accordingly, a method is also provided for generating a TCRβ database comprising TCRβ sequences statistically associated with an immune condition, exposure to a vaccine or immunogenic agent and/or pathogen. The method comprises: (a) amplifying and sequencing TCRβ alleles in mRNA and/or genomic DNA of T-cells obtained from a cohort of subjects having the immune condition, or having been exposed to the vaccine, immunogenic agent and/or pathogen; and (b) using a machine learning and/or deep neural network system to analyze the TCRβ allele sequences and statistically associate a subset of the TCRβ sequences to the immune condition, vaccine, immunogenic agent and/or pathogen. The machine learning and/or deep neural network can perform the Fisher exact tests described above, or may perform different statistical tests.

### Deep Neural Network (DNN) Learning Algorithms

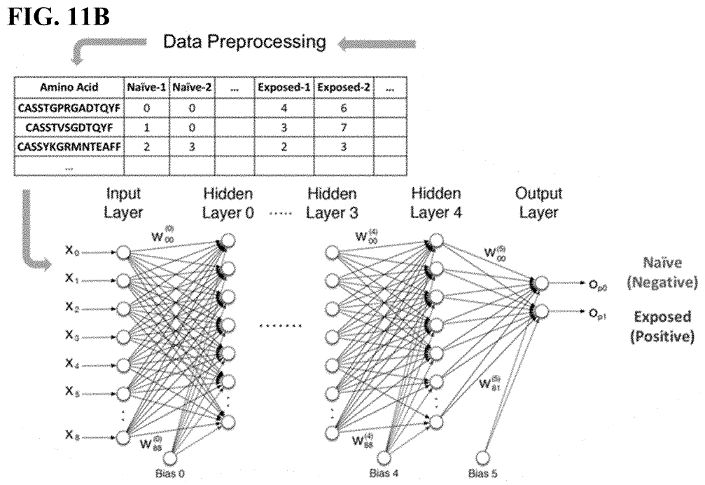

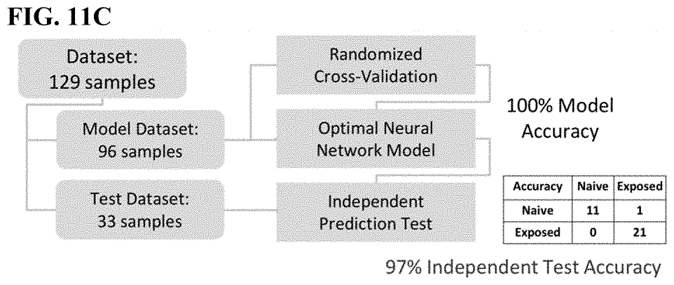

As one of the most powerful machine learning methods, the deep learning neural network has been substantially employed to explore the high-level features hidden in biomedical data. As provided herein, the deep learning framework is used to train the deep learning models for diagnostic discrimination. A multi-layer neural network is used to extract hidden patterns from the input features through differing numbers of hidden layers (i.e., using more than three layers, more than four layers, or more than five layers, etc.). The extracted hidden features are finally fed into the last layer of logistic regression to classify the sample into binary classes. See FIG. 11B The deep learning model can be optimized through minimizing the binary cross-entropy objective function in the process of standard error backward propagation. In addition, several parameters of neural networks can be adjusted when cross-validating the network, including the number of hidden layers, the number of hidden nodes in each hidden layer, and the types of activation functions for the hidden nodes. In various embodiments, for example, the neural network may comprise more than three hidden layers, more than four hidden layers or more than five hidden layers. In various embodiments, the neural network can comprise about three, about four or about five hidden layers. Several hyper-parameters can also be tuned, including the dropout rate for regularization, learning rate and momentum used in different types of optimization algorithms. The classification accuracy is calculated for each round of five-fold cross-validation, and the accuracy scores are averaged over a total of 50 rounds to select the best parameter set for final testing. In various embodiments, the deep neural network can be implemented using the Tensorflow library (www.tensorflow.org), along with the cross-validation and parameter tuning available in the Scikit-learn library.

### Model Training

In this work, the predictive ability of the DNN method for diagnostic discrimination of viral infection are evaluated to understand how immune system features can diagnose viral infection status. The frequency counts of all CDR3 amino acid sequences (a.k.a. peptides) can be calculated from quantified TCR beta chain sequence data and used as input features for machine learning methods to build discriminative classifiers. Each negative sample (pre-inoculation) or positive sample (post-inoculation) can be described as a vector of frequency counts, each representing the number of CDR3 amino acids found in the sequence data of the sample.

The analysis starts with the data partition. A stratified sampling method can be used to randomly divide the data into subsets according to the status of infection (pre- or post-introduction). The datasets can then be further partitioned into a training set and a testing set. In various embodiments, the training set can comprise at least about 50%, at least 60%, at least 70%, or at least 75%, of the data. For example, the training set can comprise about 50% to 90%, about 50% to 80%, about 50% to 75%, about 60% to 90%, about 60% to 80%, or about 70% to 80% of the data. Data not incorporated into the training set can be reserved to test the model (e.g., see FIG. 11C). In an example, the data can be divided with a ratio of 75%/25% (training/testing).

Once the data is divided, a repeated multi-fold cross-validation can be used to estimate the optimal parameters of each machine learning algorithm on the training dataset. The best training parameters selected by cross-validation are used to retrain the whole training dataset to derive the final model for evaluation. The independent testing subset is only seen when the final model of each algorithm is determined. After the training data is collected, several data normalization schemes are attempted before applying machine learning algorithms for model learning. Due to the different experimental conditions (i.e., sequence depth) and sample variations, the number of frequency counts for amino acids might vary in magnitude. Normalization might be necessary to remove inherent bias for different machine learning methods. For example, the data may be transformed using (1) peptide-based normalization that normalizes counts across all training samples within each amino acid sequence; (2) sample-based normalization that normalizes counts of amino acids within individual samples; and/or (3) the benchmark data that uses original counts without any normalization. The Minimum-Maximum transformation is adopted to convert counts into the range between zero and one when the normalization is needed. The normalized/original features are then used to train different machine learning models for infection diagnosis.

As a nonlimiting example, the model may comprise 5 hidden layers, 90 nodes (neurons), and 1000 max iterations. The model can show high prediction accuracy (e.g., greater than 80%, greater than 85%, greater than 90%, greater than 95%, greater than 96% or about 97%) when tested on previously unseen (independent test) samples while retaining 100% accuracy when identifying previously seen (training) samples. In addition, the parameters can be properly configured by randomized hyper-parameter search strategy since the DNN algorithm may affect proposed model's effectiveness. If inappropriate parameters are selected, the weights or coefficients in the deep neural network do not converge, rendering the trained models unusable.

The accuracy of this protocol in humans, which have significantly more genetic diversity than lab mice, can be improved by increasing accuracy of the sequencing of TCRβ alleles in the T-cells of the population. One way to increase sequencing accuracy, provided herein, is to use an ultra-deep sequencing protocol. In ultra-deep sequencing, the number of independent reads of a given sequence often exceed 1 million or even 2, 3, 4 or 5 million. Accordingly, in various embodiments, any steps provided herein that require amplifying and sequencing TCRβ alleles may be performed using an ultra-deep sequencing protocol. In various embodiments, the sequencing of the TCRβ alleles is performed at a depth of at least about 2 million, at least about 3 million, or at least about 5 million reads. For example, the sequencing of the TCRβ alleles can be performed at a depth of from about 2 million to about 100 million reads, from about 2 million to about 10 million reads, from about 2 million to about 5 million reads, from about 4 million to about 100 million reads, from about 4 million to about 10 million reads, from about 4 million to about 6 million reads, or from about 4 million to about 5 million reads.

Having described the invention in detail, it will be apparent that modifications and variations are possible without departing from the scope of the invention defined in the appended claims.

### EXAMPLES

The following non-limiting examples are provided to further illustrate the present invention.

### Materials and Methods

Materials and reagents used in the following examples are provided in Table 4 below. Common methods also used are provided herein below.

TABLE 4

| | | |
|---|---|---|
| Reagent or Resource | Source | Identifier |

Antibodies

| | | |
|---|---|---|
| anti-mouse IgG- | Sigma-Aldrich | RRID: AB_258426 |
| Peroxidase | | |
| anti-mouse CD4 | ebioscience | RRID: AB_11149869 |
| PerCP-efluor710 | | |
| (Clone GK1.5) | | |
| anti-mouse CD8 | BD Horizon | RRID: AB_1645281 |
| BV450 | | |
| (Clone 53-6.7) | | |
| anti-mouse CD19 | BD Horizon | RRID: AB_2732057 |
| BV605 (clone ID3) | | |

Bacterial and Virus Strains

| | | |
|---|---|---|
| ACAM2000 smallpox | CDC | NA |
| vaccine | | |
| MPXV ZAI-79 | Stabenow et. al. (2010) | NA |

Chemicals, Peptides and Recombinant Proteins

| | | |
|---|---|---|
| HLA-A2.1 Tetramer-PE | Gilchuk et. al. (2013) | NA |
| ACK Lysis Buffer | Lonza | www.lonza.com |
| Fetal Bovine Serum | Sigma-Aldrich | Cat# N4637 |
| RPMI-1640 | Fisher | Cat# MT15040CV |

Critical Commercial Assays

| | | |
|---|---|---|
| QIAGEN Blood and | QIAGEN | Cat # 69506 |
| Tissue Kit | | |
| ImmunoSeq | Adaptive | RRID: SCR_014709 |
| | Biotechnologies | |

Experimental Models: Cell Lines

| | | |
|---|---|---|
| BSC-1 cells | ATCC | RRID: CVCL_0607 |

Experimental Models: Organisms/Strains

| | | |
|---|---|---|
| Mouse: AAD C57BL/6J | The Jackson Laboratory | RRID: IMSR_JAX: 004191 |

Software and Algorithms

| | | |
|---|---|---|
| FlowJo 7.5 | TreeStar Inc. | RRID: SCR_008520 |
| GraphPad Prism 5.0 | GraphPad Software, Inc. | RRID: SCR_002798 |
| ImmunoSeq Analyzer | Adaptive | RRID: SCR_014709 |
| | Biotechnologies | |

Other

| | | |
|---|---|---|
| BD FACSAria II cell | BD Biosciences | NA |
| sorter | | |
| ImmunoSeq System | Illumina | NA |

### Mice

HLA-A2.1 AAD C57BL/6 male and female mice were purchased from Jackson Laboratories and maintained under specific pathogen conditions (Gilchuck et al., 2013). The HLA-A2.1 AAD mice express a transgenic HLA-A2.1 chimeric molecule containing the human β-2 microglobulin and HLA-A2.1 α1 and α2 domains with a mouse 3 and transmembrane domain (AAD HLA-A2). Mice entered the study at approximately 6-8 weeks of age. Male mice weighed between 18 and 23 g, female mice weighed between 16-21 g. Mice were housed in groups of 3 to 5 mice per cage. All animal work has been conducted in accordance with the Guide for Care and Use of Laboratory Animals of the National Institute of Health with approval from the Saint Louis University Institutional Animal Care and Use Committee.

### Virus

The ACAM2000 (Acambis, Inc.) smallpox vaccine is a live virus derived from the original Dryvax (Wyeth Laboratories, Inc.). MPXV is a member of the Orthopoxvirus family and is 95% genetically identical to the smallpox vaccine. Vaccination with the smallpox vaccine confers protection against MPXV infection (Handley et al., 2009). MPXV Zaire 79 was obtained from the Saint Louis University School of Medicine, department of molecular microbiology and immunology Biosafety Level 3/Select Agent program. ACAM2000 smallpox vaccine was a gift from the center for disease control (Atlanta, Ga.). Both MPXV and ACAM2000 smallpox vaccine titers and infectivity were estimated plaque forming assay (Handley et al., 2009; Parker et al., 2014). 1×10⁵ BSC-1 cells were plated in DMEM+ 10% FCS in 24-well plates at 0.5 mL final volume and cultured for 24 hours at 37° C. and 5% CO₂. Stock viral suspensions were serially diluted 1:10 in DMEM+1% FCS, and 300 μL of supernatant from BSC-1 cells was removed. 100 μL of diluted virus solutions were added to BSC-1 cells in triplicate, gently swirled, and incubated for 1 hour at 37° C. and 5% CO₂. Overlay media (1% carboxyl methyl cellulose in DMEM with 5% FCS) was warmed to 37° C. and 1 mL added to each well and cultures were returned to the incubator. Cultures were maintained for 3-4 days at 37° C. until plaques were visible. Plaques were visualized and virus inactivated with 200 μL 0.3% crystal violet/10% formalin solution for 1 hour. All liquid was aspirated and plates were washed with water, inverted, and dried. Plaques were counted for each viral dilution, the average plaque count is divided by product of the dilution and volume of virus overlay.

### Primary T Cell Cultures

Spleens from male and female mice previously vaccinated with the ACAM2000 smallpox vaccine were mechanically disrupted to form single cell suspensions. Cells were filtered through a 40 um nylon mesh cell strainer and washed with complete RPMI supplemented with 10% fetal bovine serum (cRPMI10F, 1% Penicillin/Streptomycin (Sigma-Aldrich, P0871, 10,000 U/10 mg per mL), 1% L-glutamine (Sigma-Aldrich, G7513, 200 mM), 1% non-essential amino acids (Sigma-Aldrich, M7145, 100×), 1% HEPES (Sigma-Aldrich, H3537), 1% sodium pyruvate (Sigma-Aldrich, S8636, 100 mM)). Red blood cells were lysed by incubation with 5 mL 1×ACK (ammonium-chloride-potassium) lysing buffer for 5 minutes at 37° C. and 5% CO₂. Cells were washed with cRPMI10F and cell count determined. Splenocytes were cultured in cRPMI10F at a concentration of 1×10⁶/mL at 37° C. and 5% CO₂.

### Vaccination/Infection of HLA-A2 Mice

At 6-8 weeks of age mice were anesthetized by intraperitoneal injection with 0.01 mL/g body weight Ketamine (6 mg/mL)/Xylazine (0.5 mg/mL) cocktail and intranasally administered sub-lethal doses of either the ACAM2000 smallpox vaccine (approximately 5×10⁴ PFU) or MPXV Zaire-79 strain (0.5×10⁴ PFU) in 25 μL total volume (12.5 μL per naris) (Moutaftsi et al., 2009, Parker and Buller, 2013, Parker et al., 2014, Stabenow et al., 2010). Male and female mice were entered into each treatment group with approximate equality by cage. All mice recovered from the viral challenge. Blood samples were collected via the submandibular cheek bleed 1 week prior to vaccination or infection and at 2 weeks, 8 weeks, 16 weeks, and 9 months post-vaccination or post-infection.

### Anti-Pox Serum ELISA

Serum from HLA-A2 mice was collected by aspiration from whole blood prior to vaccination or infection and at 2-weeks and 8-weeks after viral exposure by centrifugation at 11,000 g for 5 min. Samples were tested for vaccinia-specific serum antibody by neutralizing antibody ELISA (Frey et al., 2002). 96-well maxSorp plates (ThermoFisher 44-2404-21) were coated with crude extract from lysed BSC-1 cells infected with live ACAM2000 smallpox vaccine. Plates were washed 3× with PBS and wells were coated with approximately 5×10⁴ PFU live ACAM2000 diluted in carbonate coating buffer (0.1M Na₂CO₃ 0.1M NaHCO₃ pH 9.3) in 100 ul overnight at 4° C. Wells were washed and blocked with blocking buffer containing 5% BSA in PBS for 30 min at room temperature (RT). Plates were washed 3× with PBS and serum samples diluted 1:20 in blocking buffer were added (in duplicate) to wells and incubated for 2 hours at room temperature (RT). Plate was washed 3× with PBS, then 100 μL anti-mouse HRP-conjugated antibody (Sigma A8924) was diluted 1:2500 in blocking buffer was added and allowed to incubate for 1 h. Plates were washed 3× with PBS and 75 μl of True Blue peroxidase substrate (KPL, 71-00-65) was added to each well and incubated for 15 min in the dark at room temperature (RT). 75 μl of 1N HCl was added to each well and OD measured at 450 nm.

### Sample Preparation and DNA Sequencing

Genomic DNA was extracted and purified using the QIAGEN Blood and Tissue Kit (item #69506). Genomic DNA was amplified using multiplexed primers targeting all V and J gene segments as described previously (Carlson et al., 2013). tcrb CDR3 regions were amplified and sequenced using ImmunoSEQ (Adaptive Biotechnologies). Synthetic templates mimicking natural V(D)J rearrangements were used to measure and correct potential amplification bias (Carlson et al., 2013, Wolf et al., 2016). CDR3 segments were annotated according to the International ImMunoGeneTics (IMGT) collaboration (Yousfi Monod et al., 2004), identifying V, D, and J genes contributing to each rearrangement.

### T-cell Expansion Assay

Cells were maintained in cRPMI10F media alone or supplemented with 0.2MOI live ACAM2000 smallpox vaccine and allowed to incubate at 37° C. for 5 days. T cell blasting and proliferation were observed prior to DNA extraction for Immunosequencing. Cultured cells were pelleted by centrifugation at 1500 rpm for 10 minutes and supernatant aspirated. Pelleted cells were re-suspended in 200 μl PBS prior to DNA extraction.

### Flow Cytometry and HLA-A2 Tetramer Sorting

PE-conjugated HLA-A2.1 chimeric tetramers (HLA-A2 tetramers) loaded with vaccinia-derived peptides (Gilchuk et al., 2013) were a kind gift from Dr. Sebastian Joyce (Vanderbilt). Pooled splenocytes from previously vaccinated mice were stained with T cell and B cell lineage markers CD4 (ebioscience, PerCP-efluor710, clone GK1.5), CD8 (BD Horizon, BV450, clone 53-6.7), and CD19 (BD Horizon, BV605, clone D3) for 20 min at 4° C. Cells were washed 2× in PBS+2% FBS, then cells were incubated with vaccinia-peptide loaded HLA-A2 tetramers (25 μg/mL, 2 μL per 1×106 splenocytes) for 1 h at RT. TCR-epitope-tetramer binding CD4-CD19-CD8+ T cell populations were purified by FACS into tetramer− and TCR-epitope-tetramer binding tetramer+ populations. Tetramer sorted T cells were centrifuged at 1500 rpm for 10 minutes. Supernatant was aspirated and cells re-suspended in 200 μl PBS prior to DNA extraction.

### VATS and MATS Library Development

Alignment of shared and non-shared TCRO sequences was completed using ImmunoSEQ software provided by Adaptive Biotechnologies. Alignments of all 2-week and 8-week post-ACAM2000 smallpox vaccination TCR repertoires were used to identify public TCR sequences. The list of public TCR sequences was compared to alignments of all TCRO repertoires from naive samples in order to perform an association analysis to identify a set of TCRβ sequences that had significantly increased incidence among vaccinated but not naive TCRβ repertoires. For the association analysis, we performed a one-tailed Fisher's Exact test on all sequences, comparing the number of naive and vaccinated samples each TCRβ sequence was present in.

To determine an optimal p value threshold for identifying VATS, we applied a heuristic test that selected the optimal p value threshold based on the “coverage” provided by the library for both vaccinated (Cᵥ) and naive samples (Cn). “Coverage” is defined as the summation of the number of samples containing each VATS divided by the number of samples. In the equations below, xᵢ denotes the number of vaccinated samples a single TCRβ is identified in (yᵢ denotes naive samples) and nᵥ represents the number of samples in the training data (nₙ represents naive samples).

$${C}_{v} = \frac{{\sum }_{i = 1}^{I} {x}_{i}}{{n}_{v}} {C}_{n} = \frac{{\sum }_{i = 1}^{I} {y}_{i}}{{n}_{n}}$$

The ratio of Cᵥ to Cₙ is determined for each p value. Additionally, the Cᵥ and Cₙ of each p value (rounded to the nearest whole integer), are applied to a one-tailed Fisher's Exact test against the total number of sequences in the prospective library to determine if there is sufficient coverage to distinguish vaccinated from naive samples (p<0.05). The p value with the largest Cᵥ:Cₙ ratio and offers significant coverage to distinguish vaccinated from naive samples was chosen.

### Classification of Vaccinated or MPXV Exposed Versus Naïve Samples

To distinguish between vaccinated and naive samples, the proportion of VATS present in a sample was compared against the normal distribution of the naive and vaccinated training data. Normal distribution for our purposes is used to measure the distance a sample is from the mean. The normal distributions for the naive and vaccinated populations in our training data were calculated based on a function of the difference between a single sample value (x) and the mean of a set of data (μ) over the standard deviation of that set of data (σ). The greater the value, the greater association that sample has with the training group. By comparing a sample against the normal distribution of vaccinated and naive training groups, we can determine which group a sample is more statistically associated with.

$$f ( x \vert μ , {σ}^{2} ) = ( \frac{1}{\sqrt{2 π {σ}^{2}}} ) {e}^{- \frac{{( x - μ )}^{2}}{2 {σ}^{2}}}$$

The Leave-one-out (LOO) analysis was completed as previously described (Emerson and DeWitt, 2017). Briefly, all samples associated with a single mouse were removed from the training data and the VATS library was re-derived using the remaining training cohort. The % VATS was calculated for all samples and used to train the diagnostic classifier.

### Statistical Analysis

Alignment of shared and non-shared TCRs was completed using ImmunoSeq software provided by Adaptive Biotechnologies. Graphical analyses were created using GraphPad Prism 5.0. 1-way ANOVA and Bonferroni's multiple comparison test was accomplished using GraphPad Prism 5.0. Pearson correlation was calculated using GraphPad Prism 5.0.

### Example 1: Vaccination and Infection of AAD Mice with Orthopoxvirus

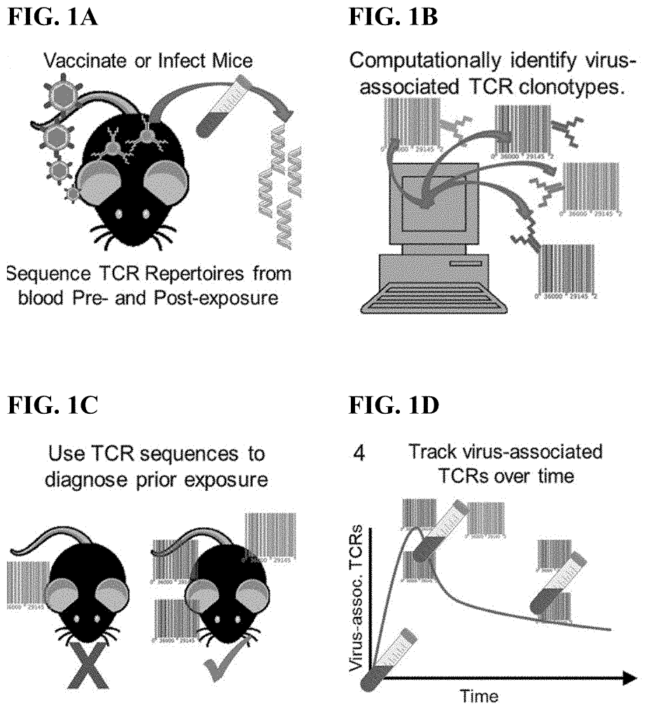

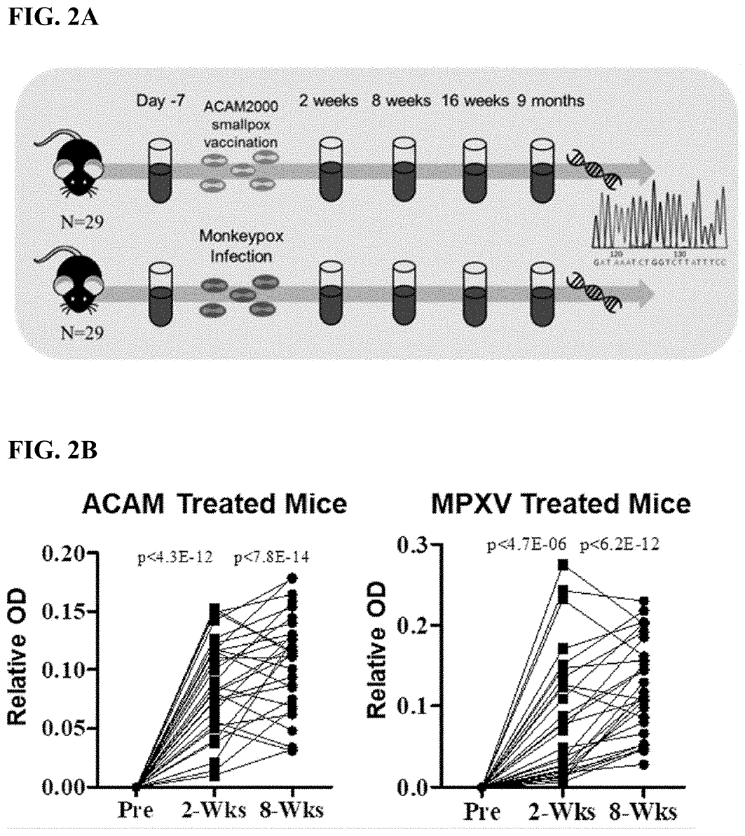

An extensive TCR sequence database was generated from a large cohort of HLA-A2 transgenic (AAD) mice before and up to 9 months after administration of the ACAM2000 smallpox vaccine or infection with MPXV (FIG. 1A). Fifty-eight AAD HLA-A2 transgenic mice (HLA-A2), pooled from three independent experiments, were entered into the study. The expression of the HLA-A2 transgenic molecule allows a portion of the virus-specific T cell response to be generated in the context of human MHC molecules (Kotturi et al., 2009). A cohort of 29 HLA-A2 mice were vaccinated with the smallpox vaccine, and another cohort of 29 mice were infected with MPXV, and blood samples were collected over time (FIG. 2A). Increased pox-specific antibodies were observed in serum 2 and 8 weeks post-vaccination or infection compared with naive in all mice by ELISA, confirming the generation of an immune response against the vaccine or infection (FIG. 2B). Blood samples (approximately 100 μL) collected before (naive) and 2 weeks, 8 weeks, 16 weeks, and 9 months after exposure had genomic DNA purified for immunosequencing of the TCRβ repertoire to identify all TCRβ clonotypes present in each sample. A unique TCRβ clonotype in this study is defined as a unique combination of a V gene, CDR3 amino acid sequence, and J gene. From the genomic DNA purified from the whole blood, 2.85×106 unique TCRβ clonotypes were identified from the TCR repertoires of all naive (n=32), vaccinated (n=99), and infected (n=114) samples (Table 5). In brief, after confirming the generation of a pox-specific immune response in all mice, high-throughput sequencing data of TCRβ genes from a large cohort of mice were used to compile a large database of TCR clonotypes in order to computationally identify vaccine- and infection-specific TCRs.

TABLE 5
TCRβ Sequence rearrangements and
Clonotypes in Vaccinated and Infected Mice

| | | | | Number of | Total number |
|---|---|---|---|---|---|
| | | | Number of | unique | of |
| Treatment | Time Point | | samples (n) | clonotypes | rearrangements |
| Naïve samples | | | 32 | 700,000 | 1,698,000 |
| ACAM | 2 | weeks | 29 | 391,500 | 700,000 |
| vaccination | 8 | weeks | 29 | 185,000 | 420,000 |
| | 16 | weeks | 18 | 222,500 | 397,500 |
| | 9 | months | 23 | 209,000 | 316,000 |
| MPXV infected | 2 | weeks | 29 | 300,000 | 538,500 |
| | 8 | weeks | 29 | 177,500 | 326,000 |
| | 16 | weeks | 29 | 374,500 | 705,000 |
| | 9 | months | 27 | 285,000 | 433,000 |

Consolidated data referencing the total number of mice, unique TCRβ sequences (clonotypes), total number of rearranged TCRβ genes sequenced for each time point for naive, ACAM2000 vaccinated, and MPXV infected samples

### Example 2: Development of the VATS Library

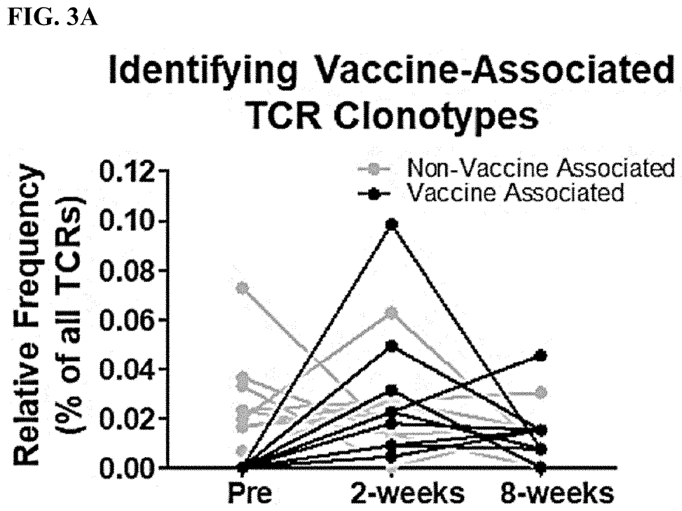

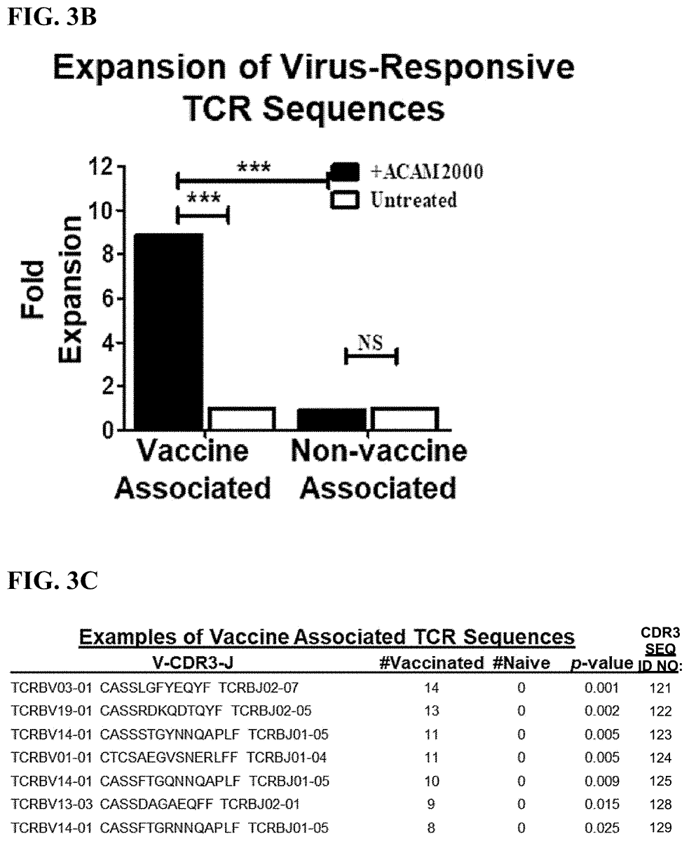

TCR repertoires from whole blood of mice pre- and post-vaccination were analyzed to computationally identify TCR clonotypes present post-vaccination but absent pre-vaccination versus sequences present pre- and post-vaccination (FIG. 1). Within individual mice, TCR clonotypes were identified that were expanded in the blood post-vaccination (2 and 8 weeks) and absent prior to vaccination (vaccine-associated) in addition to TCR clonotypes present pre- and post-vaccination (non-vaccine-associated) (FIG. 3A).

Next it was determined whether the computationally identified VATS contained TCR clonotypes that functionally expanded in response to smallpox vaccine. Splenocytes of mice from the original cohort 12 weeks after vaccination were cultured with or without the smallpox vaccine for 5 days to induce expansion of vaccine-specific T cells in vitro. Intra-mouse analysis of the TCR repertoires pre- and 2 and 8 weeks post-vaccination were compared with the libraries from splenocytes cultured with or without ACAM2000. It has been previously shown that the smallpox vaccine does not induce bystander activation of CD8+ T cells, which leads us to conclude that TCR sequences from proliferating T cells in this experimental design are virus specific (Miller et al., 2008). The relative abundances of vaccine-associated and non-vaccine-associated TCR clonotypes were compared between vaccine-stimulated and unstimulated cultures. Post-vaccine-associated sequences were significantly expanded (8.9-fold, p<0.0001) in the vaccine-stimulated versus unstimulated controls; this is significantly greater (p<0.0001) than the expansion (0.94-fold) measured in non-VATS (FIG. 3B). These data show that computational identification of vaccine-associated TCRβ clonotypes enriches for virus-specific TCR sequences.

To distinguish between TCR repertoires from naive and exposed samples, a vaccine-associated public TCRβ library was generated. Pre- and post-vaccination TCRβ sequence libraries were analyzed, computationally detecting the virus-specific T cell response by identifying sequences that were statistically associated with post-vaccination samples. TCRβ sequences from all naive (n=32) and 2 and 8 week post-vaccination TCRβ repertoires (n=58) were used for this analysis. Each TCRβ clonotype identified from the 58 post-vaccinated samples (approximately 576,000) was analyzed using a one-tailed Fisher's exact test for association with vaccinated libraries compared with naive libraries (FIG. 3C). Vaccine-associated TCRβ libraries were designed at various p values to determine a threshold to appropriately filter the virus-associated TCRβ sequences for use in generating the diagnostic assay. Using a heuristic test, comparing the coverage (average number of vaccine-associated sequences present in a single sample) between vaccinated and naive mice, a significance threshold of 0.11 (by one-tailed Fisher's exact test) was identified as the optimal exclusionary threshold (see STAR Methods). Using this threshold, 315 individual VATS were identified (Table 6).

TABLE 6
TCRβ alleles associated with small pox vaccination in mice

| | | | | CDR3 |
|---|---|---|---|---|
| | # Vaccinated | # Naïve | | SEQ ID |
| V-CDR3-J | Samples | Samples | p-value | NO: |
| TCRBV03-01 CASSLGFYEQYF TCRBJ02-07 | 14/58 | 0/32 | 0.0011 | 121 |
| TCRBV19-01 CASSRDKQDTQYF TCRBJ02-05 | 13/58 | 0/32 | 0.0019 | 122 |
| TCRBV14-01 CASSSTGYNNQAPLF TCRBJ01-05 | 11/58 | 0/32 | 0.0055 | 123 |
| TCRBV01-01 CTCSAEGVSNERLFF TCRBJ01-04 | 11/58 | 0/32 | 0.0055 | 124 |
| TCRBV14-01 CASSFTGQNNQAPLF TCRBJ01-05 | 10/58 | 0/32 | 0.0091 | 125 |
| TCRBV13-01 CASSRQGGDERLFF TCRBJ01-04 | 10/58 | 0/32 | 0.0091 | 126 |
| TCRBV29-01 CASGNTEVFF TCRBJ01-01 | 10/58 | 0/32 | 0.0091 | 127 |
| TCRBV13-03 CASSDAGAEQFF TCRBJ02-01 | 9/58 | 0/32 | 0.0151 | 128 |
| TCRBV14-01 CASSFTGRNNQAPLF TCRBJ01-05 | 8/58 | 0/32 | 0.0247 | 129 |
| TCRBV19-01 CASSRDRYAEQFF TCRBJ02-01 | 8/58 | 0/32 | 0.0247 | 130 |
| TCRBV01-01 CTCSADLGTSAETLYF TCRBJ02-03 | 8/58 | 0/32 | 0.0247 | 131 |
| TCRBV12-02 CASSPTTSAETLYF TCRBJ02-03 | 7/58 | 0/32 | 0.0402 | 132 |
| TCRBV04-01 CASSHRDGQDTQYF TCRBJ02-05 | 7/58 | 0/32 | 0.0402 | 133 |
| TCRBV13-02 CASGEGLGEQYF TCRBJ02-07 | 7/58 | 0/32 | 0.0402 | 134 |
| TCRBV05-01 CASSQDRQGYEQYF TCRBJ02-07 | 7/58 | 0/32 | 0.0402 | 135 |
| TCRBV03-01 CASSSDRHQDTQYF TCRBJ02-05 | 7/58 | 0/32 | 0.0402 | 136 |
| TCRBV05-01 CASSQDLGPYEQYF TCRBJ02-07 | 7/58 | 0/32 | 0.0402 | 137 |
| TCRBV19-01 CASSIRAEQYF TCRBJ02-07 | 7/58 | 0/32 | 0.0402 | 138 |
| TCRBV12-02 CASSLTGGSSYEQYF TCRBJ02-07 | 7/58 | 0/32 | 0.0402 | 139 |
| TCRBV01-01 CTCSAAGTGVGNTLYF TCRBJ01-03 | 7/58 | 0/32 | 0.0402 | 140 |
| TCRBV04-01 CASSLTAYEQYF TCRBJ02-07 | 7/58 | 0/32 | 0.0402 | 141 |
| TCRBV05-01 CASSQEGLGGREQYF TCRBJ02-07 | 7/58 | 0/32 | 0.0402 | 142 |
| TCRBV13-03 CASSDPGGNERLFF TCRBJ01-04 | 7/58 | 0/32 | 0.0402 | 143 |
| TCRBV05-01 CASSQEGINQDTQYF TCRBJ02-05 | 7/58 | 0/32 | 0.0402 | 144 |
| TCRBV12-01 CASSLGTVSYNSPLYF TCRBJ01-06 | 7/58 | 0/32 | 0.0402 | 145 |
| TCRBV05-01 CASSQETGNTEVFF TCRBJ01-01 | 7/58 | 0/32 | 0.0402 | 146 |
| TCRBV31-01 CAWSLAGDNQAPLF TCRBJ01-05 | 7/58 | 0/32 | 0.0402 | 147 |
| TCRBV05-01 CASSQEGTGTETLYF TCRBJ02-03 | 7/58 | 0/32 | 0.0402 | 148 |
| TCRBV14-01 CASSSTGRNNQAPLF TCRBJ01-05 | 6/58 | 0/32 | 0.0650 | 149 |
| TCRBV13-02 CASGDWGGATGQLYF TCRBJ02-02 | 6/58 | 0/32 | 0.0650 | 150 |
| TCRBV13-02 CASGDAAGGTGQLYF TCRBJ02-02 | 6/58 | 0/32 | 0.0650 | 151 |
| TCRBV19-01 CASSPTTYEQYF TCRBJ02-07 | 6/58 | 0/32 | 0.0650 | 152 |
| TCRBV03-01 CASSLSGGYEQYF TCRBJ02-07 | 6/58 | 0/32 | 0.0650 | 153 |
| TCRBV13-03 CASSPDSYEQYF TCRBJ02-07 | 6/58 | 0/32 | 0.0650 | 154 |
| TCRBV05-01 CASSPGTNNQAPLF TCRBJ01-05 | 6/58 | 0/32 | 0.0650 | 155 |
| TCRBV13-03 CASSPQGAGNTLYF TCRBJ01-03 | 6/58 | 0/32 | 0.0650 | 156 |
| TCRBV04-01 CASSWTGSGNTLYF TCRBJ01-03 | 6/58 | 0/32 | 0.0650 | 157 |
| TCRBV13-01 CASRLRDWGYEQYF TCRBJ02-07 | 6/58 | 0/32 | 0.0650 | 158 |
| TCRBV02-01 CASSQDPGGGYEQYF TCRBJ02-07 | 6/58 | 0/32 | 0.0650 | 159 |
| TCRBV19-01 CASSTGGVYEQYF TCRBJ02-07 | 6/58 | 0/32 | 0.0650 | 160 |
| TCRBV29-01 CASSTSNSDYTF TCRBJ01-02 | 6/58 | 0/32 | 0.0650 | 161 |
| TCRBV01-01 CTCSARDTYEQYF TCRBJ02-07 | 6/58 | 0/32 | 0.0650 | 162 |
| TCRBV13-02 CASGGTGVYEQYF TCRBJ02-07 | 6/58 | 0/32 | 0.0650 | 163 |
| TCRBV13-02 CASGTGGSYEQYF TCRBJ02-07 | 6/58 | 0/32 | 0.0650 | 164 |
| TCRBV13-01 CASSDAIYEQYF TCRBJ02-07 | 6/58 | 0/32 | 0.0650 | 165 |
| TCRBV03-01 CASSLAPDSGNTLYF TCRBJ01-03 | 6/58 | 0/32 | 0.0650 | 166 |
| TCRBV04-01 CASSLRDGQDTQYF TCRBJ02-05 | 6/58 | 0/32 | 0.0650 | 167 |
| TCRBV03-01 CASSSGDSDYTF TCRBJ01-02 | 6/58 | 0/32 | 0.0650 | 168 |
| TCRBV01-01 CTCSARLGGYAEQFF TCRBJ02-01 | 6/58 | 0/32 | 0.0650 | 169 |
| TCRBV12-01 CASSPPGQLYF TCRBJ02-02 | 6/58 | 0/32 | 0.0650 | 170 |
| TCRBV01-01 CTCSAGGGAGEQYF TCRBJ02-07 | 6/58 | 0/32 | 0.0650 | 171 |
| TCRBV13-01 CASRRQGNSDYTF TCRBJ01-02 | 6/58 | 0/32 | 0.0650 | 172 |
| TCRBV13-01 CASSDGTEQYF TCRBJ02-07 | 6/58 | 0/32 | 0.0650 | 173 |
| TCRBV13-03 CASSDQGSNERLFF TCRBJ01-04 | 6/58 | 0/32 | 0.0650 | 174 |
| TCRBV16-01 CASSPTGGGNTLYF TCRBJ01-03 | 6/58 | 0/32 | 0.0650 | 175 |
| TCRBV19-01 CASSRDNNYAEQFF TCRBJ02-01 | 6/58 | 0/32 | 0.0650 | 176 |
| TCRBV31-01 CAWSRNSDYTF TCRBJ01-02 | 6/58 | 0/32 | 0.0650 | 177 |
| TCRBV29-01 CASSFQQDTQYF TCRBJ02-05 | 6/58 | 0/32 | 0.0650 | 178 |
| TCRBV15-01 CASSGDNAETLYF TCRBJ02-03 | 6/58 | 0/32 | 0.0650 | 179 |
| TCRBV26-01 CASSLGLNQDTQYF TCRBJ02-05 | 6/58 | 0/32 | 0.0650 | 180 |
| TCRBV13-02 CASGPGRISNERLFF TCRBJ01-04 | 6/58 | 0/32 | 0.0650 | 181 |
| TCRBV13-03 CASSGTVNYAEQFF TCRBJ02-01 | 6/58 | 0/32 | 0.0650 | 182 |
| TCRBV03-01 CASSLNSNSDYTF TCRBJ01-02 | 6/58 | 0/32 | 0.0650 | 183 |
| TCRBV03-01 CASSPDSSAETLYF TCRBJ02-03 | 6/58 | 0/32 | 0.0650 | 184 |
| TCRBV26-01 CASSPGQTEVFF TCRBJ01-01 | 6/58 | 0/32 | 0.0650 | 185 |
| TCRBV29-01 CASSPTGSGNTLYF TCRBJ01-03 | 6/58 | 0/32 | 0.0650 | 186 |
| TCRBV02-01 CASSQDGGGTGQLYF TCRBJ02-02 | 6/58 | 0/32 | 0.0650 | 187 |
| TCRBV05-01 CASSQGYQDTQYF TCRBJ02-05 | 6/58 | 0/32 | 0.0650 | 188 |
| TCRBV16-01 CASSFKDTQYF TCRBJ02-05 | 6/58 | 0/32 | 0.0650 | 189 |
| TCRBV19-01 CASSIAGTGNERLFF TCRBJ01-04 | 6/58 | 0/32 | 0.0650 | 190 |
| TCRBV12-01 CASSPDRGQNTLYF TCRBJ02-04 | 6/58 | 0/32 | 0.0650 | 191 |
| TCRBV03-01 CASSWTGQDTQYF TCRBJ02-05 | 6/58 | 0/32 | 0.0650 | 192 |
| TCRBV04-01 CASSYREDTQYF TCRBJ02-05 | 6/58 | 0/32 | 0.0650 | 193 |
| TCRBV13-03 CASTGQANTEVFF TCRBJ01-01 | 6/58 | 0/32 | 0.0650 | 194 |
| TCRBV01-01 CTCSADINQDTQYF TCRBJ02-05 | 6/58 | 0/32 | 0.0650 | 195 |
| TCRBV13-02 CASGETGGNTEVFF TCRBJ01-01 | 6/58 | 0/32 | 0.0650 | 196 |
| TCRBV13-02 CASGPGQSNTEVFF TCRBJ01-01 | 6/58 | 0/32 | 0.0650 | 197 |
| TCRBV13-01 CASSGDNSAETLYF TCRBJ02-03 | 6/58 | 0/32 | 0.0650 | 198 |
| TCRBV12-02 CASSLEAGGAETLYF TCRBJ02-03 | 6/58 | 0/32 | 0.0650 | 199 |
| TCRBV12-01 CASSLQNTLYF TCRBJ02-04 | 6/58 | 0/32 | 0.0650 | 200 |
| TCRBV26-01 CASSLRGEVFF TCRBJ01-01 | 6/58 | 0/32 | 0.0650 | 201 |
| TCRBV03-01 CASSPGQGDTEVFF TCRBJ01-01 | 6/58 | 0/32 | 0.0650 | 202 |
| TCRBV01-01 CTCSAGTGHTEVFF TCRBJ01-01 | 6/58 | 0/32 | 0.0650 | 203 |
| TCRBV03-01 CASSPRTGGSAETLYF TCRBJ02-03 | 14/58 | 1/32 | 0.0077 | 204 |
| TCRBV16-01 CASSLGTGVNQAPLF TCRBJ01-05 | 12/58 | 1/32 | 0.0193 | 205 |
| TCRBV01-01 CTCSAGTKDTQYF TCRBJ02-05 | 10/58 | 1/32 | 0.0456 | 206 |
| TCRBV04-01 CASSPTSYEQYF TCRBJ02-07 | 9/58 | 1/32 | 0.0687 | 207 |
| TCRBV03-01 CASSLVGASAETLYF TCRBJ02-03 | 9/58 | 1/32 | 0.0687 | 208 |
| TCRBV20-01 CGAREGEDTQYF TCRBJ02-05 | 9/58 | 1/32 | 0.0687 | 209 |
| TCRBV02-01 CASSQDRDKYEQYF TCRBJ02-07 | 8/58 | 1/32 | 0.1019 | 210 |
| TCRBV15-01 CASSRQGGDERLFF TCRBJ01-04 | 8/58 | 1/32 | 0.1019 | 211 |
| TCRBV16-01 CASSLGGPYEQYF TCRBJ02-07 | 8/58 | 1/32 | 0.1019 | 212 |
| TCRBV13-03 CASRNTGQLYF TCRBJ02-02 | 8/58 | 1/32 | 0.1019 | 213 |
| TCRBV16-01 CASSRQGNYAEQFF TCRBJ02-01 | 8/58 | 1/32 | 0.1019 | 214 |
| TCRBV29-01 CASSLGGANTGQLYF TCRBJ02-02 | 8/58 | 1/32 | 0.1019 | 215 |
| TCRBV13-02 CASGDAGGRNTLYF TCRBJ02-04 | 8/58 | 1/32 | 0.1019 | 216 |
| TCRBV13-02 CASGGGLQDTQYF TCRBJ02-05 | 8/58 | 1/32 | 0.1019 | 217 |
| TCRBV03-01 CASSFDWGQDTQYF TCRBJ02-05 | 8/58 | 1/32 | 0.1019 | 218 |
| TCRBV03-01 CASSLGLGVNQDTQYF TCRBJ02-05 | 8/58 | 1/32 | 0.1019 | 219 |
| TCRBV12-02 CASSLGQSQNTLYF TCRBJ02-04 | 8/58 | 1/32 | 0.1019 | 220 |
| TCRBV29-01 CASSLSGNQDTQYF TCRBJ02-05 | 8/58 | 1/32 | 0.1019 | 221 |
| TCRBV03-01 CASSSGLQDTQYF TCRBJ02-05 | 8/58 | 1/32 | 0.1019 | 222 |
| TCRBV31-01 CAWSPDRANTEVFF TCRBJ01-01 | 8/58 | 1/32 | 0.1019 | 223 |
| TCRBV15-01 CASSLAGGNTEVFF TCRBJ01-01 | 8/58 | 1/32 | 0.1019 | 224 |
| TCRBV16-01 CASSPGLGEDTQYF TCRBJ02-05 | 8/58 | 1/32 | 0.1019 | 225 |
| TCRBV05-01 CASSQDGGASQNTLYF TCRBJ02-04 | 8/58 | 1/32 | 0.1019 | 226 |
| TCRBV31-01 CAWSLDQDTQYF TCRBJ02-05 | 8/58 | 1/32 | 0.1019 | 227 |
| TCRBV13-01 CASSEGSQDTQYF TCRBJ02-05 | 13/58 | 2/32 | 0.0419 | 228 |
| TCRBV19-01 CASSSGTANTEVFF TCRBJ01-01 | 13/58 | 2/32 | 0.0419 | 229 |
| TCRBV13-02 CASGDVGQGNERLFF TCRBJ01-04 | 12/58 | 2/32 | 0.0612 | 230 |
| TCRBV29-01 CASSLPGTNERLFF TCRBJ01-04 | 11/58 | 2/32 | 0.0880 | 231 |
| TCRBV26-01 CASSLSGNTGQLYF TCRBJ02-02 | 11/58 | 2/32 | 0.0880 | 232 |
| TCRBV01-01 CTCSAGQNNQAPLF TCRBJ01-05 | 11/58 | 2/32 | 0.0880 | 233 |
| TCRBV16-01 CASSLGGAREQYF TCRBJ02-07 | 11/58 | 2/32 | 0.0880 | 234 |
| TCRBV13-03 CASSDLGGQDTQYF TCRBJ02-05 | 11/58 | 2/32 | 0.0880 | 235 |
| TCRBV02-01 CASSQESQNTLYF TCRBJ02-04 | 11/58 | 2/32 | 0.0880 | 236 |
| TCRBV13-01 CASSGTGGYAEQFF TCRBJ02-01 | 15/58 | 3/32 | 0.0512 | 237 |
| TCRBV02-01 CASSQDNSQNTLYF TCRBJ02-04 | 13/58 | 3/32 | 0.1012 | 238 |
| TCRBV12-01 CASSLGGAGNTLYF TCRBJ01-03 | 15/58 | 4/32 | 0.1100 | 239 |
| TCRBV02-01 CASSQEGWGNQDTQYF TCRBJ02-05 | 20/58 | 6/32 | 0.0895 | 240 |
| TCRBV02-01 CASSQDLWGSSQNTLYF TCRBJ02-04 | 5/58 | 0/32 | 0.1043 | 241 |
| TCRBV04-01 CASSPTGEEQYF TCRBJ02-07 | 5/58 | 0/32 | 0.1043 | 242 |
| TCRBV01-01 CTCSVTDSGNTLYF TCRBJ01-03 | 5/58 | 0/32 | 0.1043 | 243 |
| TCRBV15-01 CASSLDNAETLYF TCRBJ02-03 | 5/58 | 0/32 | 0.1043 | 244 |
| TCRBV01-01 CTCSAEGGRGEQYF TCRBJ02-07 | 5/58 | 0/32 | 0.1043 | 245 |
| TCRBV13-03 CASSDWGEGEQYF TCRBJ02-07 | 5/58 | 0/32 | 0.1043 | 246 |
| TCRBV13-03 CASSEDSGNTLYF TCRBJ01-03 | 5/58 | 0/32 | 0.1043 | 247 |
| TCRBV13-01 CASSRGNSDYTF TCRBJ01-02 | 5/58 | 0/32 | 0.1043 | 248 |
| TCRBV03-01 CASSSRDRGDSDYTF TCRBJ01-02 | 5/58 | 0/32 | 0.1043 | 249 |
| TCRBV13-02 CASGGRYEQYF TCRBJ02-07 | 5/58 | 0/32 | 0.1043 | 250 |
| TCRBV13-01 CASSDSGREQYF TCRBJ02-07 | 5/58 | 0/32 | 0.1043 | 251 |
| TCRBV03-01 CASSLLGEQYF TCRBJ02-07 | 5/58 | 0/32 | 0.1043 | 252 |
| TCRBV14-01 CASSRSYEQYF TCRBJ02-07 | 5/58 | 0/32 | 0.1043 | 253 |
| TCRBV31-01 CAWSPRGNSDYTF TCRBJ01-02 | 5/58 | 0/32 | 0.1043 | 254 |
| TCRBV01-01 CTCSADRGDYAEQFF TCRBJ02-01 | 5/58 | 0/32 | 0.1043 | 255 |
| TCRBV01-01 CTCSAGTGGSNERLFF TCRBJ01-04 | 5/58 | 0/32 | 0.1043 | 256 |
| TCRBV13-02 CASGDQGAGERLFF TCRBJ01-04 | 5/58 | 0/32 | 0.1043 | 257 |
| TCRBV13-02 CASGDTGAGNTLYF TCRBJ01-03 | 5/58 | 0/32 | 0.1043 | 258 |
| TCRBV13-02 CASGEGAYEQYF TCRBJ02-07 | 5/58 | 0/32 | 0.1043 | 259 |
| TCRBV03-01 CASSATGGEQYF TCRBJ02-07 | 5/58 | 0/32 | 0.1043 | 260 |
| TCRBV15-01 CASSDNYAEQFF TCRBJ02-01 | 5/58 | 0/32 | 0.1043 | 261 |
| TCRBV29-01 CASSFGGANSDYTF TCRBJ01-02 | 5/58 | 0/32 | 0.1043 | 262 |
| TCRBV12-01 CASSLKGSGNTLYF TCRBJ01-03 | 5/58 | 0/32 | 0.1043 | 263 |
| TCRBV26-01 CASSLSLSNERLFF TCRBJ01-04 | 5/58 | 0/32 | 0.1043 | 264 |
| TCRBV19-01 CASSPGQGAYEQYF TCRBJ02-07 | 5/58 | 0/32 | 0.1043 | 265 |
| TCRBV04-01 CASSPLGGPYEQYF TCRBJ02-07 | 5/58 | 0/32 | 0.1043 | 266 |
| TCRBV02-01 CASSQDWGLSYEQYF TCRBJ02-07 | 5/58 | 0/32 | 0.1043 | 267 |
| TCRBV02-01 CASSQEGGGAYEQYF TCRBJ02-07 | 5/58 | 0/32 | 0.1043 | 268 |
| TCRBV04-01 CASSRDSGNTLYF TCRBJ01-03 | 5/58 | 0/32 | 0.1043 | 269 |
| TCRBV19-01 CASSRTGVYEQYF TCRBJ02-07 | 5/58 | 0/32 | 0.1043 | 270 |
| TCRBV13-01 CASSDPGGTETLYF TCRBJ02-03 | 5/58 | 0/32 | 0.1043 | 271 |
| TCRBV13-01 CASSDQGAYAEQFF TCRBJ02-01 | 5/58 | 0/32 | 0.1043 | 272 |
| TCRBV13-01 CASSDRDTGQLYF TCRBJ02-02 | 5/58 | 0/32 | 0.1043 | 273 |
| TCRBV14-01 CASSFTGDEQYF TCRBJ02-07 | 5/58 | 0/32 | 0.1043 | 274 |
| TCRBV19-01 CASSMSYEQYF TCRBJ02-07 | 5/58 | 0/32 | 0.1043 | 275 |
| TCRBV12-01 CASSPGDSGNTLYF TCRBJ01-03 | 5/58 | 0/32 | 0.1043 | 276 |
| TCRBV16-01 CASSPGTGVNQAPLF TCRBJ01-05 | 5/58 | 0/32 | 0.1043 | 277 |
| TCRBV02-01 CASSQDGQYAEQFF TCRBJ02-01 | 5/58 | 0/32 | 0.1043 | 278 |
| TCRBV02-01 CASSQGLGVSYEQYF TCRBJ02-07 | 5/58 | 0/32 | 0.1043 | 279 |
| TCRBV02-01 CASSRTGSAETLYF TCRBJ02-03 | 5/58 | 0/32 | 0.1043 | 280 |
| TCRBV16-01 CASSSLSYEQYF TCRBJ02-07 | 5/58 | 0/32 | 0.1043 | 281 |
| TCRBV20-01 CGAGTNNNQAPLF TCRBJ01-05 | 5/58 | 0/32 | 0.1043 | 282 |
| TCRBV01-01 CTCSADLGSDYTF TCRBJ01-02 | 5/58 | 0/32 | 0.1043 | 283 |
| TCRBV13-02 CASGVDSYEQYF TCRBJ02-07 | 5/58 | 0/32 | 0.1043 | 284 |
| TCRBV13-03 CASSEGQGYAEQFF TCRBJ02-01 | 5/58 | 0/32 | 0.1043 | 285 |
| TCRBV03-01 CASSFQGAYEQYF TCRBJ02-07 | 5/58 | 0/32 | 0.1043 | 286 |
| TCRBV19-01 CASSGTTNSDYTF TCRBJ01-02 | 5/58 | 0/32 | 0.1043 | 287 |
| TCRBV12-01 CASSLGGSNSDYTF TCRBJ01-02 | 5/58 | 0/32 | 0.1043 | 288 |
| TCRBV26-01 CASSLSRNNQAPLF TCRBJ01-05 | 5/58 | 0/32 | 0.1043 | 289 |
| TCRBV19-01 CASSMGRAGNTLYF TCRBJ01-03 | 5/58 | 0/32 | 0.1043 | 290 |
| TCRBV15-01 CASSPDRNYAEQFF TCRBJ02-01 | 5/58 | 0/32 | 0.1043 | 291 |
| TCRBV16-01 CASSPGQNERLFF TCRBJ01-04 | 5/58 | 0/32 | 0.1043 | 292 |
| TCRBV15-01 CASSPGQSYEQYF TCRBJ02-07 | 5/58 | 0/32 | 0.1043 | 293 |
| TCRBV16-01 CASSPTISNERLFF TCRBJ01-04 | 5/58 | 0/32 | 0.1043 | 294 |
| TCRBV02-01 CASSQDGQGSYEQYF TCRBJ02-07 | 5/58 | 0/32 | 0.1043 | 295 |
| TCRBV02-01 CASSQEQANSDYTF TCRBJ01-02 | 5/58 | 0/32 | 0.1043 | 296 |
| TCRBV02-01 CASSQGHISNERLFF TCRBJ01-04 | 5/58 | 0/32 | 0.1043 | 297 |
| TCRBV14-01 CASSYSQNTLYF TCRBJ02-04 | 5/58 | 0/32 | 0.1043 | 298 |
| TCRBV19-01 CASTRDSSGNTLYF TCRBJ01-03 | 5/58 | 0/32 | 0.1043 | 299 |
| TCRBV31-01 CAWSLPNSGNTLYF TCRBJ01-03 | 5/58 | 0/32 | 0.1043 | 300 |
| TCRBV13-02 CASGDGRDEQYF TCRBJ02-07 | 5/58 | 0/32 | 0.1043 | 301 |
| TCRBV13-02 CASGEGGNSGNTLYF TCRBJ01-03 | 5/58 | 0/32 | 0.1043 | 302 |
| TCRBV13-02 CASGQGANERLFF TCRBJ01-04 | 5/58 | 0/32 | 0.1043 | 303 |
| TCRBV13-03 CASRTTNSDYTF TCRBJ01-02 | 5/58 | 0/32 | 0.1043 | 304 |
| TCRBV13-01 CASSDADRDEQYF TCRBJ02-07 | 5/58 | 0/32 | 0.1043 | 305 |
| TCRBV13-01 CASSDARGRDTQYF TCRBJ02-05 | 5/58 | 0/32 | 0.1043 | 306 |
| TCRBV04-01 CASSHRGGNQAPLF TCRBJ01-05 | 5/58 | 0/32 | 0.1043 | 307 |
| TCRBV12-01 CASSLAGGGSYEQYF TCRBJ02-07 | 5/58 | 0/32 | 0.1043 | 308 |
| TCRBV04-01 CASSLDISGNTLYF TCRBJ01-03 | 5/58 | 0/32 | 0.1043 | 309 |
| TCRBV03-01 CASSLEGGDSDYTF TCRBJ01-02 | 5/58 | 0/32 | 0.1043 | 310 |
| TCRBV16-01 CASSLGGPEQYF TCRBJ02-07 | 5/58 | 0/32 | 0.1043 | 311 |
| TCRBV12-01 CASSLGGPYAEQFF TCRBJ02-01 | 5/58 | 0/32 | 0.1043 | 312 |
| TCRBV12-02 CASSLTGGVEQYF TCRBJ02-07 | 5/58 | 0/32 | 0.1043 | 313 |
| TCRBV26-01 CASSPGLGGSYEQYF TCRBJ02-07 | 5/58 | 0/32 | 0.1043 | 314 |
| TCRBV02-01 CASSQDGVSGNTLYF TCRBJ01-03 | 5/58 | 0/32 | 0.1043 | 315 |
| TCRBV05-01 CASSQEGGVEQYF TCRBJ02-07 | 5/58 | 0/32 | 0.1043 | 316 |
| TCRBV16-01 CASSSGTGGGYEQYF TCRBJ02-07 | 5/58 | 0/32 | 0.1043 | 317 |
| TCRBV31-01 CAWRQNSGNTLYF TCRBJ01-03 | 5/58 | 0/32 | 0.1043 | 318 |
| TCRBV31-01 CAWSLGTNSGNTLYF TCRBJ01-03 | 5/58 | 0/32 | 0.1043 | 319 |
| TCRBV31-01 CAWSLWGDEQYF TCRBJ02-07 | 5/58 | 0/32 | 0.1043 | 320 |
| TCRBV01-01 CTCSAATNERLFF TCRBJ01-04 | 5/58 | 0/32 | 0.1043 | 321 |
| TCRBV13-02 CASGARDNYAEQFF TCRBJ02-01 | 5/58 | 0/32 | 0.1043 | 322 |
| TCRBV13-02 CASGAYAEQFF TCRBJ02-01 | 5/58 | 0/32 | 0.1043 | 323 |
| TCRBV13-02 CASGDDTGGYEQYF TCRBJ02-07 | 5/58 | 0/32 | 0.1043 | 324 |
| TCRBV13-02 CASGEQFF TCRBJ02-01 | 5/58 | 0/32 | 0.1043 | 325 |
| TCRBV13-03 CASRDRNTGQLYF TCRBJ02-02 | 5/58 | 0/32 | 0.1043 | 326 |
| TCRBV13-01 CASSDAVSQNTLYF TCRBJ02-04 | 5/58 | 0/32 | 0.1043 | 327 |
| TCRBV13-01 CASSDLGDYAEQFF TCRBJ02-01 | 5/58 | 0/32 | 0.1043 | 328 |
| TCRBV14-01 CASSFGGNTLYF TCRBJ01-03 | 5/58 | 0/32 | 0.1043 | 329 |
| TCRBV04-01 CASSFQANSDYTF TCRBJ01-02 | 5/58 | 0/32 | 0.1043 | 330 |
| TCRBV04-01 CASSFRNSDYTF TCRBJ01-02 | 5/58 | 0/32 | 0.1043 | 331 |
| TCRBV12-02 CASSGGNYAEQFF TCRBJ02-01 | 5/58 | 0/32 | 0.1043 | 332 |
| TCRBV13-03 CASSGGQGSAETLYF TCRBJ02-03 | 5/58 | 0/32 | 0.1043 | 333 |
| TCRBV12-01 CASSHGLGGNYAEQFF TCRBJ02-01 | 5/58 | 0/32 | 0.1043 | 334 |
| TCRBV16-01 CASSLAGRTEVFF TCRBJ01-01 | 5/58 | 0/32 | 0.1043 | 335 |
| TCRBV03-01 CASSLDGGSYEQYF TCRBJ02-07 | 5/58 | 0/32 | 0.1043 | 336 |
| TCRBV12-01 CASSLLGGREQYF TCRBJ02-07 | 5/58 | 0/32 | 0.1043 | 337 |
| TCRBV03-01 CASSLLVNQDTQYF TCRBJ02-05 | 5/58 | 0/32 | 0.1043 | 338 |
| TCRBV13-01 CASSLQGYEQYF TCRBJ02-07 | 5/58 | 0/32 | 0.1043 | 339 |
| TCRBV19-01 CASSLRGSGNTLYF TCRBJ01-03 | 5/58 | 0/32 | 0.1043 | 340 |
| TCRBV26-01 CASSLSVNSGNTLYF TCRBJ01-03 | 5/58 | 0/32 | 0.1043 | 341 |
| TCRBV12-01 CASSLWGDEQYF TCRBJ02-07 | 5/58 | 0/32 | 0.1043 | 342 |
| TCRBV12-02 CASSPTSSAETLYF TCRBJ02-03 | 5/58 | 0/32 | 0.1043 | 343 |
| TCRBV02-01 CASSQDGQDTQYF TCRBJ02-05 | 5/58 | 0/32 | 0.1043 | 344 |
| TCRBV05-01 CASSQEEGGEQYF TCRBJ02-07 | 5/58 | 0/32 | 0.1043 | 345 |
| TCRBV02-01 CASSRDRGREQYF TCRBJ02-07 | 5/58 | 0/32 | 0.1043 | 346 |
| TCRBV16-01 CASSRTTNSDYTF TCRBJ01-02 | 5/58 | 0/32 | 0.1043 | 347 |
| TCRBV04-01 CASSSDRVGNTLYF TCRBJ01-03 | 5/58 | 0/32 | 0.1043 | 348 |
| TCRBV16-01 CASSSGLGGENTLYF TCRBJ02-04 | 5/58 | 0/32 | 0.1043 | 349 |
| TCRBV03-01 CASSSGTSNSDYTF TCRBJ01-02 | 5/58 | 0/32 | 0.1043 | 350 |
| TCRBV31-01 CAWSLEGDTQYF TCRBJ02-05 | 5/58 | 0/32 | 0.1043 | 351 |
| TCRBV31-01 CAWSLSGGARAEQFF TCRBJ02-01 | 5/58 | 0/32 | 0.1043 | 352 |
| TCRBV20-01 CGARVGQNSDYTF TCRBJ01-02 | 5/58 | 0/32 | 0.1043 | 353 |
| TCRBV01-01 CTCSAGGAPEQYF TCRBJ02-07 | 5/58 | 0/32 | 0.1043 | 354 |
| TCRBV13-02 CASGDAGAEDTQYF TCRBJ02-05 | 5/58 | 0/32 | 0.1043 | 355 |
| TCRBV13-02 CASGERLGVNQDTQYF TCRBJ02-05 | 5/58 | 0/32 | 0.1043 | 356 |
| TCRBV13-02 CASGETGAQDTQYF TCRBJ02-05 | 5/58 | 0/32 | 0.1043 | 357 |
| TCRBV13-03 CASRTSSAETLYF TCRBJ02-03 | 5/58 | 0/32 | 0.1043 | 358 |
| TCRBV13-01 CASSDADIQDTQYF TCRBJ02-05 | 5/58 | 0/32 | 0.1043 | 359 |
| TCRBV13-01 CASSDALNTEVFF TCRBJ01-01 | 5/58 | 0/32 | 0.1043 | 360 |
| TCRBV13-03 CASSDRETLYF TCRBJ02-03 | 5/58 | 0/32 | 0.1043 | 361 |
| TCRBV13-03 CASSDRGPNTGQLYF TCRBJ02-02 | 5/58 | 0/32 | 0.1043 | 362 |
| TCRBV13-03 CASSERQNTLYF TCRBJ02-04 | 5/58 | 0/32 | 0.1043 | 363 |
| TCRBV12-01 CASSGDSAETLYF TCRBJ02-03 | 5/58 | 0/32 | 0.1043 | 364 |
| TCRBV19-01 CASSIGRNQDTQYF TCRBJ02-05 | 5/58 | 0/32 | 0.1043 | 365 |
| TCRBV03-01 CASSLEGQNYAEQFF TCRBJ02-01 | 5/58 | 0/32 | 0.1043 | 366 |
| TCRBV03-01 CASSLEGRNTGQLYF TCRBJ02-02 | 5/58 | 0/32 | 0.1043 | 367 |
| TCRBV03-01 CASSLGFNQDTQYF TCRBJ02-05 | 5/58 | 0/32 | 0.1043 | 368 |
| TCRBV12-02 CASSLGGAAETLYF TCRBJ02-03 | 5/58 | 0/32 | 0.1043 | 369 |
| TCRBV12-01 CASSLGGGGAEQFF TCRBJ02-01 | 5/58 | 0/32 | 0.1043 | 370 |
| TCRBV15-01 CASSLGTTNTGQLYF TCRBJ02-02 | 5/58 | 0/32 | 0.1043 | 371 |
| TCRBV12-01 CASSLLGGRDTQYF TCRBJ02-05 | 5/58 | 0/32 | 0.1043 | 372 |
| TCRBV03-01 CASSLLNQDTQYF TCRBJ02-05 | 5/58 | 0/32 | 0.1043 | 373 |
| TCRBV12-02 CASSPDSSAETLYF TCRBJ02-03 | 5/58 | 0/32 | 0.1043 | 374 |
| TCRBV03-01 CASSPDWGDTGQLYF TCRBJ02-02 | 5/58 | 0/32 | 0.1043 | 375 |
| TCRBV02-01 CASSQAANTEVFF TCRBJ01-01 | 5/58 | 0/32 | 0.1043 | 376 |
| TCRBV02-01 CASSQDHSSGNTLYF TCRBJ01-03 | 5/58 | 0/32 | 0.1043 | 377 |
| TCRBV02-01 CASSQEGGRGAETLYF TCRBJ02-03 | 5/58 | 0/32 | 0.1043 | 378 |
| TCRBV02-01 CASSQGRGAETLYF TCRBJ02-03 | 5/58 | 0/32 | 0.1043 | 379 |
| TCRBV02-01 CASSQLGSSAETLYF TCRBJ02-03 | 5/58 | 0/32 | 0.1043 | 380 |
| TCRBV02-01 CASSQPGANTEVFF TCRBJ01-01 | 5/58 | 0/32 | 0.1043 | 381 |
| TCRBV04-01 CASSRDRNYAEQFF TCRBJ02-01 | 5/58 | 0/32 | 0.1043 | 382 |
| TCRBV16-01 CASSRQGTEVFF TCRBJ01-01 | 5/58 | 0/32 | 0.1043 | 383 |
| TCRBV31-01 CAWSLDTLYF TCRBJ02-04 | 5/58 | 0/32 | 0.1043 | 384 |
| TCRBV01-01 CTCSAGDSPLYF TCRBJ01-06 | 5/58 | 0/32 | 0.1043 | 385 |
| TCRBV01-01 CTCSAGQGADTEVFF TCRBJ01-01 | 5/58 | 0/32 | 0.1043 | 386 |
| TCRBV01-01 CTCSAGVNSPLYF TCRBJ01-06 | 5/58 | 0/32 | 0.1043 | 387 |
| TCRBV13-02 CASGDAGGTQDTQYF TCRBJ02-05 | 5/58 | 0/32 | 0.1043 | 388 |
| TCRBV13-02 CASGDAGGVSQNTLYF TCRBJ02-04 | 5/58 | 0/32 | 0.1043 | 389 |
| TCRBV13-02 CASGDAGRDTEVFF TCRBJ01-01 | 5/58 | 0/32 | 0.1043 | 390 |
| TCRBV13-02 CASGDDWGGTGQLYF TCRBJ02-02 | 5/58 | 0/32 | 0.1043 | 391 |
| TCRBV13-02 CASGDTGQNTLYF TCRBJ02-04 | 5/58 | 0/32 | 0.1043 | 392 |
| TCRBV13-02 CASGEGTGGANTEVFF TCRBJ01-01 | 5/58 | 0/32 | 0.1043 | 393 |
| TCRBV13-02 CASGQGASAETLYF TCRBJ02-03 | 5/58 | 0/32 | 0.1043 | 394 |
| TCRBV13-03 CASRGTGDTEVFF TCRBJ01-01 | 5/58 | 0/32 | 0.1043 | 395 |
| TCRBV13-03 CASSAGTTNTEVFF TCRBJ01-01 | 5/58 | 0/32 | 0.1043 | 396 |
| TCRBV13-01 CASSDATGASQNTLYF TCRBJ02-04 | 5/58 | 0/32 | 0.1043 | 397 |
| TCRBV04-01 CASSFTGGDTEVFF TCRBJ01-01 | 5/58 | 0/32 | 0.1043 | 398 |
| TCRBV02-01 CASSHGQNTEVFF TCRBJ01-01 | 5/58 | 0/32 | 0.1043 | 399 |
| TCRBV19-01 CASSKGQNTGQLYF TCRBJ02-02 | 5/58 | 0/32 | 0.1043 | 400 |
| TCRBV03-01 CASSLASAETLYF TCRBJ02-03 | 5/58 | 0/32 | 0.1043 | 401 |
| TCRBV03-01 CASSLDWGGREQYF TCRBJ02-07 | 5/58 | 0/32 | 0.1043 | 402 |
| TCRBV03-01 CASSLEEDTQYF TCRBJ02-05 | 5/58 | 0/32 | 0.1043 | 403 |
| TCRBV12-02 CASSLEGGSSYEQYF TCRBJ02-07 | 5/58 | 0/32 | 0.1043 | 404 |
| TCRBV16-01 CASSLEGSSAETLYF TCRBJ02-03 | 5/58 | 0/32 | 0.1043 | 405 |
| TCRBV04-01 CASSLGHNTEVFF TCRBJ01-01 | 5/58 | 0/32 | 0.1043 | 406 |
| TCRBV12-01 CASSLGSYNSPLYF TCRBJ01-06 | 5/58 | 0/32 | 0.1043 | 407 |
| TCRBV12-02 CASSLGTGSAETLYF TCRBJ02-03 | 5/58 | 0/32 | 0.1043 | 408 |
| TCRBV16-01 CASSLGVQDTQYF TCRBJ02-05 | 5/58 | 0/32 | 0.1043 | 409 |
| TCRBV19-01 CASSLRDWGNTGQLYF TCRBJ02-02 | 5/58 | 0/32 | 0.1043 | 410 |
| TCRBV15-01 CASSLRGSAETLYF TCRBJ02-03 | 5/58 | 0/32 | 0.1043 | 411 |
| TCRBV12-01 CASSLRVNQDTQYF TCRBJ02-05 | 5/58 | 0/32 | 0.1043 | 412 |
| TCRBV29-01 CASSLSGQGNTEVFF TCRBJ01-01 | 5/58 | 0/32 | 0.1043 | 413 |
| TCRBV03-01 CASSLVGDAETLYF TCRBJ02-03 | 5/58 | 0/32 | 0.1043 | 414 |
| TCRBV19-01 CASSMGTTNTEVFF TCRBJ01-01 | 5/58 | 0/32 | 0.1043 | 415 |
| TCRBV13-03 CASSPNTEVFF TCRBJ01-01 | 5/58 | 0/32 | 0.1043 | 416 |
| TCRBV03-01 CASSPTGNTEVFF TCRBJ01-01 | 5/58 | 0/32 | 0.1043 | 417 |
| TCRBV05-01 CASSQAGGASAETLYF TCRBJ02-03 | 5/58 | 0/32 | 0.1043 | 418 |
| TCRBV02-01 CASSQEGGRNTLYF TCRBJ02-04 | 5/58 | 0/32 | 0.1043 | 419 |
| TCRBV05-01 CASSQEGQGNSDYTF TCRBJ01-02 | 5/58 | 0/32 | 0.1043 | 420 |
| TCRBV05-01 CASSQELGDYAEQFF TCRBJ02-01 | 5/58 | 0/32 | 0.1043 | 421 |
| TCRBV02-01 CASSQGGGDTQYF TCRBJ02-05 | 5/58 | 0/32 | 0.1043 | 422 |
| TCRBV05-01 CASSQRDTEVFF TCRBJ01-01 | 5/58 | 0/32 | 0.1043 | 423 |
| TCRBV04-01 CASSRDWGGTGQLYF TCRBJ02-02 | 5/58 | 0/32 | 0.1043 | 424 |
| TCRBV19-01 CASSRTGGDDTQYF TCRBJ02-05 | 5/58 | 0/32 | 0.1043 | 425 |
| TCRBV19-01 CASSRTSSQNTLYF TCRBJ02-04 | 5/58 | 0/32 | 0.1043 | 426 |
| TCRBV13-01 CASSVQGNTEVFF TCRBJ01-01 | 5/58 | 0/32 | 0.1043 | 427 |
| TCRBV31-01 CAWSGQGANTEVFF TCRBJ01-01 | 5/58 | 0/32 | 0.1043 | 428 |
| TCRBV31-01 CAWSLGDRGDERLFF TCRBJ01-04 | 5/58 | 0/32 | 0.1043 | 429 |
| TCRBV31-01 CAWSLGGAEDTQYF TCRBJ02-05 | 5/58 | 0/32 | 0.1043 | 430 |
| TCRBV20-01 CGARGTGGSDYTF TCRBJ01-02 | 5/58 | 0/32 | 0.1043 | 431 |
| TCRBV20-01 CGASRNTEVFF TCRBJ01-01 | 5/58 | 0/32 | 0.1043 | 432 |
| TCRBV01-01 CTCSADRGVEVFF TCRBJ01-01 | 5/58 | 0/32 | 0.1043 | 433 |
| TCRBV01-01 CTCSAESSAETLYF TCRBJ02-03 | 5/58 | 0/32 | 0.1043 | 434 |
| TCRBV01-01 CTCSAVGGDTQYF TCRBJ02-05 | 5/58 | 0/32 | 0.1043 | 435 |

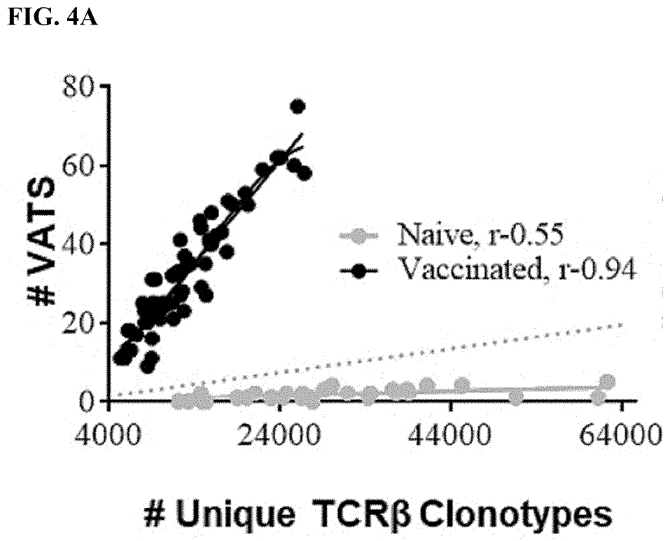

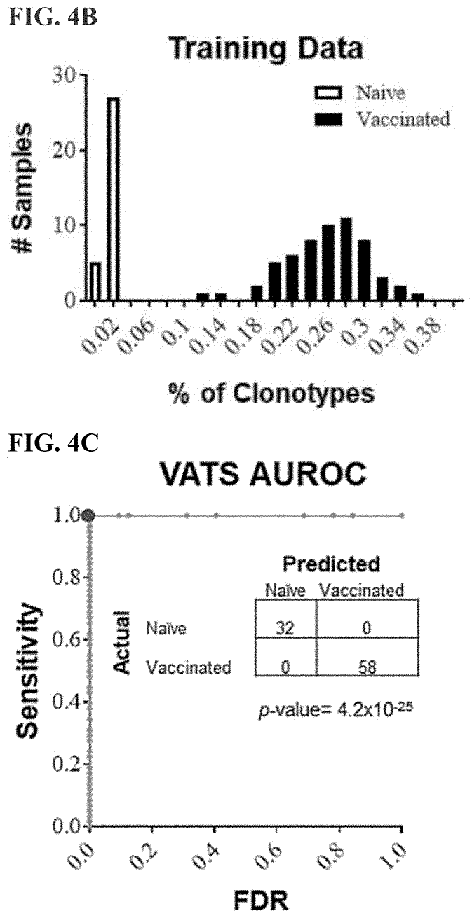

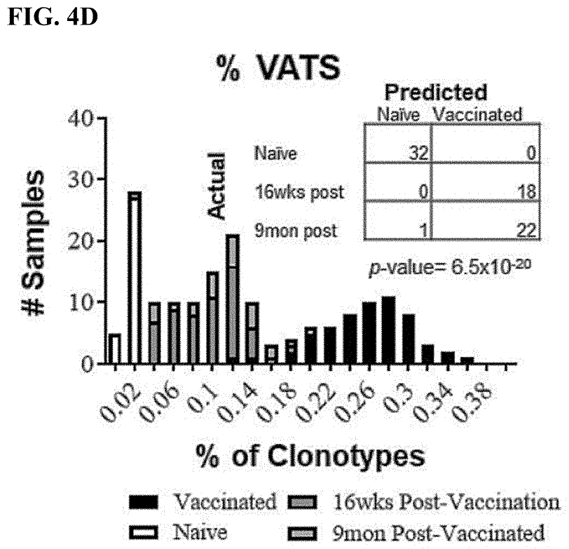

A diagnostic classifier was developed by calculating the number of VATS present relative to the total number of unique TCRβ clonotypes present for each sample. It was observed that the number of VATS present in a sample was significantly correlated with the total number of unique TCRβ clonotypes in both vaccinated and naive samples, indicating that the number of TCRβ clonotypes present directly affects the number of VATS identified (FIG. 4A). To normalize for differences in the number of TCRβ clonotypes identified in various samples, comparisons between naive and vaccinated samples were analyzed in the context of % VATS, the proportion of VATS in an individual sample. This calculation is displayed as a percentage of all unique TCRβ clonotypes:

$$\frac{\# VATS present}{\# Unique TCR β clonotypes}$$

A binary classification system was constructed to differentiate naive and vaccinated samples on the basis of the normal distribution of % VATS from the TCR repertoires of the naive or vaccinated groups. In this way, the TCR repertoires from the vaccinated and naive samples act as “training data,” teaching the diagnostic classifier to predict the vaccination status of samples (See the Materials and Methods provided before Example 1 herein).

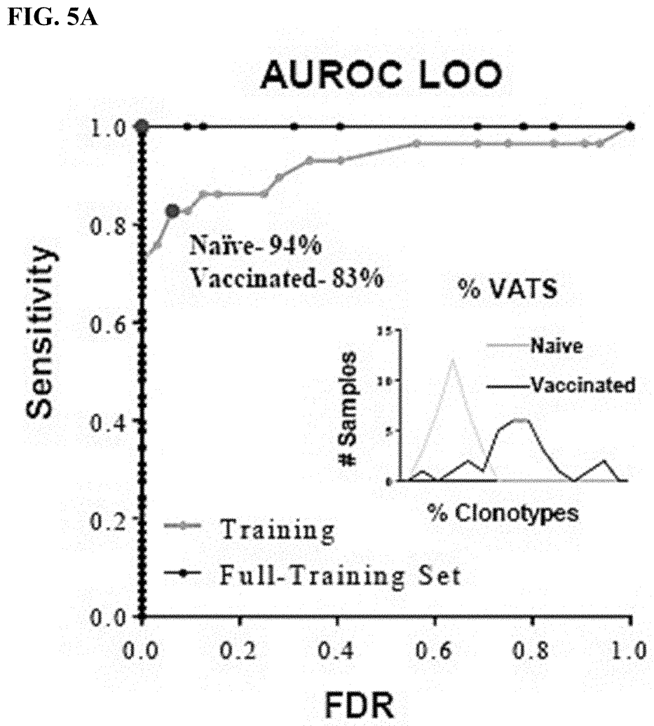

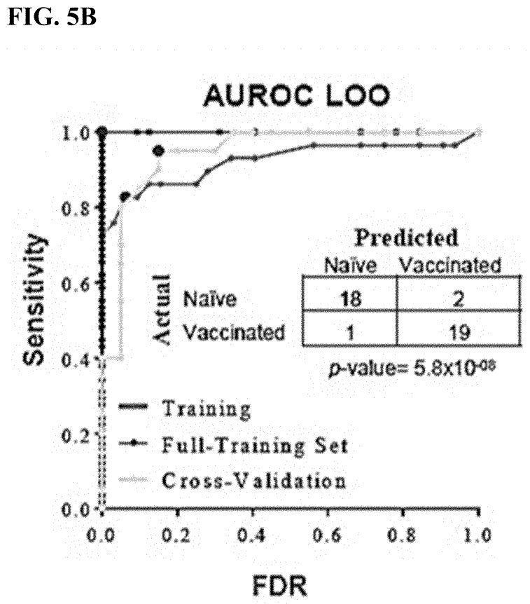

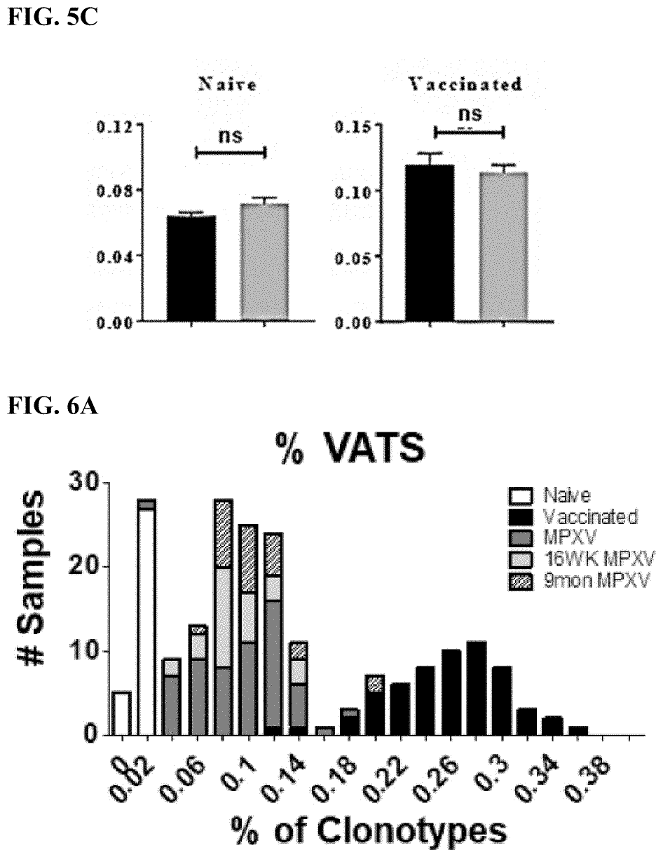

A comparison of naive and smallpox-vaccinated samples showed a 43-fold increase in the % VATS in vaccinated repertoires (average 0.248±0.047%) compared with naive repertoires (average 0.0057±0.0039%) (FIG. 4B). The diagnostic classifier of 315 TCRβ sequences correctly classified 100% (32 of 32) of naive samples and 100% (58 of 58) of the vaccinated samples (FIG. 4C). To determine whether the results of the training data were over-fitted from using the complete training dataset to inform the diagnostic classifier, an exhaustive leave-one-out (LOO) analysis was performed. All samples associated with an individual mouse (pre- and post-vaccination) were removed from the training data, and the VATS library was redefined for each individual mouse (average 308±30 clonotypes per library). The same methodology described previously was used to train a diagnostic assay and test the accuracy of the classifier using the sample(s) associated with the mouse left out. Overall, the LOO analysis showed that the diagnostic assay correctly classified 94% of naive samples and 83% of vaccinated samples (FIG. 5A). To confirm data from the LOO analysis, TCRβ repertoires from an independent cohort of HLA-A2 mice (n=20) not used in the construction of the VATS library or training of the diagnostic classifier were analyzed before and after vaccination with the ACAM2000 smallpox vaccine. We show that the diagnostic classifier correctly identified 90% (18 of 20) of naive and 95% (19 of 20) of vaccinated samples, and analysis of % VATS reveals no significant difference between training and cross-validation samples (FIG. 5B and FIG. 5C). Additionally, a cohort of HLA-A2 mice (n=15) infected with an unrelated virus (Zika virus) were distinguished from the smallpox-vaccinated mice with 93% accuracy. Overall, the training and cross-validation data from the LOO analysis closely resembles the results from the full-training set using the full VATS library of 315 TCR sequences.

To test whether the VATS diagnostic classifier was capable of identifying the vaccine-specific T cell response after the primary infection and generation of long-term memory, blood collected 16 weeks and 9 months after vaccination was analyzed. Sixteen week and 9-month post-vaccination samples were collected from the same mice 2 and 8 week samples used for the training data. TCRβ sequences from 16 week and 9 month post-vaccination samples were not used to generate the VATS library or as part of the training data. Enrichment of VATS was observed in the 16 week and 9 month post-vaccinated samples. The VATS library occupied on average 0.091±0.019% and 0.105±0.043% of TCRβ sequences from 16 week and 9 month post-vaccination samples, respectively, compared with naive repertoires (0.0057±0.0039%). Compared with the determination threshold calculated by the diagnostic assay, 100% of 16 week (18 of 18) and 96% of 9 month (22 of 23) post-vaccinated samples were correctly differentiated from naive samples (FIG. 4D). These data show that computational assessment of the TCRβ repertoire is capable of tracking the low-frequency virus-specific long-term memory population within the circulating T cell pool.

### Example 3: MPXV-Infected Mice are Distinguished from Naive Samples Using VATS Library

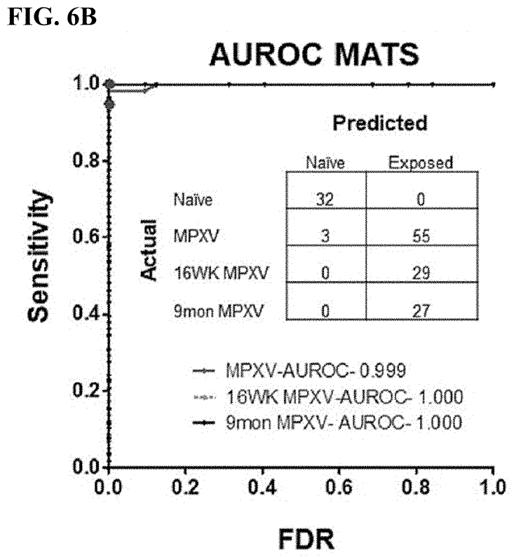

The accuracy of the VATS library and diagnostic classifier was tested using an unrelated cohort of mice infected with a highly related Orthopoxvirus, MPXV (FIG. 1C and FIG. 1D). The percentage of sequences in the post-MPXV samples matching sequences from the VATS library was calculated to determine if the vaccine-associated diagnostic assay could distinguish between the naive and MPXV-infected samples. % VATS was significantly increased (p<6.3×10⁻²⁴) in samples from mice 2 and 8 weeks post-MPXV infection (0.084±0.035%) compared with the 32 naive samples (0.0057±0.0039%). Additionally, % VATS was significantly increased in samples from mice 16 weeks (0.08±0.026%, p 1.7×10⁻¹⁵) and 9 months (0.097±0.033%, p 1.5×10⁻¹³) after infection with MPXV compared with naive samples (FIG. 6A). The VATS library and diagnostic approach developed in the ACAM2000-vaccinated mice correctly distinguished 55 of 58 (95%) 2 and 8 week MPXV-infected mice from naive samples as well as 29 of 29 (100%) and 27 of 27 (100%) samples from mice 16 weeks and 9 months after MPXV infection (FIG. 6B). The data show that the diagnostic assay is a robust platform, not only distinguishing ACAM2000-vaccinated mice from naive but also an independent cohort of mice infected with a highly related virus (95% identical) (Shchelkunov et al., 2002).

### Example 4: Cross-Validation of Diagnostic Classification System Through Identification of MPXV-Associated TCRβ Sequences

To determine whether the platform used to generate the VATS could be replicated independent of the ACAM2000 analysis, the same protocol was used with the TCRβ sequences identified in the MPXV-infected mice to generate a separate library of MPXV-associated TCR sequences (MATS). A total of 120 MATS were identified (Table 7).

TABLE 7
TCRβ alleles associated with monkey pox infection in mice

| | | | | CDR3 |
|---|---|---|---|---|
| | # Vaccinated | # Naïve | | SEQ ID |
| V-CDR3-J | Samples | Samples | p-value | NO: |
| TCRBV13-01 CASSDPGLGDYEQYF TCRBJ02-07 | 11/58 | 0/32 | 0.005473 | 1 |
| TCRBV14-01 CASSSTGYNNQAPLF TCRBJ01-05 | 8/58 | 0/32 | 0.024728 | 2 |
| TCRBV01-01 CTCSAEGGANTEVFF TCRBJ01-01 | 7/58 | 0/32 | 0.040243 | 3 |
| TCRBV04-01 CASSLGLGNYAEQFF TCRBJ02-01 | 7/58 | 0/32 | 0.040243 | 4 |
| TCRBV04-01 CASSLTGGNTEVFF TCRBJ01-01 | 7/58 | 0/32 | 0.040243 | 5 |
| TCRBV05-01 CASSPRDREDTQYF TCRBJ02-05 | 7/58 | 0/32 | 0.040243 | 6 |
| TCRBV02-01 CASSPDRDEQYF TCRBJ02-07 | 6/58 | 0/32 | 0.065009 | 7 |
| TCRBV02-01 CASSQDGANTGQLYF TCRBJ02-02 | 6/58 | 0/32 | 0.065009 | 8 |
| TCRBV03-01 CASSLEQNQAPLF TCRBJ01-05 | 6/58 | 0/32 | 0.065009 | 9 |
| TCRBV03-01 CASSPTGNTEVFF TCRBJ01-01 | 6/58 | 0/32 | 0.065009 | 10 |
| TCRBV04-01 CASSRSYNSPLYF TCRBJ01-06 | 6/58 | 0/32 | 0.065009 | 11 |
| TCRBV05-01 CASSPGTEVFF TCRBJ01-01 | 6/58 | 0/32 | 0.065009 | 12 |
| TCRBV05-01 CASSQDITEVFF TCRBJ01-01 | 6/58 | 0/32 | 0.065009 | 13 |
| TCRBV05-01 CASSQDWVNYAEQFF TCRBJ02-01 | 6/58 | 0/32 | 0.065009 | 14 |
| TCRBV12-01 CASSLGETLYF TCRBJ02-03 | 6/58 | 0/32 | 0.065009 | 15 |
| TCRBV13-01 CASSDAGEEQYF TCRBJ02-07 | 6/58 | 0/32 | 0.065009 | 16 |
| TCRBV13-02 CASGAGGEDTQYF TCRBJ02-05 | 6/58 | 0/32 | 0.065009 | 17 |
| TCRBV13-02 CASGDTGAGNTLYF TCRBJ01-03 | 6/58 | 0/32 | 0.065009 | 18 |
| TCRBV13-02 CASGEGLGKDTQYF TCRBJ02-05 | 6/58 | 0/32 | 0.065009 | 19 |
| TCRBV13-02 CASGPTFNQDTQYF TCRBJ02-05 | 6/58 | 0/32 | 0.065009 | 20 |
| TCRBV14-01 CASSFTGGNNQAPLF TCRBJ01-05 | 6/58 | 0/32 | 0.065009 | 21 |
| TCRBV16-01 CASSLAGNERLFF TCRBJ01-04 | 6/58 | 0/32 | 0.065009 | 22 |
| TCRBV19-01 CASSIGTGGNTGQLYF TCRBJ02-02 | 6/58 | 0/32 | 0.065009 | 23 |
| TCRBV26-01 CASSLRGTGNTLYF TCRBJ01-03 | 6/58 | 0/32 | 0.065009 | 24 |
| TCRBV26-01 CASSLTGGSNERLFF TCRBJ01-04 | 6/58 | 0/32 | 0.065009 | 25 |
| TCRBV29-01 CASSLRDIYEQYF TCRBJ02-07 | 6/58 | 0/32 | 0.065009 | 26 |
| TCRBV31-01 CAWSLDRYNSPLYF TCRBJ01-06 | 6/58 | 0/32 | 0.065009 | 27 |
| TCRBV31-01 CAWSLPNSGNTLYF TCRBJ01-03 | 6/58 | 0/32 | 0.065009 | 28 |
| TCRBV01-01 CTCSAAGTGVGNTLYF TCRBJ01-03 | 5/58 | 0/32 | 0.104259 | 29 |
| TCRBV01-01 CTCSADRGSYEQYF TCRBJ02-07 | 5/58 | 0/32 | 0.104259 | 30 |
| TCRBV01-01 CTCSAEDWGNYAEQFF TCRBJ02-01 | 5/58 | 0/32 | 0.104259 | 31 |
| TCRBV01-01 CTCSAGGSNTEVFF TCRBJ01-01 | 5/58 | 0/32 | 0.104259 | 32 |
| TCRBV01-01 CTCSAGRNSPLYF TCRBJ01-06 | 5/58 | 0/32 | 0.104259 | 33 |
| TCRBV01-01 CTCSARTGGAGEQYF TCRBJ02-07 | 5/58 | 0/32 | 0.104259 | 34 |
| TCRBV02-01 CASSQDGRGEQYF TCRBJ02-07 | 5/58 | 0/32 | 0.104259 | 35 |
| TCRBV02-01 CASSQDRTGNTEVFF TCRBJ01-01 | 5/58 | 0/32 | 0.104259 | 36 |
| TCRBV02-01 CASSQGGGTEVFF TCRBJ01-01 | 5/58 | 0/32 | 0.104259 | 37 |
| TCRBV03-01 CASSFQANTEVFF TCRBJ01-01 | 5/58 | 0/32 | 0.104259 | 38 |
| TCRBV03-01 CASSLARGYEQYF TCRBJ02-07 | 5/58 | 0/32 | 0.104259 | 39 |
| TCRBV03-01 CASSLDSSNTEVFF TCRBJ01-01 | 5/58 | 0/32 | 0.104259 | 40 |
| TCRBV03-01 CASSLGQGGGNTLYF TCRBJ01-03 | 5/58 | 0/32 | 0.104259 | 41 |
| TCRBV03-01 CASSLKGQDTQYF TCRBJ02-05 | 5/58 | 0/32 | 0.104259 | 42 |
| TCRBV03-01 CASSLSANTEVFF TCRBJ01-01 | 5/58 | 0/32 | 0.104259 | 43 |
| TCRBV03-01 CASSQTGGAREQYF TCRBJ02-07 | 5/58 | 0/32 | 0.104259 | 44 |
| TCRBV03-01 CASSYRNTEVFF TCRBJ01-01 | 5/58 | 0/32 | 0.104259 | 45 |
| TCRBV04-01 CASRTISNERLF TCRBJ01-04 | 5/58 | 0/32 | 0.104259 | 46 |
| TCRBV04-01 CASSFDRGEVFF TCRBJ01-01 | 5/58 | 0/32 | 0.104259 | 47 |
| TCRBV04-01 CASSPDWGGNTGQLYF TCRBJ02-02 | 5/58 | 0/32 | 0.104259 | 48 |
| TCRBV04-01 CASSPLGVNQDTQYF TCRBJ02-05 | 5/58 | 0/32 | 0.104259 | 49 |
| TCRBV04-01 CASSPTAYEQYF TCRBJ02-07 | 5/58 | 0/32 | 0.104259 | 50 |
| TCRBV05-01 CASSQEGQGGDTQYF TCRBJ02-05 | 5/58 | 0/32 | 0.104259 | 51 |
| TCRBV05-01 CASSQGDSSAETLYF TCRBJ02-03 | 5/58 | 0/32 | 0.104259 | 52 |
| TCRBV05-01 CASSQGLSNERLFF TCRBJ01-04 | 5/58 | 0/32 | 0.104259 | 53 |
| TCRBV05-01 CASSQLGGNTGQLYF TCRBJ02-02 | 5/58 | 0/32 | 0.104259 | 54 |
| TCRBV12-01 CASSGQSNERLFF TCRBJ01-04 | 5/58 | 0/32 | 0.104259 | 55 |
| TCRBV12-01 CASSLAGGGQNTLYF TCRBJ02-04 | 5/58 | 0/32 | 0.104259 | 56 |
| TCRBV12-01 CASSLPTNSDYTF TCRBJ01-02 | 5/58 | 0/32 | 0.104259 | 57 |
| TCRBV12-01 CASSLTGDYEQYF TCRBJ02-07 | 5/58 | 0/32 | 0.104259 | 58 |
| TCRBV12-01 CASSLTNQDTQYF TCRBJ02-05 | 5/58 | 0/32 | 0.104259 | 59 |
| TCRBV12-01 CASSWDWGSQNTLYF TCRBJ02-04 | 5/58 | 0/32 | 0.104259 | 60 |
| TCRBV12-02 CASSLEGGSSYEQYF TCRBJ02-07 | 5/58 | 0/32 | 0.104259 | 61 |
| TCRBV12-02 CASSLGLGVYAEQFF TCRBJ02-01 | 5/58 | 0/32 | 0.104259 | 62 |
| TCRBV12-02 CASSLRGNTLYF TCRBJ01-03 | 5/58 | 0/32 | 0.104259 | 63 |
| TCRBV12-02 CASSPDSGNTLYF TCRBJ01-03 | 5/58 | 0/32 | 0.104259 | 64 |
| TCRBV12-02 CASSPGQGSDYTF TCRBJ01-02 | 5/58 | 0/32 | 0.104259 | 65 |
| TCRBV13-01 CASRLGANTGQLYF TCRBJ02-02 | 5/58 | 0/32 | 0.104259 | 66 |
| TCRBV13-01 CASSDAGLGFYEQYF TCRBJ02-07 | 5/58 | 0/32 | 0.104259 | 67 |
| TCRBV13-01 CASSDAYSGNTLYF TCRBJ01-03 | 5/58 | 0/32 | 0.104259 | 68 |
| TCRBV13-01 CASSDPGLGFYEQYF TCRBJ02-07 | 5/58 | 0/32 | 0.104259 | 69 |
| TCRBV13-01 CASSDSANTGQLYF TCRBJ02-02 | 5/58 | 0/32 | 0.104259 | 70 |
| TCRBV13-01 CASSETGNYAEQFF TCRBJ02-01 | 5/58 | 0/32 | 0.104259 | 71 |
| TCRBV13-02 CASGAGAGNTLYF TCRBJ01-03 | 5/58 | 0/32 | 0.104259 | 72 |
| TCRBV13-02 CASGDAGEQDTQYF TCRBJ02-05 | 5/58 | 0/32 | 0.104259 | 73 |
| TCRBV13-02 CASGDARGENTLYF TCRBJ02-04 | 5/58 | 0/32 | 0.104259 | 74 |
| TCRBV13-02 CASGDFNSPLYF TCRBJ01-06 | 5/58 | 0/32 | 0.104259 | 75 |
| TCRBV13-02 CASGDRFSYEQYF TCRBJ02-07 | 5/58 | 0/32 | 0.104259 | 76 |
| TCRBV13-02 CASGEAGDYAEQFF TCRBJ02-01 | 5/58 | 0/32 | 0.104259 | 77 |
| TCRBV13-02 CASGPGQSNTEVFF TCRBJ01-01 | 5/58 | 0/32 | 0.104259 | 78 |
| TCRBV13-03 CASSDAGSNERLFF TCRBJ01-04 | 5/58 | 0/32 | 0.104259 | 79 |
| TCRBV13-03 CASSDATGGYEQYF TCRBJ02-07 | 5/58 | 0/32 | 0.104259 | 80 |
| TCRBV13-03 CASSGTGVSYEQYF TCRBJ02-07 | 5/58 | 0/32 | 0.104259 | 81 |
| TCRBV14-01 CASSFTGQNNQAPLF TCRBJ01-05 | 5/58 | 0/32 | 0.104259 | 82 |
| TCRBV14-01 CASSFTGRNNQAPLF TCRBJ01-05 | 5/58 | 0/32 | 0.104259 | 83 |
| TCRBV15-01 CASSLDKNTGQLYF TCRBJ02-02 | 5/58 | 0/32 | 0.104259 | 84 |
| TCRBV15-01 CASSLGVYEQYF TCRBJ02-07 | 5/58 | 0/32 | 0.104259 | 85 |
| TCRBV15-01 CASSLRGSGNTLYF TCRBJ01-03 | 5/58 | 0/32 | 0.104259 | 86 |
| TCRBV15-01 CASSPGQYAEQFF TCRBJ02-01 | 5/58 | 0/32 | 0.104259 | 87 |
| TCRBV16-01 CASSWGGNQDTQYF TCRBJ02-05 | 5/58 | 0/32 | 0.104259 | 88 |
| TCRBV17-01 CASSRRQYEQYF TCRBJ02-07 | 5/58 | 0/32 | 0.104259 | 89 |
| TCRBV19-01 CASSIRDWGGAEQFF TCRBJ02-01 | 5/58 | 0/32 | 0.104259 | 90 |
| TCRBV19-01 CASSLTGNNQAPLF TCRBJ01-05 | 5/58 | 0/32 | 0.104259 | 91 |
| TCRBV19-01 CASSMTGGSQNTLYF TCRBJ02-04 | 5/58 | 0/32 | 0.104259 | 92 |
| TCRBV19-01 CASSRDKQDTQYF TCRBJ02-05 | 5/58 | 0/32 | 0.104259 | 93 |
| TCRBV20-01 CGARDRGKNTLYF TCRBJ02-04 | 5/58 | 0/32 | 0.104259 | 94 |
| TCRBV20-01 CGARVGSAETLYF TCRBJ02-03 | 5/58 | 0/32 | 0.104259 | 95 |
| TCRBV23-01 CSSSQTNTGQLYF TCRBJ02-02 | 5/58 | 0/32 | 0.104259 | 96 |
| TCRBV26-01 CASSLQKNTEVFF TCRBJ01-01 | 5/58 | 0/32 | 0.104259 | 97 |
| TCRBV26-01 CASSLSRANSDYTF TCRBJ01-02 | 5/58 | 0/32 | 0.104259 | 98 |
| TCRBV26-01 CASSLYRAGNTLYF TCRBJ01-03 | 5/58 | 0/32 | 0.104259 | 99 |
| TCRBV26-01 CASSQDSYNSPLYF TCRBJ01-06 | 5/58 | 0/32 | 0.104259 | 100 |
| TCRBV26-01 CASSRGVSGNTLYF TCRBJ01-03 | 5/58 | 0/32 | 0.104259 | 101 |
| TCRBV29-01 CASSFGQGNTEVFF TCRBJ01-01 | 5/58 | 0/32 | 0.104259 | 102 |
| TCRBV29-01 CASSFGSNERLFF TCRBJ01-04 | 5/58 | 0/32 | 0.104259 | 103 |
| TCRBV29-01 CASSLGDSNERLFF TCRBJ01-04 | 5/58 | 0/32 | 0.104259 | 104 |
| TCRBV29-01 CASSLGTGYAEQFF TCRBJ02-01 | 5/58 | 0/32 | 0.104259 | 105 |
| TCRBV29-01 CASSLRDRNTGQLYF TCRBJ02-02 | 5/58 | 0/32 | 0.104259 | 106 |
| TCRBV29-01 CASSRQGANSDYTF TCRBJ01-02 | 5/58 | 0/32 | 0.104259 | 107 |
| TCRBV29-01 CASSSGTGSNERLFF TCRBJ01-04 | 5/58 | 0/32 | 0.104259 | 108 |
| TCRBV29-01 CASSTGTEVFF TCRBJ01-01 | 5/58 | 0/32 | 0.104259 | 109 |
| TCRBV31-01 CAWKGQSNSDYTF TCRBJ01-02 | 5/58 | 0/32 | 0.104259 | 110 |
| TCRBV31-01 CAWSLEGRDTQYF TCRBJ02-05 | 5/58 | 0/32 | 0.104259 | 111 |
| TCRBV31-01 CAWSPRDTQYF TCRBJ02-05 | 5/58 | 0/32 | 0.104259 | 112 |
| TCRBV31-01 CAWSQGGNSDYTF TCRBJ01-02 | 5/58 | 0/32 | 0.104259 | 113 |
| TCRBV12-01 CASSPGISNERLFF TCRBJ01-04 | 9/58 | 1/32 | 0.068689 | 114 |
| TCRBV02-01 CASSQGGNSDYTF TCRBJ01-02 | 8/58 | 1/32 | 0.101927 | 115 |
| TCRBV05-01 CASSQEGGVNQDTQYF TCRBJ02-05 | 8/58 | 1/32 | 0.101927 | 116 |
| TCRBV31-01 CAWSLGGVYEQYF TCRBJ02-07 | 8/58 | 1/32 | 0.101927 | 117 |
| TCRBV31-01 CAWSLQANTEVFF TCRBJ01-01 | 8/58 | 1/32 | 0.101927 | 118 |
| TCRBV04-01 CASSRDSQNTLYF TCRBJ02-04 | 15/58 | 2/32 | 0.018742 | 119 |
| TCRBV15-01 CASSLEGGNTEVFF TCRBJ01-01 | 11/58 | 2/32 | 0.088006 | 120 |

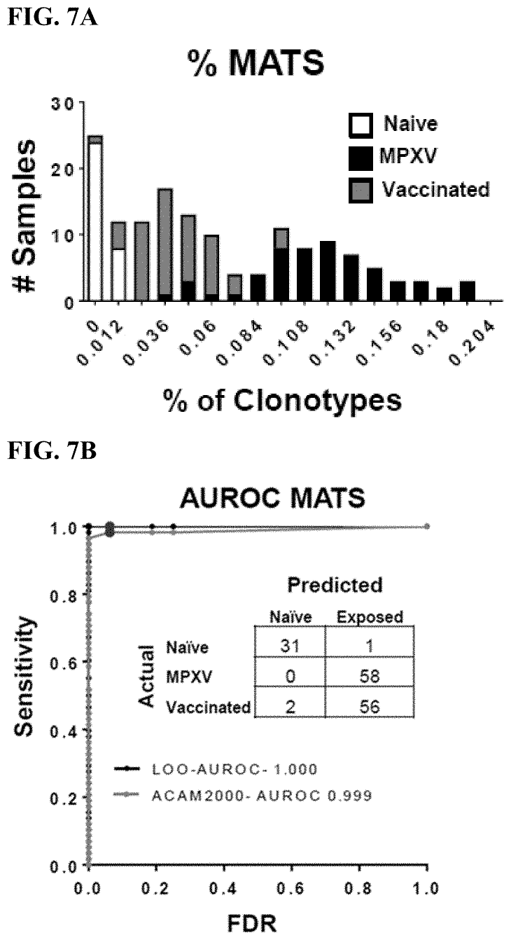

Using the same diagnostic approach implemented with the VATS library, a diagnostic classifier using the MATS library was generated. The proportion of a sample's unique TCRβ clonotypes occupied by MATS (% MATS) was calculated and used to distinguish between naïve and infected or vaccinated samples. Compared with naïve samples (0.0009+/−0.0018%), there were significant increases in the % MATS of MPXV-infected samples (0.114+/−0.037%, 126.7-fold increase) and ACAM2000-vaccinated samples (0.036%+/−0.02%, 40-fold increase, FIG. 7A). The diagnostic assay using the MATS library correctly classified 97% of the naïve samples (31 of 32), 100% of the MPXV-infected samples (58 of 58), and 97% of the ACAM2000 smallpox-vaccinated (56 of 58) samples (FIG. 7B). With these data, the methodology for producing a diagnostic assay capable of distinguishing between naïve and MPXV- or vaccine exposed samples with a high degree of accuracy was replicated using a large independent cohort of mice. This confirms that construction of pathogen-associated TCR libraries as diagnostic assays can be used as a highly robust, accurate, and reproducible methodology for the identification and tracking of vaccine- and pathogen-specific T cell populations.

### Example 5: The VATS Library Contains Virus-Specific TCR Sequences

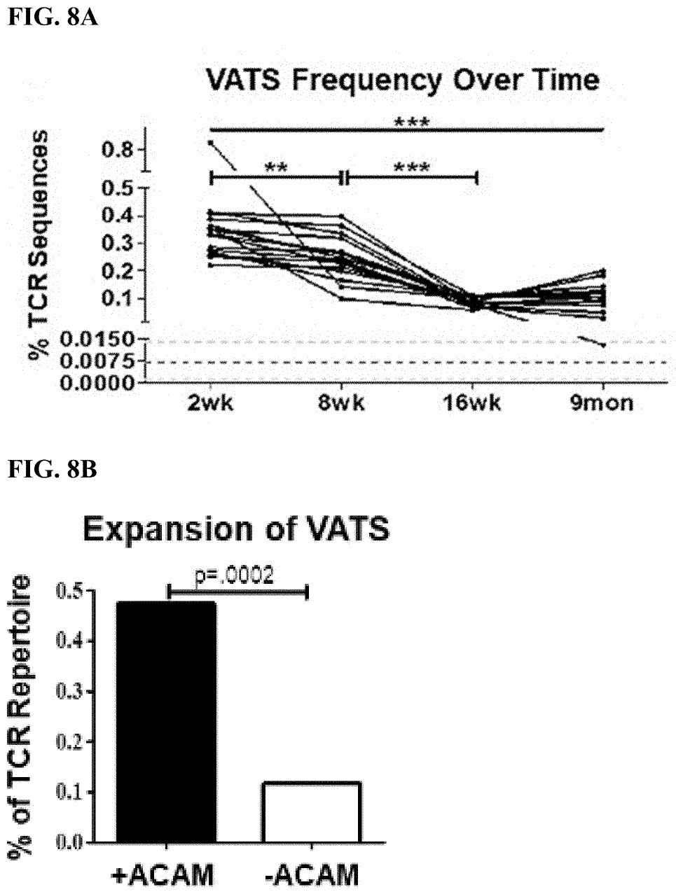

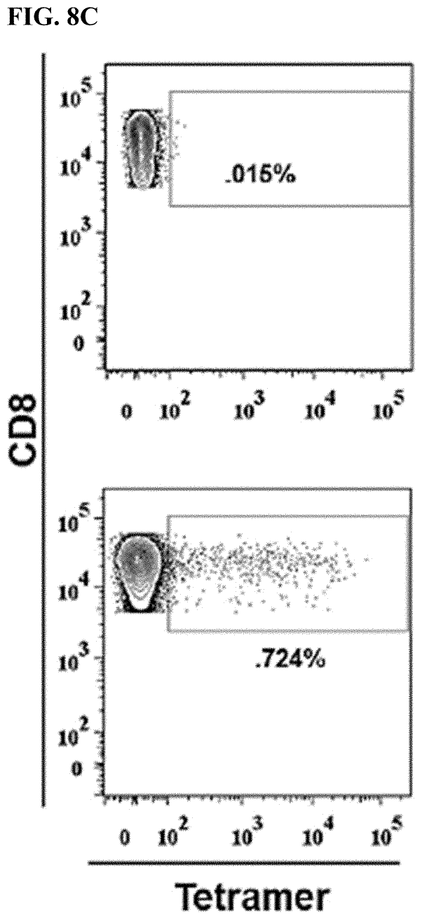

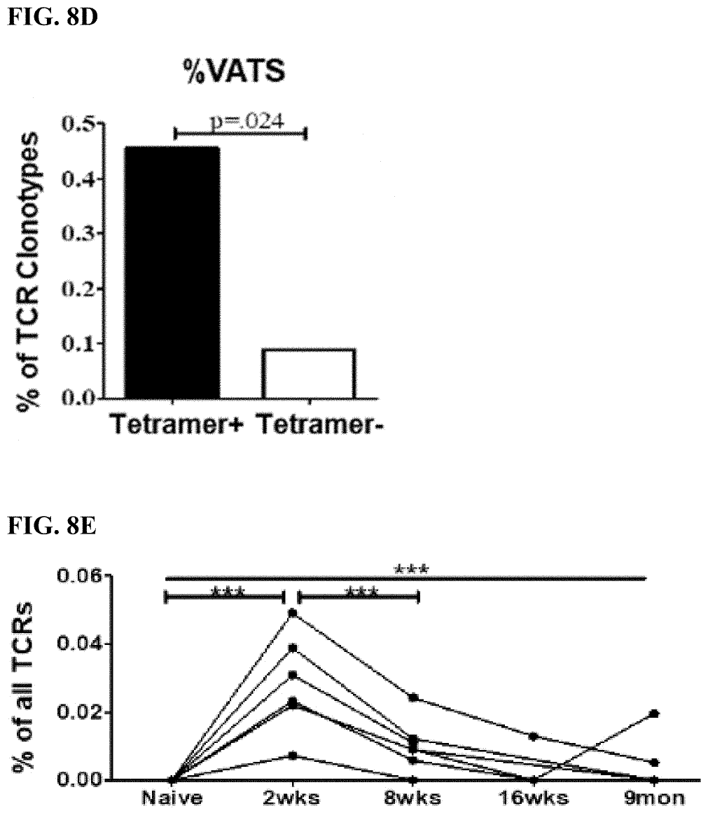

The diagnostic assay has shown the ability to monitor and track the presence of sequences from the VATS library over time in vaccinated or infected mice. Using sequence analyses, the relative frequencies of TCR sequences from the VATS library within the circulating T cell repertoire was determined. The frequencies of VATS sequences significantly decrease in mice over time from 2 weeks (0.35±0.13%) through 9 months (0.11±0.05%) after exposure (FIG. 8A). The decrease in frequency of VATS sequences over time is consistent with that of an antigen-specific response, displaying virus-specific T cells at higher frequencies early after vaccination (2 weeks) before declining in frequency and remaining as long-lived memory T cells (8 weeks, 16 weeks, and 9 months) (FIG. 8E). Example 2 (FIG. 3B) showed that in vitro stimulation of splenocytes from vaccinated mice with the ACAM2000 smallpox vaccine resulted in preferential expansion of TCR clonotypes associated with post-vaccinated samples. To determine if the VATS library contained virus-specific TCRs, splenocytes from vaccinated mice were cultured in vitro with or without ACAM2000 for 5 days to induce expansion of vaccine-specific T cells. The TCR repertoire from T cells cultured with ACAM2000 was analyzed for the expansion of sequences included in the VATS library compared with the untreated control. The frequencies of VATS were 4-fold higher after culture with ACAM2000, representing approximately 0.47% of all T cells sequenced compared with 0.12% in unstimulated controls (FIG. 8B). These data confirm that TCRs recognizing smallpox vaccine antigens are present in the VATS library.

TCR sequences identified in the VATS library were examined for sequences specific for known HLA-A2 epitopes previously identified in VACV-immune humans and HLA-A2 transgenic mice (Gilchuk et al., 2013). HLA-A2 tetramers loaded with nine different vaccinia peptides were used to identify and isolate HLA-A2-restricted vaccinia-specific T cells (Table 8). The vaccinia peptides loaded onto HLA-A2 tetramers had been previously shown by Gilchuk et al. (2013) to elicit strong CD8+ T cells responses in HLA-A2 transgenic mice. Mice approximately 6 months post-vaccination were boosted with ACAM2000; after 4 days, splenocytes were isolated, and tetramer-binding CD8+ T cells were isolated using fluorescence-activated cell sorting (FACS) with pooled tetramers (FIG. 8C). TCRβ sequences from tetramer sorted cells were compared with the VATS library to determine if the VATS library contained HLA-A2 vaccine-specific sequences. Overall, tetramer+ sorted T cells were enriched for VATS sequences, representing 0.46% of all TCRβ clonotypes (3 of 658) compared with 0.09% of clonotypes (51 of 56,772) in the tetramer− T cell population (FIG. 8D). The frequencies of the HLA-A2 tetramer+ VATS were tracked over time in vaccinated mice from before vaccination through 9 months after exposure. Significant increases were observed in the frequencies of the tetramer+ VATS 2 weeks after vaccination before decreasing in subsequent time points (FIG. 8E). This is a clear representation of the antigen-specific response to the vaccine, from the expansion of virus-specific T cells during primary exposure to the induction of the low-frequency memory T cells that remain in circulation. Despite being present at low frequencies in the circulation (<1:50,000), by identifying the VATS library, it is possible to identify the virus-specific sequences from a very small volume of blood. Overall, these data confirm the computational identification of the virus-specific response and that virus-specific TCRβ sequences can be readily identified even at very low frequencies within the circulating memory T cell population over long periods of time.

TABLE 8
HLA-A2.1 Vaccinia-Specific Peptides

| Peptides | ORF | SEQ ID NO: |
|---|---|---|
| RLYDYFTRV | I1L₂₁₁₋₂₁₉ | 675 |
| ILDDNLYKV | G5R₁₈₋₂₆ | 676 |
| ALDEKLFLI | A23R₂₇₃₋₂₈₁ | 677 |
| GLFDFVNFV | A46R₁₄₂₋₁₅₀ | 678 |
| KVDDTFYYV | C7L₇₄₋₈₂ | 679 |
| RVYEALYYV | D12L₂₅₁₋₂₅₉ | 680 |
| KIDDMIEEV | C9L₆₀₂₋₆₁₀ | 681 |
| SLSNLDFRL | F11L₃₄₀₋₃₄₈ | 682 |
| ILSDENYLL | A6L₁₇₁₋₁₇₉ | 683 |

Previously published HLA-A2 restricted peptides and corresponding open reading frames

### Example 6: Discussion

In the experiments shown in Examples 1 to 5, high-throughput TCR repertoire analyses from a large cohort of mice (n=58) were used to identify and track TCR sequences responding to either the ACAM2000 smallpox vaccine or infection with MPXV. In total, >2.8×106 unique TCRβ clonotypes were analyzed from 245 individual blood samples collected before and after exposure. Data from mice administered the ACAM2000 smallpox vaccine were used to identify a library of 315 VATS. The VATS library acted as a diagnostic classifier, differentiating between naive and vaccinated or infected mice on the basis of the presence or absence of the public TCRβ sequences. The VATS library correctly identified samples from mice vaccinated with the smallpox vaccine and infected with MPXV from 2 weeks up to 9 months post-vaccination or infection. Overall, the diagnostic classifier was capable of distinguishing between vaccinated or infected samples and naive repertoires with >95% accuracy, which was replicated with MPXV-infected mice and the generation of the MATS library.

It was confirmed that the VATS library represented the public vaccine-specific T cell population by comparing the VATS library with TCRs expanded after in vitro culture with ACAM2000 and from vaccinia-specific HLA-A2.1 tetramer sorted T cells. The overlap between the TCR repertoires identified by in vitro expansion or tetramer sorting and the VATS library was limited. This was expected given the large number of immune-recognized Orthopoxvirus epitopes, various MHC molecules in mice, and that the majority of antigen-specific TCR clonotypes are private (specific to the individual mouse). This was previously shown in the human TCR repertoire, analyzing HLA-A2-restricted CD8+ T cells recognizing Epstein-Barr virus- and CMV-specific epitopes (Venturi et al., 2008a). Although only a small number of VATS sequences were found in the tetramer+ TCRβ repertoire, those sequences could be readily tracked and identified in mice up to 9 months after vaccination. Thus, this approach allowed the development of a tool to follow the virus-specific response over sequential time points in an aging population of mice. The frequencies at which the tetramer+ VATS were found in the circulation by TCR sequencing were as low as 1:50,000, meaning that using the VATS library to probe the TCRβ repertoire can be more sensitive than other technologies such as tetramer staining, intracellular cytokine staining, or allele-specific oligonucleotide PCR (Campana, 2010, Faham et al., 2012, Wolf and DiPaolo, 2016, van der Velden et al., 2014, van der Velden and van Dongen, 2009).

The low-frequency virus-specific memory response previously could only be readily measured through immune assays screening for serum antibodies against vaccinia and other pox viruses, which have historically been shown to be a very powerful and accurate tool for determining an individual's prior exposure to Orthopoxvirus (Frey et al., 2003, Newman et al., 2003, Yin et al., 2013). However, we have shown that immunosequencing of the TCRβ chain can differentiate between naive and vaccinated or infected individuals with approximately the same level of accuracy (Hammarlund et al., 2005). Considering the nature of the two methods, there are some key differences between immunosequencing of the TCR repertoire and serum antibody profiling. A major difference is that immunosequencing relies on genomic DNA or cDNA. In areas of the world where resources are limited, the molecular stability and shelf life of DNA compared with serum and antibodies offers significant benefit when collecting and transporting samples. Additionally, once the appropriate pathogen-associated TCR sequence libraries are developed, an individual's TCR repertoire could easily be used to determine prior exposure to a host of different pathogens with virtually no increase in effort, whereas testing serum for antibodies would require multiple tests for subsequent infectious agents (Emerson and DeWitt, 2017). Although the initial effort of collecting and analyzing large training cohorts for each target pathogen may be substantial, as sequencing data from subsequent studies become available, the resources required to produce large datasets becomes less (Emerson and DeWitt, 2017). Immunosequencing data, when published, are archived in public databases. This allows researchers to use previously published TCR repertoire data to increase diagnostic power while lowering the resources required to achieve large sample sizes (DeWitt et al., 2016).

Using this technology, it may be possible to develop panels of TCR sequence libraries capable of determining individuals' prior exposure to multiple pathogens simultaneously. The ability to discern an individual's immunological history has significant clinical benefits. Additionally, it is known that multiple viruses are able to undergo rapid mutation to evade immune detection. Analysis of the TCR repertoire could potentially be used in an attempt to track viral variants. However, we recognize that there are significant challenges involved in recapitulating this approach in human populations. Compared with genetically identical mice, humans display significant diversity in their HLA haplotypes, including rare HLAs, and TCR repertoires will likely limit the detection of public TCRs. Additionally, human populations are under constant exposure to different commensals, pathogens, and environmental stimuli, which can make identifying TCR sequences recognizing specific pathogens significantly more difficult. However, by acquiring larger blood volumes (5 mL in human versus 100 μL in mouse) and performing sequencing at greater depths (e.g., using ultra-deep sequencing), generating TCR repertoires of hundreds of thousands of clonotypes per sample, it is possible to perform similar studies in humans. This has been shown to be possible using populations of CMV+ and CMV− human populations (Emerson and DeWitt, 2017). Future studies are focused on adapting the current methodology to determine prior pathogen-specific exposure in human populations.

In summary, we have demonstrated that immunosequencing is a powerful and highly versatile tool for analyzing the TCR repertoire. In this study, an extensive database of TCRβ sequences was generated from the circulating TCR repertoires from a large cohort of mice (n=58) and used to identify and track the vaccine-specific T cell response over time. This allowed a comprehensive analysis of Orthopoxvirus-associated TCR sequences and shows that analyses of TCRβ repertoires can be used to determine individuals' prior exposure to ACAM2000 or MPXV with a high degree of accuracy and is capable of tracking the virus-specific populations present at ultra-low frequencies long after primary exposure resolved.

### Example 7: iCAT Diagnostic Assessment Tool of Immunology History Using High-Throughput TCR Sequencing

iCAT is a user-friendly, graphical-interface software that takes exposed and non-exposed samples of T-cell receptor (TCR) clonotypes as input and identifies pathogen-specific TCR sequences. Using these sequences, iCAT can also classify independent samples of TCR clonotypes. iCAT's backend methodology is based on performing Fisher's exact tests to find informative TCR sequences. When tested on mice samples from a recent publication, iCAT was able to identify vaccine-associated TCR sequences with 95% accuracy. With iCAT, we capitalize on the power of TCR sequencing to simplify infection diagnosis and further investigation of immunological history.

Software (iCAT) Framework

iCAT provides a graphical user interface in the form of a web-app by the power of R-Shiny. The user can upload multiple TCR sequence repertoires from negative (control) and positive (experimental) cohorts. iCAT accepts tab-delimited files with the size limit of 10 gigabytes per file. The user can select amongst three unique options to define TCR clonotypes within samples as well as parameters of the analyses: nucleotide sequences (“nucleotide”), CDR3 amino acid sequences (“aminoAcid”), or a combination of TCRV name, CDR3 sequence, and TCRJ name (“vGeneName”, “amino-Acid”, and “jGeneName”) where column name is represented in parentheses. Users can change an upper p-value threshold for performing Fisher Exact tests, which are used to identify TCR sequences of interest.

Clicking “Train” will start the pipeline to statistically identify a subset of target-associated sequences (TARSs) that give signal about the identity of the samples, negative or positive. As this is not typically an instantaneous process and can often be the bottleneck of analyses with large data, a graphical progress bar is implemented to provide status updates about the iCAT pipeline. Upon training, iCAT's main tab provides a table summary of the data, a figure shows the distribution of TARSs between the positive and negative samples, and a classification matrix shows the expected accuracy if those sequences were used for characterization, which is estimated from the training data.

A separate tab, “Library”, is unlocked upon training and shows a table where each row describes a TARS and its presence in the positive and negative samples. All tables and figures are supplemented with a custom button for easy download. The third tab of iCAT, “Prediction”, also unlocks after training and allows the user to upload one or more independent TCR-sequencing samples for classification. iCAT will provide a downloadable table with the predictions if more than one sample was uploaded.

### Diagnostic Classification

The statistical methodology of iCAT is based on identifying a subset of TARSs that informs classification. TCR sequences significantly associated with positive samples as opposed to negative samples are identified by performing Fisher's exact test. iCAT determines the optimal p-value cutoff based on the idea of coverage ratio describes above in Materials and Methods. Coverage was defined as the sum of samples containing each TARS divided by the total number of samples. iCAT calculates the coverage for negative samples (Cn) and positive samples (Cp). The coverage ratio, Cn:Cp, is calculated for each p-value. The optimal p-value is thus defined as the p-value with the maximum coverage ratio.

To classify an independent sample, iCAT first determines the percentage of TARSs in the sample. This percentage is compared to the normal distributions of negative and positive samples previously observed. The probability density function of the independent TARS percentage is calculated for the positive and negative normal distributions, with an internal normalization factor to reduce potential overfitting of the classifier. Classification is determined by the strength of samples' association with the positive or negative training data. Independent sample classification is used as a method to cross-validate the diagnostic accuracy of the classifier and for performing Leave-one-out analyses by removing all samples from a single source (mouse, individual, etc.) from the training data and re-training the classifier, using the removed samples in the prediction tab.

### Results

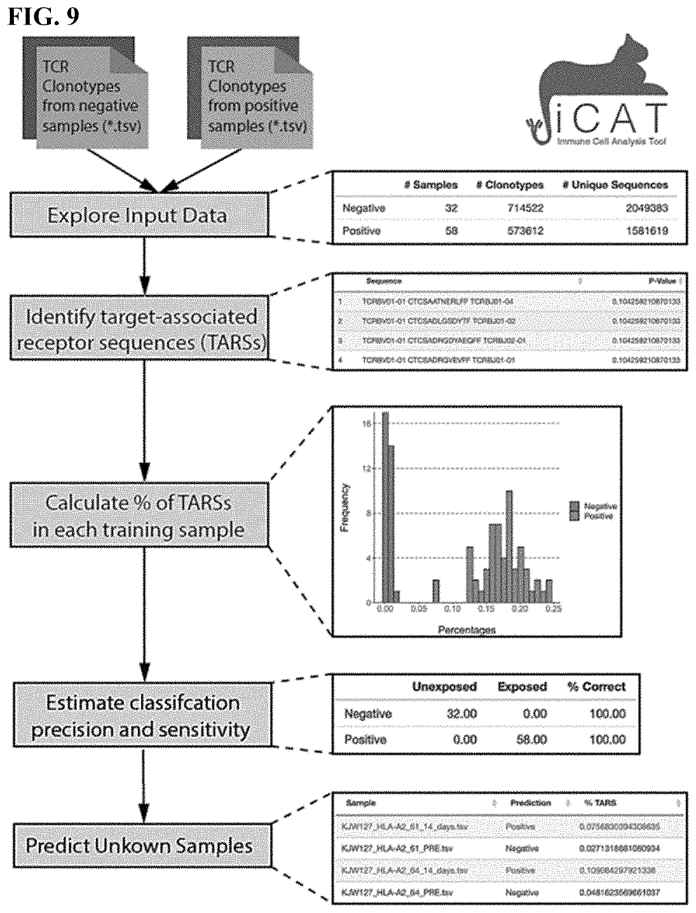

iCAT was used to identify vaccine-associated receptor sequences in mice injected with the smallpox vaccine (Wolf, et al., 2018). 32 pre-exposure (naïve) samples were analyzed, which included 714,522 clonotypes and 2,049,383 unique CDR3 amino acid sequences. 58 samples taken 2- and 8-weeks post-vaccination were analyzed, which included 573,612 and 1,581,619 unique CDR3 amino acid sequences (FIG. 9). iCAT accurately generated the same virus-associated TCR library (314 TCR sequences) identified in Wolf, et al. The library was used to train the diagnostic classifier.

From the training data, iCAT correctly classified 32 of 32 naïve samples as “unexposed” and 58 of 58 vaccinated samples as “exposed” (100% accuracy). TCR repertoires from 10 mice pre- and 2-weeks postvaccination were used as an independent cross-validation cohort and iCAT classified them with 95% accuracy. Overall, this data displays that the iCAT platform computationally identifies TCR sequences associated with exposure to a pathogen, training a diagnostic classifier to distinguish between exposed and unexposed samples with a high degree of accuracy. See FIG. 9.

### Example 7: Generation of a Human Vaccine Associated TCR Library

A human cohort was vaccinated with the ACAM2000 vaccine. T-cells were isolated and analyzed before and after the vaccine administration. As above, the genomic DNA of these T-cells were amplified and sequences to generate TCRβ clonotype profiles of the test subjects. The data generated was fed to a neural network program which trained on the data to identify unique TCRβ alleles statistically associated with small pox vaccination, using methods similar to those described above with changes as described below. The alleles identified are presented in Table 3 above. A detailed explanation is provided below.

### Summary and Results

During an infection or vaccination, T-cells that carry receptors specific to a certain pathogen become activated and each receptor is encoded by a uniquely rearranged DNA sequence. Even after the infection is eliminated, these activated immune cells remain and serve to prevent secondary infection of that pathogen. As a result of this persistence in the body, analyzing the large and diverse TCR repertoire may help us better understand immune system features and disease progression. In addition, the unique DNA rearrangements could be stable biomarkers for reliable diagnosis of infectious diseases. Recently, high-throughput NGS techniques were employed to analyze the diverse immune cell repertoire. A few research labs including us have attempted to develop statistical methods to identify and classify TCR sequences corresponding to a specific viral infection; however, it is challenging and critical to increase the accuracy of identifying viral infection from the diverse TCR repertoire over time and within the same individual. Especially, predicting human viral infection requires fast diagnosis and high accuracy. Satisfying both speed and accuracy requires much effort and skill.

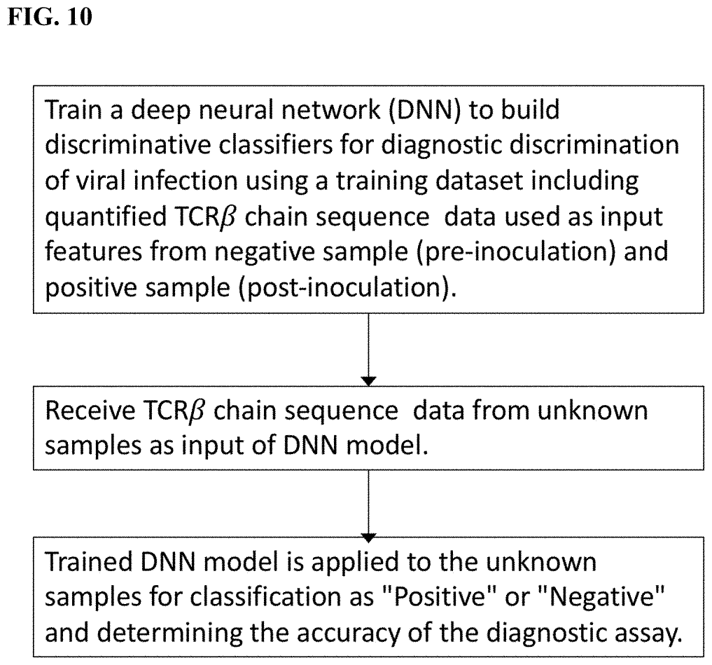

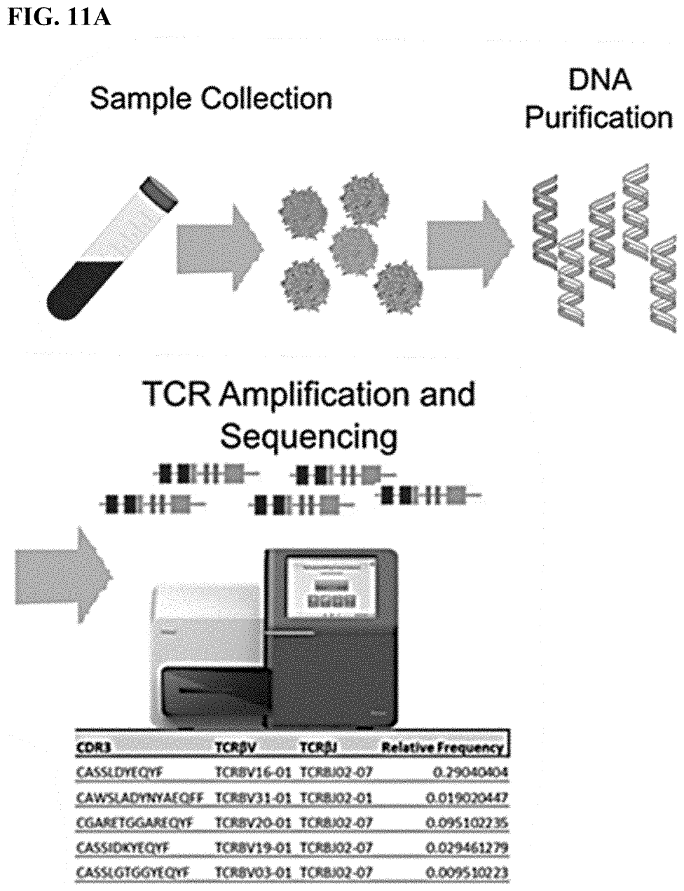

After the analysis of the mouse dataset, we tried to analyze much larger and complex samples from smallpox vaccinated human cohorts (FIG. 11A). The dataset is composed of a few hundreds of samples for pre- and post-exposure to smallpox vaccines from more than 100 volunteer cohorts where each sample has hundreds of millions of TCR sequences. We have investigated the statistical power of the iCAT to derive a subset of target-associated sequences (TARSs) and perform the prediction test from independent TCR-sequencing samples, but the accuracy of prediction is much lower (˜50%) than the mouse dataset. We expected a lower prediction accuracy because of the enormous amount of genetic diversity of human samples and HLA types, but the prediction rate that we achieved was lower than our expectation. To overcome these challenges, we applied advanced deep learning approach to increase the accuracy of prediction (FIG. 10, FIG. 11). Applying the deep neuronal network (DNN) model increased accuracy of independent test samples to 97% surprisingly (FIG. 11C).

In detail, we first preprocessed 129 samples were preprocessed to generate 2,525,775 unique TCR sequences and their frequency in each sample. This data (both the sequences and their frequency) was used as input features in a classifier to train it to identify pre- and post-vaccination of smallpox vaccine from 96 of the 129 samples. The remaining 33 samples were saved for a later independent test. Preliminary optimization results showed that using 5 hidden layers, 90 nodes (neurons), and 1000 max iterations (FIG. 11B) resulted in the highest (97%) prediction accuracy rate when tested on previously unseen (independent test) samples and retained its 100% accuracy when identifying previously seen (training) samples (FIG. 11C). The parameters were properly configured by randomized hyper-parameter search strategy since the DNN algorithm may affect proposed model's effectiveness. If inappropriate parameters are selected, the weights or coefficients in DNN do not converge, and the trained models are not usable.

### DNN Learning Algorithms

As one of the most powerful machine learning methods, the deep learning neural network has been substantially employed to explore the high-level features hidden in biomedical data. In this example, the deep learning framework was used to train the deep learning models for diagnostic discrimination. A multi-layer neural network (i.e., more than three layers) was used to extract hidden patterns from the input features through differing numbers of hidden layers. The extracted hidden features were finally fed into the last layer of logistic regression to classify the sample into binary classes. The deep learning model can be optimized through minimizing the binary cross-entropy objective function in the process of standard error backward propagation. Similar to the other three methods, the training dataset was equally split into five sets, and five-fold cross-validation was used to train and validate the model's robustness. Several parameters of neural networks were also adjusted using the repeated cross-validation, including the number of hidden layers, the number of hidden nodes in each hidden layer, and the types of activation functions for the hidden nodes. Several hyper-parameters were also tuned, including the dropout rate for regularization, learning rate and momentum used in different types of optimization algorithms. The classification accuracy is calculated for each round of five-fold cross-validation, and the accuracy scores are averaged over a total of 50 rounds to select the best parameter set for final testing. The deep neural network was implemented using the Tensorflow library (www.tensorflow.org), along with the cross-validation and parameter tuning available in the Scikit-learn library.

### Model Training

In this work, the predictive ability of the DNN method for diagnostic discrimination of viral infection was evaluated to understand how immune system features can diagnose viral infection status. The frequency counts of all CDR3 amino acid sequences (a.k.a. peptides) were calculated from quantified TCR beta chain sequence data and used as input features for machine learning methods to build discriminative classifiers. Each negative sample (pre-inoculation) or positive sample (post-inoculation) was described as a vector of frequency counts, each representing the number of CDR3 amino acids found in the sequence data of the sample.

The analysis started with the data partition. A stratified sampling method was applied to randomly divide the data into subsets according to the status of infection (pre- or post-introduction). Each of the three datasets were partitioned into a training set and testing set with a ratio of 75%/25%. The repeated 5-fold cross-validation was used to estimate the optimal parameters of each machine learning algorithm on the training dataset. The best training parameters selected by cross-validation were used to retrain the whole training dataset to derive the final model for evaluation. The independent testing subset is only seen when the final model of each algorithm is determined. After the training data was collected, several data normalization schemes were attempted before applying machine learning algorithms for model learning. Due to the different experimental conditions (i.e., sequence depth) and sample variations, the number of frequency counts for amino acids might vary in magnitude. Normalization might be necessary to remove inherent bias for different machine learning methods. To this end, the training data was transformed in three ways: (1) peptide-based normalization that normalizes counts across all training samples within each amino acid sequence; (2) sample-based normalization that normalizes counts of amino acids within individual samples; and (3) the benchmark data that uses original counts without any normalization. The Minimum-Maximum transformation was adopted to convert counts into the range between zero and one when the normalization is needed. The normalized/original features was then used to train different machine learning models for infection diagnosis.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above methods without departing from the scope of the invention, it is intended that all matter contained in the above description and shown in the accompanying drawings shall be interpreted as illustrative and not in a limiting sense.

### REFERENCES

Arstila, T. P., Casrouge, A., Baron, V., Even, J., Kanellopoulos, J., and Kourilsky, P. (1999). “A direct estimate of the human alphabeta T cell receptor diversity.” Science 286, 958-961.
Cabaniols, J. P., Fazilleau, N., Casrouge, A., Kourilsky, P., and Kanellopoulos, J. M. (2001). “Most alpha/beta T cell receptor diversity is due to terminal deoxynucleotidyl transferase.” J. Exp. Med. 194, 1385-1390.
Campana, D. (2010). “Minimal residual disease in acute lymphoblastic leukemia.” Hematology Am. Soc. Hematol. Educ. Program 2010, 7-12.
Carlson, C. S., Emerson, R. O., Sherwood, A. M., Desmarais, C., Chung, M. W., Parsons, J. M., Steen, M. S., LaMadrid-Herrmannsfeldt, M. A., Williamson, D. W., Livingston, R. J., et al. (2013). “Using synthetic templates to design an unbiased multiplex PCR assay.” Nat. Commun. 4, 2680.
Chen, W., Anto'n, L. C., Bennink, J. R., and Yewdell, J. W. (2000). “Dissecting the multifactorial causes of immunodominance in class I-restricted T cell responses to viruses.” Immunity 12, 83-93.
Davis, M. M., and Bjorkman, P. J. (1988). “T-cell antigen receptor genes and T-cell recognition.” Nature 334, 395-402.
DeWitt, W. S., Emerson, R. O., Lindau, P., Vignali, M., Snyder, T. M., Desmarais, C., Sanders, C., Utsugi, H., Warren, E. H., McElrath, J., et al. (2015). “Dynamics of the cytotoxic T cell response to a model of acute viral infection.” J. Virol. 89, 4517-4526.
DeWitt, W. S., Lindau, P., Snyder, T. M., Sherwood, A. M., Vignali, M., Carlson, C. S., Greenberg, P. D., Duerkopp, N., Emerson, R. O., and Robins, H. S. (2016). “A public database of memory and naive B-Cell receptor sequences.” PLoSONE 11, e0160853.
Douillard, P., Josien, R., Pannetier, C., Menoret, S., Kourilsky, P., Soulillou, J. P., and Cuturi, M. C. (1997). “TCR V beta repertoire in LEW.1W heart allografts acutely rejected by LEW.1A recipients is restricted and comprises a public response.” Transplant. Proc. 29, 1054.
Elhanati, Y., Murugan, A., Callan, C. G., Jr., Mora, T., and Walczak, A. M. (2014). “Quantifying selection in immune receptor repertoires.” Proc. Nat. Acad. Sci. USA 111, 9875-9880.
Emerson, R. O., and DeWitt, W. S. (2017). “Immunosequencing identifies signatures of cytomegalovirus exposure history and HLA-mediated effects on the T cell repertoire.” Nat. Genet. 49, 659-665.
Estorninho, M., Gibson, V. B., Kronenberg-Versteeg, D., Liu, Y. F., Ni, C., Cerosaletti, K., and Peakman, M. (2013). “A novel approach to tracking antigen experienced CD4 T cells into functional compartments via tandem deep and shallow TCR clonotyping.” J. Immunol. 191, 5430-5440.
Faham, M., Zheng, J., Moorhead, M., Carlton, V. E., Stow, P., Coustan-Smith, E., Pui, C. H., and Campana, D. (2012). “Deep-sequencing approach for minimal residual disease detection in acute lymphoblastic leukemia.” Blood 120, 5173-5180.
Frey, S. E., Newman, F. K., Cruz, J., Shelton, W. B., Tennant, J. M., Polach, T., Rothman, A. L., Kennedy, J. S., Wolff, M., Belshe, R. B., and Ennis, F. A. (2002). “Dose-related effects of smallpox vaccine.” N. Engl. J. Med. 346, 1275-1280.
Frey, S. E., Newman, F. K., Yan, L., Lottenbach, K. R., and Belshe, R. B. (2003). “Response to smallpox vaccine in persons immunized in the distant past.” JAMA 289, 3295-3299.
Gilchuk, P., Spencer, C. T., Conant, S. B., Hill, T., Gray, J. J., Niu, X., Zheng, M., Erickson, J. J., Boyd, K. L., McAfee, K. J., et al. (2013). “Discovering naturally processed antigenic determinants that confer protective T cell immunity.” J. Clin. Invest. 123, 1976-1987.
Hammarlund, E., Lewis, M. W., Carter, S. V., Amanna, I., Hansen, S. G., Strelow, L. I., Wong, S. W., Yoshihara, P., Hanifin, J. M., and Slifka, M. K. (2005). “Multiple diagnostic techniques identify previously vaccinated individuals with protective immunity against monkeypox.” Nat. Med. 11, 1005-1011.
Hancock, G., Yang, H., Yorke, E., Wainwright, E., Bourne, V., Frisbee, A., Payne, T. L., Berrong, M., Ferrari, G., Chopera, D., et al. (2015). “Identification of effective subdominant anti-HIV-1 CD8+ T cells within entire post-infection and post-vaccination immune responses.” PLoS Pathog. 11, e1004658.
Handley, L., Buller, R. M., Frey, S. E., Bellone, C., and Parker, S. (2009). “The new ACAM2000 vaccine and other therapies to control orthopoxvirus outbreaks and bioterror attacks.” Expert Rev. Vaccines 8, 841-850.
Heit, A., Schmitz, F., Gerdts, S., Flach, B., Moore, M. S., and Perkins, J. A. (2017). “Vaccination establishes clonal relatives of germinal center T cells in the blood of humans.” J. Exp. Med. 214, 2139-2152.
Ishizuka, J., Grebe, K., Shenderov, E., Peters, B., Chen, Q., Peng, Y., Wang, L., Dong, T., Pasquetto, V., Oseroff, C., et al. (2009). “Quantitating T cell crossreactivity for unrelated peptide antigens.” J. Immunol. 183, 4337-4345.
Kim, Y., Yewdell, J. W., Sette, A., and Peters, B. (2013). “Positional bias of MHC class I restricted T-cell epitopes in viral antigens is likely due to a bias in conservation.” PLoS Comput. Biol. 9, e1002884.
Kirsch, I., Vignali, M., and Robins, H. (2015). “T-cell receptor profiling in cancer.” Mol. Oncol. 9, 2063-2070.
Kotturi, M. F., Assarsson, E., Peters, B., Grey, H., Oseroff, C., Pasquetto, V., and Sette, A. (2009). “Of mice and humans: how good are HLA transgenic mice as a model of human immune responses?” Immunome Res. 5, 3.
Li, H., Ye, C., Ji, G., and Han, J. (2012). “Determinants of public T cell responses.” Cell Res. 22, 33-42.
Lim, A., Trautmann, L., Peyrat, M. A., Couedel, C., Davodeau, F., Romagne', F., Kourilsky, P., and Bonneville, M. (2000). “Frequent contribution of T cell clonotypes with public TCR features to the chronic response against a dominant EBV-derived epitope: application to direct detection of their molecular imprint on the human peripheral T cell repertoire.” J. Immunol. 165, 2001-2011.
Logan, A. C., Vashi, N., Faham, M., Carlton, V., Kong, K., Buno, I., Zheng, J., Moorhead, M., Klinger, M., Zhang, B., et al. (2014). “Immunoglobulin and T cell receptor gene high-throughput sequencing quantifies minimal residual disease in acute lymphoblastic leukemia and predicts post-transplantation relapse and survival.” Biol. Blood Marrow Transplant. 20, 1307-1313.
Lossius, A., Johansen, J. N., Vartdal, F., Robins, H., Jürate, Šaltyté, B., Holmøy, T., and Olweus, J. (2014). “High-throughput sequencing of TCR repertoires in multiple sclerosis reveals intrathecal enrichment of EBV-reactive CD8+ T cells.” Eur. J Immunol. 44, 3439-3452.
Manfras, B. J., Terjung, D., and Boehm, B. O. (1999). “Non-productive human TCR beta chain genes represent V-D-J diversity before selection upon function: insight into biased usage of TCRBD and TCRBJ genes and diversity of CDR3 region length”. Hum. Immunol. 60, 1090-1100.
Marrero, I., Aguilera, C., Hamm, D. E., Quinn, A., and Kumar, V. (2016). “High throughput sequencing reveals restricted TCR V usage and public TCRO clonotypes among pancreatic lymph node memory CD4(+) T cells and their involvement in autoimmune diabetes.” Mol. Immunol. 74, 82-95.
Miller, J. D., van der Most, R. G., Akondy, R. S., Glidewell, J. T., Albott, S., Masopust, D., Murali-Krishna, K., Mahar, P. L., Edupuganti, S., Lalor, S., et al. (2008). “Human effector and memory CD8+ T cell responses to smallpox and yellow fever vaccines.” Immunity 28, 710-722.
Moutaftsi, M., Salek-Ardakani, S., Croft, M., Peters, B., Sidney, J., Grey, H., and Sette, A. (2009). “Correlates of protection efficacy induced by vaccinia virus-specific CD8+ T-cell epitopes in the murine intranasal challenge model.” Eur. J. Immunol. 39, 717-722.
Newman, F. K., Frey, S. E., Blevins, T. P., Mandava, M., Bonifacio, A., Jr., Yan, L., and Belshe, R. B. (2003). “Improved assay to detect neutralizing antibody following vaccination with diluted or undiluted vaccinia (Dryvax) vaccine.” J. Clin. Microbiol. 41, 3154-3157.
Parker, S., and Buller, R. M. (2013). “A review of experimental and natural infections of animals with monkeypox virus between 1958 and 2012.” Future Virol. 8, 129-157.
Parker, S., Crump, R., Foster, S., Hartzler, H., Hembrador, E., Lanier, E. R., Painter, G., Schriewer, J., Trost, L. C., and Buller, R. M. (2014). “Co-administration of the broad-spectrum antiviral, brincidofovir (CMX001), with smallpox vaccine does not compromise vaccine protection in mice challenged with ectromelia virus.” Antiviral Res. 111, 42-52.
Robins, H. S., Campregher, P. V., Srivastava, S. K., Wacher, A., Turtle, C. J., Kahsai, O., Riddell, S. R., Warren, E. H., and Carlson, C. S. (2009). “Comprehensive assessment of T-cell receptor beta-chain diversity in alphabeta T cells.” Blood 114, 4099-4107.
Robins, H. S., Srivastava, S. K., Campregher, P. V., Turtle, C. J., Andriesen, J., Riddell, S. R., Carlson, C. S., and Warren, E. H. (2010). “Overlap and effective size of the human CD8+ T cell receptor repertoire.” Sci. Transl. Med. 2, 47ra64.
Shchelkunov, S. N., Totmenin, A. V., Safronov, P. F., Mikheev, M. V., Gutorov, V. V., Ryazankina, O. I., Petrov, N. A., Babkin, I. V., Uvarova, E. A., Sandakhchiev, L. S., et al. (2002). “Analysis of the monkeypox virus genome.” Virology 297, 172-194.
Stabenow, J., Buller, R. M., Schriewer, J., West, C., Sagartz, J. E., and Parker, S. (2010). “A mouse model of lethal infection for evaluating prophylactics and therapeutics against Monkeypox virus.” J. Virol. 84, 3909-3920.
Thomas, N., Best, K., Cinelli, M., Reich-Zeliger, S., Gal, H., Shifrut, E., Madi, A., Friedman, N., Shawe-Taylor, J., and Chain, B. (2014). “Tracking global changes induced in the CD4 T-cell receptor repertoire by immunization with a complex antigen using short stretches of CDR3 protein sequence.” Bioinformatics 30, 3181-3188.
Valkenburg, S. A., Josephs, T. M., Clemens, E. B., Grant, E. J., Nguyen, T. H., Wang, G. C., Price, D. A., Miller, A., Tong, S. Y., Thomas, P. G., et al. (2016). “Molecular basis for universal HLA-A*0201-restricted CD8+ T-cell immunity against influenza viruses.” Proc. Natl. Acad. Sci. USA 113, 4440-4445.
van der Velden, V. H., and van Dongen, J. J. (2009). “MRD detection in acute lymphoblastic leukemia patients using Ig/TCR gene rearrangements as targets for real-time quantitative PCR.” Methods Mol. Biol. 538, 115-150.
van der Velden, V. H., Noordijk, R., Brussee, M., Hoogeveen, P. G., Homburg, C., de Haas, V., van der Schoot, C. E., and van Dongen, J. J. (2014). “Minimal residual disease diagnostics in acute lymphoblastic leukaemia: impact of primer characteristics and size of junctional regions.” Br. J. Haematol. 164, 451-453.
Venturi, V., Chin, H. Y., Asher, T. E., Ladell, K., Scheinberg, P., Bornstein, E., van Bockel, D., Kelleher, A. D., Douek, D. C., Price, D. A., and Davenport, M. P. (2008a). “TCR beta-chain sharing in human CD8+ T cell responses to cytomegalovirus and EBV.” J. Immunol. 181, 7853-7862.
Venturi, V., Price, D. A., Douek, D. C., and Davenport, M. P. (2008b). “The molecular basis for public T-cell responses?” Nat. Rev. Immunol. 8, 231-238.
Venturi, V., Nzingha, K., and Amos, T. G. (2016). “The neonatal CD8+ T cell repertoire rapidly diversifies during persistent viral infection.” J. Immunol. 196, 1604-1616.
Wolf, K., and DiPaolo, R. (2016). Immunosequencing: accelerating discovery in immunology and medicine. Curr. Trends Immunol. 17, 85-93.
Wolf, K. J., Emerson, R. O., Pingel, J., Buller, R. M., and DiPaolo, R. J. (2016). “Conventional and regulatory CD4+ T cells that share identical TCRs are derived from common clones.” PLoS ONE 11, e0153705.
Yin, L., Calvo-Calle, J. M., Cruz, J., Newman, F. K., Frey, S. E., Ennis, F. A., and Stern, L. J. (2013). “CD4+ T cells provide intermolecular help to generate robust antibody responses in vaccinia virus-vaccinated humans.” J. Immunol. 190, 6023-6033.
Yousfi Monod, M., Giudicelli, V., Chaume, D., and Lefranc, M. P. (2004). “IMGT/JunctionAnalysis: the first tool for the analysis of the immunoglobulin and T cell receptor complex V-J and V-D-J JUNCTIONs.” Bioinformatics 20 (Suppl 1), i379-i385.

1. A method for determining whether a subject has been exposed to an immunogenic antigen, the method comprising:
a. amplifying and sequencing TCRβ alleles in mRNA and/or genomic DNA obtained from T-cells of the subject;
b. identifying unique TCRβ alleles sequences in T-cells of the subject to generate a TCRβ clonotype profile of the subject;
c. comparing the TCRβ clonotype profile of the subject to a database of target associated receptor sequences (TARSs) comprising unique TCRβ alleles identified as associated with exposure to the immunogenic antigen in a cohort of independent test subjects;
d. generating a diagnostic classifier of the subject comprising the number of TARSs identified in the subject relative to the total number of unique TCRβ alleles in the subject; and
e. determining that the subject has been exposed to the immunogenic antigen if the diagnostic classifier exceeds a predetermined threshold for the diagnostic classifier, wherein the predetermined threshold is determined by the prevalence of TARSs in the test cohort after exposure to the immunogenic antigen.

2. The method of claim 1 wherein the generation of the database of “target associated receptor sequences” (TARSs) comprises:
a. amplifying and sequencing TCRβ alleles in mRNA and/or genomic DNA obtained from T cells of the test subjects, wherein the T cells are isolated before and after exposure to the immunogenic antigen;
b. identifying unique TCRβ allele sequences in the cohort of test subject;
c. performing a Fisher exact test on each unique TCRβ sequence to generate a statistical association between the TCRβ sequence and the exposure status of the subject; and
d. generating the database of TARSs comprising unique TCRβ sequences having a p-value that exceeds a p-value threshold.

3. The method of claim 2 wherein the p-value threshold is the p-value that generates a TARSs database having the maximum coverage ratio defined as the ratio of Cp to Cn, wherein Cp and Cn are, respectively, the proportion of exposed (Cp) or naïve (Cn) samples having at least one TCRβ sequence included in the TARSs database relative to the total number of exposed samples (Cp) or naïve samples (Cn).

4. The method of claim 1 wherein determining that the subject has been exposed to the antigen further comprises applying a probability distribution function comparing the diagnostic classifier of the subject to a distribution of TARSs prevalence in the test subject cohort after exposure to the immunogenic antigen.

5. The method of claim 1 further comprising dynamically tracking an immune response of the subject over time, the method comprising generating a plurality of diagnostic classifier scores of the subject at different time points and comparing to a TARs database associated with the immune response; wherein generating the diagnostic classifier scores does not alter the TARSs database.

6. The method of claim 1 wherein the method comprises analyzing a sample of T-cells obtained from the subject up to 9 months after a potential exposure event to the immunogenic antigen.

7. (canceled)

8. The method of claim 1 wherein the database of TARSs is validated by identifying one or more splenocytes present in the test subjects of the cohort after exposure to the immunogenic antigen that express one or more of the TARSs.

9. The method of claim 8 wherein the splenocytes expressing one or more of the TARSs are identified by in vitro clonal expansion in response to treatment with the immunogenic antigen.

10. The method of claim 8 wherein the splenocytes expressing one or more of the TARSs are identified by a flow cytometry method wherein the splenocytes are isolated using a major histocompatibility complex (MHC) and antigenic peptide tetramers that are related to the immunogenic antigen.

11. The method of claim 1 wherein the TCRβ allele comprises the CDR3 variable region of a recombined TCRβ allele.

12. The method of claim 11, wherein an amino acid sequence encoded by the CDR3 variable region comprises any one of SEQ ID NOs: 1-674.

13. The method of claim 1 wherein the TCRβ allele comprises the V region, the CDR variable region and the J region of a recombined TCRβ allele.

14. The method claim 1 wherein the immunogenic antigen comprises a pathogen, an allergen, a vaccine, a virus or any immunogenic component or fragment thereof.

15. The method of claim 1 wherein the immunogenic antigen comprises a coronavirus, an influenza virus, an orthopoxvirus or any immunogenic component or fragment thereof.

16. (canceled)

17. The method of claim 16, wherein the immunogenic antigen comprises a SARS-CoV-2 virus.

18. (canceled)

19. (canceled)

20. The method of claim 1 wherein the immunogenic antigen comprises an orthopoxvirus vaccine, an influenza vaccine or a coronavirus vaccine.

21. (canceled)

22. (canceled)

23. (canceled)

24. (canceled)

25. (canceled)

26. A method of testing the efficacy of a vaccine, the method comprising:
a. Amplifying and sequencing TCRβ alleles in mRNA and/or genomic DNA of T-cells obtained from a subject after administration of the vaccine;
b. Comparing the TCRβ clonotype profile of the subject to a database of vaccine associated TCRβ sequences (VATSs) statistically associated with vaccination to generate a diagnostic classifier of the subject, wherein the diagnostic classifier comprises the number of VATSs identified in the subject relative to the total number of unique TCRβ alleles in the subject;
c. Determining that the vaccine is effective in generating an immune response if the diagnostic classifier exceeds a threshold determined by the prevalence of VATSs in an independent test cohort after exposure to the vaccine.

27. (canceled)

28. (canceled)

29. (canceled)

30. A method of identifying a viral infection in a subject, the method comprising:
a. amplifying and sequencing TCRβ alleles in mRNA and/or genomic DNA of T-cells obtained from the subject;
b. comparing the TCRβ sequences in the subject to one or more databases of virus-associated TCRβ sequences, wherein each database comprises TCRβ sequences statistically associated with one virus and each database is generated according to the method of claim 2; and
c. identifying the viral infection of the subject by determining the strength of the association of the TCRβ allele sequences identified in the subject to one or more of the databases.

31. A method of identifying an immune response in a subject, the method comprising identifying in the subject the presence of a significant number of unique TCR clonotypes that match a database of TCRβ sequences previously associated with the immune response in an independent cohort.

32. (canceled)

33. (canceled)

34. A method of generating a TCRβ database comprising TCRβ sequences statistically associated with an immune condition, exposure to a vaccine or immunogenic agent, and/or a pathogen, the method comprising:
a. amplifying and sequencing TCRβ alleles in mRNA and/or genomic DNA of T-cells obtained from a cohort of subjects having the immune condition, or having been exposed to the vaccine, immunogenic agent and/or pathogen; and
b. using a machine learning and/or neural network system to analyze the TCRβ allele sequences and statistically associate a subset of the TCRβ sequences to the immune condition, vaccine, immunogenic agent and/or pathogen.

35.-39. (canceled)


SEQUENCE LISTING (683 sequences)

SEQ ID NO: 1 - protein, 15 aa, Mus musculus
CASSDPGLGDYEQYF

SEQ ID NO: 2 - protein, 15 aa, Mus musculus
CASSSTGYNNQAPLF

SEQ ID NO: 3 - protein, 15 aa, Mus musculus
CTCSAEGGANTEVFF

SEQ ID NO: 4 - protein, 15 aa, Mus musculus
CASSLGLGNYAEQFF

SEQ ID NO: 5 - protein, 14 aa, Mus musculus
CASSLTGGNTEVFF

SEQ ID NO: 6 - protein, 14 aa, Mus musculus
CASSPRDREDTQYF

SEQ ID NO: 7 - protein, 12 aa, Mus musculus
CASSPDRDEQYF

SEQ ID NO: 8 - protein, 15 aa, Mus musculus
CASSQDGANTGQLYF

SEQ ID NO: 9 - protein, 13 aa, Mus musculus
CASSLEQNQAPLF

SEQ ID NO: 10 - protein, 13 aa, Mus musculus
CASSPTGNTEVFF

SEQ ID NO: 11 - protein, 13 aa, Mus musculus
CASSRSYNSPLYF

SEQ ID NO: 12 - protein, 11 aa, Mus musculus
CASSPGTEVFF

SEQ ID NO: 13 - protein, 12 aa, Mus musculus
CASSQDITEVFF

SEQ ID NO: 14 - protein, 15 aa, Mus musculus
CASSQDWVNYAEQFF

SEQ ID NO: 15 - protein, 11 aa, Mus musculus
CASSLGETLYF

SEQ ID NO: 16 - protein, 12 aa, Mus musculus
CASSDAGEEQYF

SEQ ID NO: 17 - protein, 13 aa, Mus musculus
CASGAGGEDTQYF

SEQ ID NO: 18 - protein, 14 aa, Mus musculus
CASGDTGAGNTLYF

SEQ ID NO: 19 - protein, 14 aa, Mus musculus
CASGEGLGKDTQYF

SEQ ID NO: 20 - protein, 14 aa, Mus musculus
CASGPTFNQDTQYF

SEQ ID NO: 21 - protein, 15 aa, Mus musculus
CASSFTGGNNQAPLF

SEQ ID NO: 22 - protein, 13 aa, Mus musculus
CASSLAGNERLFF

SEQ ID NO: 23 - protein, 16 aa, Mus musculus
CASSIGTGGNTGQLYF

SEQ ID NO: 24 - protein, 14 aa, Mus musculus
CASSLRGTGNTLYF

SEQ ID NO: 25 - protein, 15 aa, Mus musculus
CASSLTGGSNERLFF

SEQ ID NO: 26 - protein, 13 aa, Mus musculus
CASSLRDIYEQYF

SEQ ID NO: 27 - protein, 14 aa, Mus musculus
CAWSLDRYNSPLYF

SEQ ID NO: 28 - protein, 14 aa, Mus musculus
CAWSLPNSGNTLYF

SEQ ID NO: 29 - protein, 16 aa, Mus musculus
CTCSAAGTGVGNTLYF

SEQ ID NO: 30 - protein, 14 aa, Mus musculus
CTCSADRGSYEQYF

SEQ ID NO: 31 - protein, 16 aa, Mus musculus
CTCSAEDWGNYAEQFF

SEQ ID NO: 32 - protein, 14 aa, Mus musculus
CTCSAGGSNTEVFF

SEQ ID NO: 33 - protein, 13 aa, Mus musculus
CTCSAGRNSPLYF

SEQ ID NO: 34 - protein, 15 aa, Mus musculus
CTCSARTGGAGEQYF

SEQ ID NO: 35 - protein, 13 aa, Mus musculus
CASSQDGRGEQYF

SEQ ID NO: 36 - protein, 15 aa, Mus musculus
CASSQDRTGNTEVFF

SEQ ID NO: 37 - protein, 13 aa, Mus musculus
CASSQGGGTEVFF

SEQ ID NO: 38 - protein, 13 aa, Mus musculus
CASSFQANTEVFF

SEQ ID NO: 39 - protein, 13 aa, Mus musculus
CASSLARGYEQYF

SEQ ID NO: 40 - protein, 14 aa, Mus musculus
CASSLDSSNTEVFF

SEQ ID NO: 41 - protein, 15 aa, Mus musculus
CASSLGQGGGNTLYF

SEQ ID NO: 42 - protein, 13 aa, Mus musculus
CASSLKGQDTQYF

SEQ ID NO: 43 - protein, 13 aa, Mus musculus
CASSLSANTEVFF

SEQ ID NO: 44 - protein, 14 aa, Mus musculus
CASSQTGGAREQYF

SEQ ID NO: 45 - protein, 12 aa, Mus musculus
CASSYRNTEVFF

SEQ ID NO: 46 - protein, 13 aa, Mus musculus
CASRTISNERLFF

SEQ ID NO: 47 - protein, 12 aa, Mus musculus
CASSFDRGEVFF

SEQ ID NO: 48 - protein, 16 aa, Mus musculus
CASSPDWGGNTGQLYF

SEQ ID NO: 49 - protein, 15 aa, Mus musculus
CASSPLGVNQDTQYF

SEQ ID NO: 50 - protein, 12 aa, Mus musculus
CASSPTAYEQYF

SEQ ID NO: 51 - protein, 15 aa, Mus musculus
CASSQEGQGGDTQYF

SEQ ID NO: 52 - protein, 15 aa, Mus musculus
CASSQGDSSAETLYF

SEQ ID NO: 53 - protein, 14 aa, Mus musculus
CASSQGLSNERLFF

SEQ ID NO: 54 - protein, 15 aa, Mus musculus
CASSQLGGNTGQLYF

SEQ ID NO: 55 - protein, 13 aa, Mus musculus
CASSGQSNERLFF

SEQ ID NO: 56 - protein, 15 aa, Mus musculus
CASSLAGGGQNTLYF

SEQ ID NO: 57 - protein, 13 aa, Mus musculus
CASSLPTNSDYTF

SEQ ID NO: 58 - protein, 13 aa, Mus musculus
CASSLTGDYEQYF

SEQ ID NO: 59 - protein, 13 aa, Mus musculus
CASSLTNQDTQYF

SEQ ID NO: 60 - protein, 15 aa, Mus musculus
CASSWDWGSQNTLYF

SEQ ID NO: 61 - protein, 15 aa, Mus musculus
CASSLEGGSSYEQYF

SEQ ID NO: 62 - protein, 15 aa, Mus musculus
CASSLGLGVYAEQFF

SEQ ID NO: 63 - protein, 12 aa, Mus musculus
CASSLRGNTLYF

SEQ ID NO: 64 - protein, 13 aa, Mus musculus
CASSPDSGNTLYF

SEQ ID NO: 65 - protein, 13 aa, Mus musculus
CASSPGQGSDYTF

SEQ ID NO: 66 - protein, 14 aa, Mus musculus
CASRLGANTGQLYF

SEQ ID NO: 67 - protein, 15 aa, Mus musculus
CASSDAGLGFYEQYF

SEQ ID NO: 68 - protein, 14 aa, Mus musculus
CASSDAYSGNTLYF

SEQ ID NO: 69 - protein, 15 aa, Mus musculus
CASSDPGLGFYEQYF

SEQ ID NO: 70 - protein, 14 aa, Mus musculus
CASSDSANTGQLYF

SEQ ID NO: 71 - protein, 14 aa, Mus musculus
CASSETGNYAEQFF

SEQ ID NO: 72 - protein, 13 aa, Mus musculus
CASGAGAGNTLYF

SEQ ID NO: 73 - protein, 14 aa, Mus musculus
CASGDAGEQDTQYF

SEQ ID NO: 74 - protein, 14 aa, Mus musculus
CASGDARGENTLYF

SEQ ID NO: 75 - protein, 12 aa, Mus musculus
CASGDFNSPLYF

SEQ ID NO: 76 - protein, 13 aa, Mus musculus
CASGDRFSYEQYF

SEQ ID NO: 77 - protein, 14 aa, Mus musculus
CASGEAGDYAEQFF

SEQ ID NO: 78 - protein, 14 aa, Mus musculus
CASGPGQSNTEVFF

SEQ ID NO: 79 - protein, 14 aa, Mus musculus
CASSDAGSNERLFF

SEQ ID NO: 80 - protein, 14 aa, Mus musculus
CASSDATGGYEQYF

SEQ ID NO: 81 - protein, 14 aa, Mus musculus
CASSGTGVSYEQYF

SEQ ID NO: 82 - protein, 15 aa, Mus musculus
CASSFTGQNNQAPLF

SEQ ID NO: 83 - protein, 15 aa, Mus musculus
CASSFTGRNNQAPLF

SEQ ID NO: 84 - protein, 14 aa, Mus musculus
CASSLDKNTGQLYF

SEQ ID NO: 85 - protein, 12 aa, Mus musculus
CASSLGVYEQYF

SEQ ID NO: 86 - protein, 14 aa, Mus musculus
CASSLRGSGNTLYF

SEQ ID NO: 87 - protein, 13 aa, Mus musculus
CASSPGQYAEQFF

SEQ ID NO: 88 - protein, 14 aa, Mus musculus
CASSWGGNQDTQYF

SEQ ID NO: 89 - protein, 12 aa, Mus musculus
CASSRRQYEQYF

SEQ ID NO: 90 - protein, 15 aa, Mus musculus
CASSIRDWGGAEQFF

SEQ ID NO: 91 - protein, 14 aa, Mus musculus
CASSLTGNNQAPLF

SEQ ID NO: 92 - protein, 15 aa, Mus musculus
CASSMTGGSQNTLYF

SEQ ID NO: 93 - protein, 13 aa, Mus musculus
CASSRDKQDTQYF

SEQ ID NO: 94 - protein, 13 aa, Mus musculus
CGARDRGKNTLYF

SEQ ID NO: 95 - protein, 13 aa, Mus musculus
CGARVGSAETLYF

SEQ ID NO: 96 - protein, 13 aa, Mus musculus
CSSSQTNTGQLYF

SEQ ID NO: 97 - protein, 13 aa, Mus musculus
CASSLQKNTEVFF

SEQ ID NO: 98 - protein, 14 aa, Mus musculus
CASSLSRANSDYTF

SEQ ID NO: 99 - protein, 14 aa, Mus musculus
CASSLYRAGNTLYF

SEQ ID NO: 100 - protein, 14 aa, Mus musculus
CASSQDSYNSPLYF

SEQ ID NO: 101 - protein, 14 aa, Mus musculus
CASSRGVSGNTLYF

SEQ ID NO: 102 - protein, 14 aa, Mus musculus
CASSFGQGNTEVFF

SEQ ID NO: 103 - protein, 13 aa, Mus musculus
CASSFGSNERLFF

SEQ ID NO: 104 - protein, 14 aa, Mus musculus
CASSLGDSNERLFF

SEQ ID NO: 105 - protein, 14 aa, Mus musculus
CASSLGTGYAEQFF

SEQ ID NO: 106 - protein, 15 aa, Mus musculus
CASSLRDRNTGQLYF

SEQ ID NO: 107 - protein, 14 aa, Mus musculus
CASSRQGANSDYTF

SEQ ID NO: 108 - protein, 15 aa, Mus musculus
CASSSGTGSNERLFF

SEQ ID NO: 109 - protein, 11 aa, Mus musculus
CASSTGTEVFF

SEQ ID NO: 110 - protein, 13 aa, Mus musculus
CAWKGQSNSDYTF

SEQ ID NO: 111 - protein, 13 aa, Mus musculus
CAWSLEGRDTQYF

SEQ ID NO: 112 - protein, 11 aa, Mus musculus
CAWSPRDTQYF

SEQ ID NO: 113 - protein, 13 aa, Mus musculus
CAWSQGGNSDYTF

SEQ ID NO: 114 - protein, 14 aa, Mus musculus
CASSPGISNERLFF

SEQ ID NO: 115 - protein, 13 aa, Mus musculus
CASSQGGNSDYTF

SEQ ID NO: 116 - protein, 16 aa, Mus musculus
CASSQEGGVNQDTQYF

SEQ ID NO: 117 - protein, 13 aa, Mus musculus
CAWSLGGVYEQYF

SEQ ID NO: 118 - protein, 13 aa, Mus musculus
CAWSLQANTEVFF

SEQ ID NO: 119 - protein, 13 aa, Mus musculus
CASSRDSQNTLYF

SEQ ID NO: 120 - protein, 14 aa, Mus musculus
CASSLEGGNTEVFF

SEQ ID NO: 121 - protein, 12 aa, Mus musculus
CASSLGFYEQYF

SEQ ID NO: 122 - protein, 13 aa, Mus musculus
CASSRDKQDTQYF

SEQ ID NO: 123 - protein, 15 aa, Mus musculus
CASSSTGYNNQAPLF

SEQ ID NO: 124 - protein, 15 aa, Mus musculus
CTCSAEGVSNERLFF

SEQ ID NO: 125 - protein, 15 aa, Mus musculus
CASSFTGQNNQAPLF

SEQ ID NO: 126 - protein, 14 aa, Mus musculus
CASSRQGGDERLFF

SEQ ID NO: 127 - protein, 10 aa, Mus musculus
CASGNTEVFF

SEQ ID NO: 128 - protein, 12 aa, Mus musculus
CASSDAGAEQFF

SEQ ID NO: 129 - protein, 15 aa, Mus musculus
CASSFTGRNNQAPLF

SEQ ID NO: 130 - protein, 13 aa, Mus musculus
CASSRDRYAEQFF

SEQ ID NO: 131 - protein, 16 aa, Mus musculus
CTCSADLGTSAETLYF

SEQ ID NO: 132 - protein, 14 aa, Mus musculus
CASSPTTSAETLYF

SEQ ID NO: 133 - protein, 14 aa, Mus musculus
CASSHRDGQDTQYF

SEQ ID NO: 134 - protein, 12 aa, Mus musculus
CASGEGLGEQYF

SEQ ID NO: 135 - protein, 14 aa, Mus musculus
CASSQDRQGYEQYF

SEQ ID NO: 136 - protein, 14 aa, Mus musculus
CASSSDRHQDTQYF

SEQ ID NO: 137 - protein, 14 aa, Mus musculus
CASSQDLGPYEQYF

SEQ ID NO: 138 - protein, 11 aa, Mus musculus
CASSIRAEQYF

SEQ ID NO: 139 - protein, 15 aa, Mus musculus
CASSLTGGSSYEQYF

SEQ ID NO: 140 - protein, 16 aa, Mus musculus
CTCSAAGTGVGNTLYF

SEQ ID NO: 141 - protein, 12 aa, Mus musculus
CASSLTAYEQYF

SEQ ID NO: 142 - protein, 15 aa, Mus musculus
CASSQEGLGGREQYF

SEQ ID NO: 143 - protein, 14 aa, Mus musculus
CASSDPGGNERLFF

SEQ ID NO: 144 - protein, 15 aa, Mus musculus
CASSQEGINQDTQYF

SEQ ID NO: 145 - protein, 16 aa, Mus musculus
CASSLGTVSYNSPLYF

SEQ ID NO: 146 - protein, 14 aa, Mus musculus
CASSQETGNTEVFF

SEQ ID NO: 147 - protein, 14 aa, Mus musculus
CAWSLAGDNQAPLF

SEQ ID NO: 148 - protein, 15 aa, Mus musculus
CASSQEGTGTETLYF

SEQ ID NO: 149 - protein, 15 aa, Mus musculus
CASSSTGRNNQAPLF

SEQ ID NO: 150 - protein, 15 aa, Mus musculus
CASGDWGGATGQLYF

SEQ ID NO: 151 - protein, 15 aa, Mus musculus
CASGDAAGGTGQLYF

SEQ ID NO: 152 - protein, 12 aa, Mus musculus
CASSPTTYEQYF

SEQ ID NO: 153 - protein, 13 aa, Mus musculus
CASSLSGGYEQYF

SEQ ID NO: 154 - protein, 12 aa, Mus musculus
CASSPDSYEQYF

SEQ ID NO: 155 - protein, 14 aa, Mus musculus
CASSPGTNNQAPLF

SEQ ID NO: 156 - protein, 14 aa, Mus musculus
CASSPQGAGNTLYF

SEQ ID NO: 157 - protein, 14 aa, Mus musculus
CASSWTGSGNTLYF

SEQ ID NO: 158 - protein, 14 aa, Mus musculus
CASRLRDWGYEQYF

SEQ ID NO: 159 - protein, 15 aa, Mus musculus
CASSQDPGGGYEQYF

SEQ ID NO: 160 - protein, 13 aa, Mus musculus
CASSTGGVYEQYF

SEQ ID NO: 161 - protein, 12 aa, Mus musculus
CASSTSNSDYTF

SEQ ID NO: 162 - protein, 13 aa, Mus musculus
CTCSARDTYEQYF

SEQ ID NO: 163 - protein, 13 aa, Mus musculus
CASGGTGVYEQYF

SEQ ID NO: 164 - protein, 13 aa, Mus musculus
CASGTGGSYEQYF

SEQ ID NO: 165 - protein, 12 aa, Mus musculus
CASSDAIYEQYF

SEQ ID NO: 166 - protein, 15 aa, Mus musculus
CASSLAPDSGNTLYF

SEQ ID NO: 167 - protein, 14 aa, Mus musculus
CASSLRDGQDTQYF

SEQ ID NO: 168 - protein, 12 aa, Mus musculus
CASSSGDSDYTF

SEQ ID NO: 169 - protein, 15 aa, Mus musculus
CTCSARLGGYAEQFF

SEQ ID NO: 170 - protein, 11 aa, Mus musculus
CASSPPGQLYF

SEQ ID NO: 171 - protein, 14 aa, Mus musculus
CTCSAGGGAGEQYF

SEQ ID NO: 172 - protein, 13 aa, Mus musculus
CASRRQGNSDYTF

SEQ ID NO: 173 - protein, 11 aa, Mus musculus
CASSDGTEQYF

SEQ ID NO: 174 - protein, 14 aa, Mus musculus
CASSDQGSNERLFF

SEQ ID NO: 175 - protein, 14 aa, Mus musculus
CASSPTGGGNTLYF

SEQ ID NO: 176 - protein, 14 aa, Mus musculus
CASSRDNNYAEQFF

SEQ ID NO: 177 - protein, 11 aa, Mus musculus
CAWSRNSDYTF

SEQ ID NO: 178 - protein, 12 aa, Mus musculus
CASSFQQDTQYF

SEQ ID NO: 179 - protein, 13 aa, Mus musculus
CASSGDNAETLYF

SEQ ID NO: 180 - protein, 14 aa, Mus musculus
CASSLGLNQDTQYF

SEQ ID NO: 181 - protein, 15 aa, Mus musculus
CASGPGRISNERLFF

SEQ ID NO: 182 - protein, 14 aa, Mus musculus
CASSGTVNYAEQFF

SEQ ID NO: 183 - protein, 13 aa, Mus musculus
CASSLNSNSDYTF

SEQ ID NO: 184 - protein, 14 aa, Mus musculus
CASSPDSSAETLYF

SEQ ID NO: 185 - protein, 12 aa, Mus musculus
CASSPGQTEVFF

SEQ ID NO: 186 - protein, 14 aa, Mus musculus
CASSPTGSGNTLYF

SEQ ID NO: 187 - protein, 15 aa, Mus musculus
CASSQDGGGTGQLYF

SEQ ID NO: 188 - protein, 13 aa, Mus musculus
CASSQGYQDTQYF

SEQ ID NO: 189 - protein, 11 aa, Mus musculus
CASSFKDTQYF

SEQ ID NO: 190 - protein, 15 aa, Mus musculus
CASSIAGTGNERLFF

SEQ ID NO: 191 - protein, 14 aa, Mus musculus
CASSPDRGQNTLYF

SEQ ID NO: 192 - protein, 13 aa, Mus musculus
CASSWTGQDTQYF

SEQ ID NO: 193 - protein, 12 aa, Mus musculus
CASSYREDTQYF

SEQ ID NO: 194 - protein, 13 aa, Mus musculus
CASTGQANTEVFF

SEQ ID NO: 195 - protein, 14 aa, Mus musculus
CTCSADINQDTQYF

SEQ ID NO: 196 - protein, 14 aa, Mus musculus
CASGETGGNTEVFF

SEQ ID NO: 197 - protein, 14 aa, Mus musculus
CASGPGQSNTEVFF

SEQ ID NO: 198 - protein, 14 aa, Mus musculus
CASSGDNSAETLYF

SEQ ID NO: 199 - protein, 15 aa, Mus musculus
CASSLEAGGAETLYF

SEQ ID NO: 200 - protein, 11 aa, Mus musculus
CASSLQNTLYF

SEQ ID NO: 201 - protein, 11 aa, Mus musculus
CASSLRGEVFF

SEQ ID NO: 202 - protein, 14 aa, Mus musculus
CASSPGQGDTEVFF

SEQ ID NO: 203 - protein, 14 aa, Mus musculus
CTCSAGTGHTEVFF

SEQ ID NO: 204 - protein, 16 aa, Mus musculus
CASSPRTGGSAETLYF

SEQ ID NO: 205 - protein, 15 aa, Mus musculus
CASSLGTGVNQAPLF

SEQ ID NO: 206 - protein, 13 aa, Mus musculus
CTCSAGTKDTQYF

SEQ ID NO: 207 - protein, 12 aa, Mus musculus
CASSPTSYEQYF

SEQ ID NO: 208 - protein, 15 aa, Mus musculus
CASSLVGASAETLYF

SEQ ID NO: 209 - protein, 12 aa, Mus musculus
CGAREGEDTQYF

SEQ ID NO: 210 - protein, 14 aa, Mus musculus
CASSQDRDKYEQYF

SEQ ID NO: 211 - protein, 14 aa, Mus musculus
CASSRQGGDERLFF

SEQ ID NO: 212 - protein, 13 aa, Mus musculus
CASSLGGPYEQYF

SEQ ID NO: 213 - protein, 11 aa, Mus musculus
CASRNTGQLYF

SEQ ID NO: 214 - protein, 14 aa, Mus musculus
CASSRQGNYAEQFF

SEQ ID NO: 215 - protein, 15 aa, Mus musculus
CASSLGGANTGQLYF

SEQ ID NO: 216 - protein, 14 aa, Mus musculus
CASGDAGGRNTLYF

SEQ ID NO: 217 - protein, 13 aa, Mus musculus
CASGGGLQDTQYF

SEQ ID NO: 218 - protein, 14 aa, Mus musculus
CASSFDWGQDTQYF

SEQ ID NO: 219 - protein, 16 aa, Mus musculus
CASSLGLGVNQDTQYF

SEQ ID NO: 220 - protein, 14 aa, Mus musculus
CASSLGQSQNTLYF

SEQ ID NO: 221 - protein, 14 aa, Mus musculus
CASSLSGNQDTQYF

SEQ ID NO: 222 - protein, 13 aa, Mus musculus
CASSSGLQDTQYF

SEQ ID NO: 223 - protein, 14 aa, Mus musculus
CAWSPDRANTEVFF

SEQ ID NO: 224 - protein, 14 aa, Mus musculus
CASSLAGGNTEVFF

SEQ ID NO: 225 - protein, 14 aa, Mus musculus
CASSPGLGEDTQYF

SEQ ID NO: 226 - protein, 16 aa, Mus musculus
CASSQDGGASQNTLYF

SEQ ID NO: 227 - protein, 12 aa, Mus musculus
CAWSLDQDTQYF

SEQ ID NO: 228 - protein, 13 aa, Mus musculus
CASSEGSQDTQYF

SEQ ID NO: 229 - protein, 14 aa, Mus musculus
CASSSGTANTEVFF

SEQ ID NO: 230 - protein, 15 aa, Mus musculus
CASGDVGQGNERLFF

SEQ ID NO: 231 - protein, 14 aa, Mus musculus
CASSLPGTNERLFF

SEQ ID NO: 232 - protein, 14 aa, Mus musculus
CASSLSGNTGQLYF

SEQ ID NO: 233 - protein, 14 aa, Mus musculus
CTCSAGQNNQAPLF

SEQ ID NO: 234 - protein, 13 aa, Mus musculus
CASSLGGAREQYF

SEQ ID NO: 235 - protein, 14 aa, Mus musculus
CASSDLGGQDTQYF

SEQ ID NO: 236 - protein, 13 aa, Mus musculus
CASSQESQNTLYF

SEQ ID NO: 237 - protein, 14 aa, Mus musculus
CASSGTGGYAEQFF

SEQ ID NO: 238 - protein, 14 aa, Mus musculus
CASSQDNSQNTLYF

SEQ ID NO: 239 - protein, 14 aa, Mus musculus
CASSLGGAGNTLYF

SEQ ID NO: 240 - protein, 16 aa, Mus musculus
CASSQEGWGNQDTQYF

SEQ ID NO: 241 - protein, 17 aa, Mus musculus
CASSQDLWGSSQNTLYF

SEQ ID NO: 242 - protein, 12 aa, Mus musculus
CASSPTGEEQYF

SEQ ID NO: 243 - protein, 14 aa, Mus musculus
CTCSVTDSGNTLYF

SEQ ID NO: 244 - protein, 13 aa, Mus musculus
CASSLDNAETLYF

SEQ ID NO: 245 - protein, 14 aa, Mus musculus
CTCSAEGGRGEQYF

SEQ ID NO: 246 - protein, 13 aa, Mus musculus
CASSDWGEGEQYF

SEQ ID NO: 247 - protein, 13 aa, Mus musculus
CASSEDSGNTLYF

SEQ ID NO: 248 - protein, 12 aa, Mus musculus
CASSRGNSDYTF

SEQ ID NO: 249 - protein, 15 aa, Mus musculus
CASSSRDRGDSDYTF

SEQ ID NO: 250 - protein, 11 aa, Mus musculus
CASGGRYEQYF

SEQ ID NO: 251 - protein, 12 aa, Mus musculus
CASSDSGREQYF

SEQ ID NO: 252 - protein, 11 aa, Mus musculus
CASSLLGEQYF

SEQ ID NO: 253 - protein, 11 aa, Mus musculus
CASSRSYEQYF

SEQ ID NO: 254 - protein, 13 aa, Mus musculus
CAWSPRGNSDYTF

SEQ ID NO: 255 - protein, 15 aa, Mus musculus
CTCSADRGDYAEQFF

SEQ ID NO: 256 - protein, 16 aa, Mus musculus
CTCSAGTGGSNERLFF

SEQ ID NO: 257 - protein, 14 aa, Mus musculus
CASGDQGAGERLFF

SEQ ID NO: 258 - protein, 14 aa, Mus musculus
CASGDTGAGNTLYF

SEQ ID NO: 259 - protein, 12 aa, Mus musculus
CASGEGAYEQYF

SEQ ID NO: 260 - protein, 12 aa, Mus musculus
CASSATGGEQYF

SEQ ID NO: 261 - protein, 12 aa, Mus musculus
CASSDNYAEQFF

SEQ ID NO: 262 - protein, 14 aa, Mus musculus
CASSFGGANSDYTF

SEQ ID NO: 263 - protein, 14 aa, Mus musculus
CASSLKGSGNTLYF

SEQ ID NO: 264 - protein, 14 aa, Mus musculus
CASSLSLSNERLFF

SEQ ID NO: 265 - protein, 14 aa, Mus musculus
CASSPGQGAYEQYF

SEQ ID NO: 266 - protein, 14 aa, Mus musculus
CASSPLGGPYEQYF

SEQ ID NO: 267 - protein, 15 aa, Mus musculus
CASSQDWGLSYEQYF

SEQ ID NO: 268 - protein, 15 aa, Mus musculus
CASSQEGGGAYEQYF

SEQ ID NO: 269 - protein, 13 aa, Mus musculus
CASSRDSGNTLYF

SEQ ID NO: 270 - protein, 13 aa, Mus musculus
CASSRTGVYEQYF

SEQ ID NO: 271 - protein, 14 aa, Mus musculus
CASSDPGGTETLYF

SEQ ID NO: 272 - protein, 14 aa, Mus musculus
CASSDQGAYAEQFF

SEQ ID NO: 273 - protein, 13 aa, Mus musculus
CASSDRDTGQLYF

SEQ ID NO: 274 - protein, 12 aa, Mus musculus
CASSFTGDEQYF

SEQ ID NO: 275 - protein, 11 aa, Mus musculus
CASSMSYEQYF

SEQ ID NO: 276 - protein, 14 aa, Mus musculus
CASSPGDSGNTLYF

SEQ ID NO: 277 - protein, 15 aa, Mus musculus
CASSPGTGVNQAPLF

SEQ ID NO: 278 - protein, 14 aa, Mus musculus
CASSQDGQYAEQFF

SEQ ID NO: 279 - protein, 15 aa, Mus musculus
CASSQGLGVSYEQYF

SEQ ID NO: 280 - protein, 14 aa, Mus musculus
CASSRTGSAETLYF

SEQ ID NO: 281 - protein, 12 aa, Mus musculus
CASSSLSYEQYF

SEQ ID NO: 282 - protein, 13 aa, Mus musculus
CGAGTNNNQAPLF

SEQ ID NO: 283 - protein, 13 aa, Mus musculus
CTCSADLGSDYTF

SEQ ID NO: 284 - protein, 12 aa, Mus musculus
CASGVDSYEQYF

SEQ ID NO: 285 - protein, 14 aa, Mus musculus
CASSEGQGYAEQFF

SEQ ID NO: 286 - protein, 13 aa, Mus musculus
CASSFQGAYEQYF

SEQ ID NO: 287 - protein, 13 aa, Mus musculus
CASSGTTNSDYTF

SEQ ID NO: 288 - protein, 14 aa, Mus musculus
CASSLGGSNSDYTF

SEQ ID NO: 289 - protein, 14 aa, Mus musculus
CASSLSRNNQAPLF

SEQ ID NO: 290 - protein, 14 aa, Mus musculus
CASSMGRAGNTLYF

SEQ ID NO: 291 - protein, 14 aa, Mus musculus
CASSPDRNYAEQFF

SEQ ID NO: 292 - protein, 13 aa, Mus musculus
CASSPGQNERLFF

SEQ ID NO: 293 - protein, 13 aa, Mus musculus
CASSPGQSYEQYF

SEQ ID NO: 294 - protein, 14 aa, Mus musculus
CASSPTISNERLFF

SEQ ID NO: 295 - protein, 15 aa, Mus musculus
CASSQDGQGSYEQYF

SEQ ID NO: 296 - protein, 14 aa, Mus musculus
CASSQEQANSDYTF

SEQ ID NO: 297 - protein, 15 aa, Mus musculus
CASSQGHISNERLFF

SEQ ID NO: 298 - protein, 12 aa, Mus musculus
CASSYSQNTLYF

SEQ ID NO: 299 - protein, 14 aa, Mus musculus
CASTRDSSGNTLYF

SEQ ID NO: 300 - protein, 14 aa, Mus musculus
CAWSLPNSGNTLYF

SEQ ID NO: 301 - protein, 12 aa, Mus musculus
CASGDGRDEQYF

SEQ ID NO: 302 - protein, 15 aa, Mus musculus
CASGEGGNSGNTLYF

SEQ ID NO: 303 - protein, 13 aa, Mus musculus
CASGQGANERLFF

SEQ ID NO: 304 - protein, 12 aa, Mus musculus
CASRTTNSDYTF

SEQ ID NO: 305 - protein, 13 aa, Mus musculus
CASSDADRDEQYF

SEQ ID NO: 306 - protein, 14 aa, Mus musculus
CASSDARGRDTQYF

SEQ ID NO: 307 - protein, 14 aa, Mus musculus
CASSHRGGNQAPLF

SEQ ID NO: 308 - protein, 15 aa, Mus musculus
CASSLAGGGSYEQYF

SEQ ID NO: 309 - protein, 14 aa, Mus musculus
CASSLDISGNTLYF

SEQ ID NO: 310 - protein, 14 aa, Mus musculus
CASSLEGGDSDYTF

SEQ ID NO: 311 - protein, 12 aa, Mus musculus
CASSLGGPEQYF

SEQ ID NO: 312 - protein, 14 aa, Mus musculus
CASSLGGPYAEQFF

SEQ ID NO: 313 - protein, 13 aa, Mus musculus
CASSLTGGVEQYF

SEQ ID NO: 314 - protein, 15 aa, Mus musculus
CASSPGLGGSYEQYF

SEQ ID NO: 315 - protein, 15 aa, Mus musculus
CASSQDGVSGNTLYF

SEQ ID NO: 316 - protein, 13 aa, Mus musculus
CASSQEGGVEQYF

SEQ ID NO: 317 - protein, 15 aa, Mus musculus
CASSSGTGGGYEQYF

SEQ ID NO: 318 - protein, 13 aa, Mus musculus
CAWRQNSGNTLYF

SEQ ID NO: 319 - protein, 15 aa, Mus musculus
CAWSLGTNSGNTLYF

SEQ ID NO: 320 - protein, 12 aa, Mus musculus
CAWSLWGDEQYF

SEQ ID NO: 321 - protein, 13 aa, Mus musculus
CTCSAATNERLFF

SEQ ID NO: 322 - protein, 14 aa, Mus musculus
CASGARDNYAEQFF

SEQ ID NO: 323 - protein, 11 aa, Mus musculus
CASGAYAEQFF

SEQ ID NO: 324 - protein, 14 aa, Mus musculus
CASGDDTGGYEQYF

SEQ ID NO: 325 - protein, 8 aa, Mus musculus
CASGEQFF

SEQ ID NO: 326 - protein, 13 aa, Mus musculus
CASRDRNTGQLYF

SEQ ID NO: 327 - protein, 14 aa, Mus musculus
CASSDAVSQNTLYF

SEQ ID NO: 328 - protein, 14 aa, Mus musculus
CASSDLGDYAEQFF

SEQ ID NO: 329 - protein, 12 aa, Mus musculus
CASSFGGNTLYF

SEQ ID NO: 330 - protein, 13 aa, Mus musculus
CASSFQANSDYTF

SEQ ID NO: 331 - protein, 12 aa, Mus musculus
CASSFRNSDYTF

SEQ ID NO: 332 - protein, 13 aa, Mus musculus
CASSGGNYAEQFF

SEQ ID NO: 333 - protein, 15 aa, Mus musculus
CASSGGQGSAETLYF

SEQ ID NO: 334 - protein, 16 aa, Mus musculus
CASSHGLGGNYAEQFF

SEQ ID NO: 335 - protein, 13 aa, Mus musculus
CASSLAGRTEVFF

SEQ ID NO: 336 - protein, 14 aa, Mus musculus
CASSLDGGSYEQYF

SEQ ID NO: 337 - protein, 13 aa, Mus musculus
CASSLLGGREQYF

SEQ ID NO: 338 - protein, 14 aa, Mus musculus
CASSLLVNQDTQYF

SEQ ID NO: 339 - protein, 12 aa, Mus musculus
CASSLQGYEQYF

SEQ ID NO: 340 - protein, 14 aa, Mus musculus
CASSLRGSGNTLYF

SEQ ID NO: 341 - protein, 15 aa, Mus musculus
CASSLSVNSGNTLYF

SEQ ID NO: 342 - protein, 12 aa, Mus musculus
CASSLWGDEQYF

SEQ ID NO: 343 - protein, 14 aa, Mus musculus
CASSPTSSAETLYF

SEQ ID NO: 344 - protein, 13 aa, Mus musculus
CASSQDGQDTQYF

SEQ ID NO: 345 - protein, 13 aa, Mus musculus
CASSQEEGGEQYF

SEQ ID NO: 346 - protein, 13 aa, Mus musculus
CASSRDRGREQYF

SEQ ID NO: 347 - protein, 13 aa, Mus musculus
CASSRTTNSDYTF

SEQ ID NO: 348 - protein, 14 aa, Mus musculus
CASSSDRVGNTLYF

SEQ ID NO: 349 - protein, 15 aa, Mus musculus
CASSSGLGGENTLYF

SEQ ID NO: 350 - protein, 14 aa, Mus musculus
CASSSGTSNSDYTF

SEQ ID NO: 351 - protein, 12 aa, Mus musculus
CAWSLEGDTQYF

SEQ ID NO: 352 - protein, 15 aa, Mus musculus
CAWSLSGGARAEQFF

SEQ ID NO: 353 - protein, 13 aa, Mus musculus
CGARVGQNSDYTF

SEQ ID NO: 354 - protein, 13 aa, Mus musculus
CTCSAGGAPEQYF

SEQ ID NO: 355 - protein, 14 aa, Mus musculus
CASGDAGAEDTQYF

SEQ ID NO: 356 - protein, 16 aa, Mus musculus
CASGERLGVNQDTQYF

SEQ ID NO: 357 - protein, 14 aa, Mus musculus
CASGETGAQDTQYF

SEQ ID NO: 358 - protein, 13 aa, Mus musculus
CASRTSSAETLYF

SEQ ID NO: 359 - protein, 14 aa, Mus musculus
CASSDADIQDTQYF

SEQ ID NO: 360 - protein, 13 aa, Mus musculus
CASSDALNTEVFF

SEQ ID NO: 361 - protein, 11 aa, Mus musculus
CASSDRETLYF

SEQ ID NO: 362 - protein, 15 aa, Mus musculus
CASSDRGPNTGQLYF

SEQ ID NO: 363 - protein, 12 aa, Mus musculus
CASSERQNTLYF

SEQ ID NO: 364 - protein, 13 aa, Mus musculus
CASSGDSAETLYF

SEQ ID NO: 365 - protein, 14 aa, Mus musculus
CASSIGRNQDTQYF

SEQ ID NO: 366 - protein, 15 aa, Mus musculus
CASSLEGQNYAEQFF

SEQ ID NO: 367 - protein, 15 aa, Mus musculus
CASSLEGRNTGQLYF

SEQ ID NO: 368 - protein, 14 aa, Mus musculus
CASSLGFNQDTQYF

SEQ ID NO: 369 - protein, 14 aa, Mus musculus
CASSLGGAAETLYF

SEQ ID NO: 370 - protein, 14 aa, Mus musculus
CASSLGGGGAEQFF

SEQ ID NO: 371 - protein, 15 aa, Mus musculus
CASSLGTTNTGQLYF

SEQ ID NO: 372 - protein, 14 aa, Mus musculus
CASSLLGGRDTQYF

SEQ ID NO: 373 - protein, 13 aa, Mus musculus
CASSLLNQDTQYF

SEQ ID NO: 374 - protein, 14 aa, Mus musculus
CASSPDSSAETLYF

SEQ ID NO: 375 - protein, 15 aa, Mus musculus
CASSPDWGDTGQLYF

SEQ ID NO: 376 - protein, 13 aa, Mus musculus
CASSQAANTEVFF

SEQ ID NO: 377 - protein, 15 aa, Mus musculus
CASSQDHSSGNTLYF

SEQ ID NO: 378 - protein, 16 aa, Mus musculus
CASSQEGGRGAETLYF

SEQ ID NO: 379 - protein, 14 aa, Mus musculus
CASSQGRGAETLYF

SEQ ID NO: 380 - protein, 15 aa, Mus musculus
CASSQLGSSAETLYF

SEQ ID NO: 381 - protein, 14 aa, Mus musculus
CASSQPGANTEVFF

SEQ ID NO: 382 - protein, 14 aa, Mus musculus
CASSRDRNYAEQFF

SEQ ID NO: 383 - protein, 12 aa, Mus musculus
CASSRQGTEVFF

SEQ ID NO: 384 - protein, 10 aa, Mus musculus
CAWSLDTLYF

SEQ ID NO: 385 - protein, 12 aa, Mus musculus
CTCSAGDSPLYF

SEQ ID NO: 386 - protein, 15 aa, Mus musculus
CTCSAGQGADTEVFF

SEQ ID NO: 387 - protein, 13 aa, Mus musculus
CTCSAGVNSPLYF

SEQ ID NO: 388 - protein, 15 aa, Mus musculus
CASGDAGGTQDTQYF

SEQ ID NO: 389 - protein, 16 aa, Mus musculus
CASGDAGGVSQNTLYF

SEQ ID NO: 390 - protein, 14 aa, Mus musculus
CASGDAGRDTEVFF

SEQ ID NO: 391 - protein, 15 aa, Mus musculus
CASGDDWGGTGQLYF

SEQ ID NO: 392 - protein, 13 aa, Mus musculus
CASGDTGQNTLYF

SEQ ID NO: 393 - protein, 16 aa, Mus musculus
CASGEGTGGANTEVFF

SEQ ID NO: 394 - protein, 14 aa, Mus musculus
CASGQGASAETLYF

SEQ ID NO: 395 - protein, 13 aa, Mus musculus
CASRGTGDTEVFF

SEQ ID NO: 396 - protein, 14 aa, Mus musculus
CASSAGTTNTEVFF

SEQ ID NO: 397 - protein, 16 aa, Mus musculus
CASSDATGASQNTLYF

SEQ ID NO: 398 - protein, 14 aa, Mus musculus
CASSFTGGDTEVFF

SEQ ID NO: 399 - protein, 13 aa, Mus musculus
CASSHGQNTEVFF

SEQ ID NO: 400 - protein, 14 aa, Mus musculus
CASSKGQNTGQLYF

SEQ ID NO: 401 - protein, 13 aa, Mus musculus
CASSLASAETLYF

SEQ ID NO: 402 - protein, 14 aa, Mus musculus
CASSLDWGGREQYF

SEQ ID NO: 403 - protein, 12 aa, Mus musculus
CASSLEEDTQYF

SEQ ID NO: 404 - protein, 15 aa, Mus musculus
CASSLEGGSSYEQYF

SEQ ID NO: 405 - protein, 15 aa, Mus musculus
CASSLEGSSAETLYF

SEQ ID NO: 406 - protein, 13 aa, Mus musculus
CASSLGHNTEVFF

SEQ ID NO: 407 - protein, 14 aa, Mus musculus
CASSLGSYNSPLYF

SEQ ID NO: 408 - protein, 15 aa, Mus musculus
CASSLGTGSAETLYF

SEQ ID NO: 409 - protein, 13 aa, Mus musculus
CASSLGVQDTQYF

SEQ ID NO: 410 - protein, 16 aa, Mus musculus
CASSLRDWGNTGQLYF

SEQ ID NO: 411 - protein, 14 aa, Mus musculus
CASSLRGSAETLYF

SEQ ID NO: 412 - protein, 14 aa, Mus musculus
CASSLRVNQDTQYF

SEQ ID NO: 413 - protein, 15 aa, Mus musculus
CASSLSGQGNTEVFF

SEQ ID NO: 414 - protein, 14 aa, Mus musculus
CASSLVGDAETLYF

SEQ ID NO: 415 - protein, 14 aa, Mus musculus
CASSMGTTNTEVFF

SEQ ID NO: 416 - protein, 11 aa, Mus musculus
CASSPNTEVFF

SEQ ID NO: 417 - protein, 13 aa, Mus musculus
CASSPTGNTEVFF

SEQ ID NO: 418 - protein, 16 aa, Mus musculus
CASSQAGGASAETLYF

SEQ ID NO: 419 - protein, 14 aa, Mus musculus
CASSQEGGRNTLYF

SEQ ID NO: 420 - protein, 15 aa, Mus musculus
CASSQEGQGNSDYTF

SEQ ID NO: 421 - protein, 15 aa, Mus musculus
CASSQELGDYAEQFF

SEQ ID NO: 422 - protein, 13 aa, Mus musculus
CASSQGGGDTQYF

SEQ ID NO: 423 - protein, 12 aa, Mus musculus
CASSQRDTEVFF

SEQ ID NO: 424 - protein, 15 aa, Mus musculus
CASSRDWGGTGQLYF

SEQ ID NO: 425 - protein, 14 aa, Mus musculus
CASSRTGGDDTQYF

SEQ ID NO: 426 - protein, 14 aa, Mus musculus
CASSRTSSQNTLYF

SEQ ID NO: 427 - protein, 13 aa, Mus musculus
CASSVQGNTEVFF

SEQ ID NO: 428 - protein, 14 aa, Mus musculus
CAWSGQGANTEVFF

SEQ ID NO: 429 - protein, 15 aa, Mus musculus
CAWSLGDRGDERLFF

SEQ ID NO: 430 - protein, 14 aa, Mus musculus
CAWSLGGAEDTQYF

SEQ ID NO: 431 - protein, 13 aa, Mus musculus
CGARGTGGSDYTF

SEQ ID NO: 432 - protein, 11 aa, Mus musculus
CGASRNTEVFF

SEQ ID NO: 433 - protein, 13 aa, Mus musculus
CTCSADRGVEVFF

SEQ ID NO: 434 - protein, 14 aa, Mus musculus
CTCSAESSAETLYF

SEQ ID NO: 435 - protein, 13 aa, Mus musculus
CTCSAVGGDTQYF

SEQ ID NO: 436 - protein, 13 aa, Homo sapiens
CASSDGTTGELFF

SEQ ID NO: 437 - protein, 11 aa, Homo sapiens
CASSQHYEQYF

SEQ ID NO: 438 - protein, 15 aa, Homo sapiens
CASSFPRGSSYEQYF

SEQ ID NO: 439 - protein, 15 aa, Homo sapiens
CASSGTSGSTDTQYF

SEQ ID NO: 440 - protein, 15 aa, Homo sapiens
CASSDGTSGSNEQFF

SEQ ID NO: 441 - protein, 13 aa, Homo sapiens
CASSLSSNQPQHF

SEQ ID NO: 442 - protein, 13 aa, Homo sapiens
CASSLGGGETQYF

SEQ ID NO: 443 - protein, 15 aa, Homo sapiens
CASSPGPGNSYEQYF

SEQ ID NO: 444 - protein, 13 aa, Homo sapiens
CASSFTENTEAFF

SEQ ID NO: 445 - protein, 14 aa, Homo sapiens
CASSFGRGQETQYF

SEQ ID NO: 446 - protein, 12 aa, Homo sapiens
CASSQDGSPLHF

SEQ ID NO: 447 - protein, 13 aa, Homo sapiens
CASSPLSSYEQYF

SEQ ID NO: 448 - protein, 13 aa, Homo sapiens
CASSLRGNQPQHF

SEQ ID NO: 449 - protein, 14 aa, Homo sapiens
CASSLQGGNYGYTF

SEQ ID NO: 450 - protein, 15 aa, Homo sapiens
CASSIAARGNTEAFF

SEQ ID NO: 451 - protein, 12 aa, Homo sapiens
CSARQGDTEAFF

SEQ ID NO: 452 - protein, 12 aa, Homo sapiens
CASSLGGTEAFF

SEQ ID NO: 453 - protein, 14 aa, Homo sapiens
CASSLGRGGYGYTF

SEQ ID NO: 454 - protein, 15 aa, Homo sapiens
CASSLGTSASYEQYF

SEQ ID NO: 455 - protein, 13 aa, Homo sapiens
CASSMQGSTEAFF

SEQ ID NO: 456 - protein, 12 aa, Homo sapiens
CASSPTGDEQYF

SEQ ID NO: 457 - protein, 12 aa, Homo sapiens
CASRTVNQPQHF

SEQ ID NO: 458 - protein, 15 aa, Homo sapiens
CASSLAGTGGSGYTF

SEQ ID NO: 459 - protein, 14 aa, Homo sapiens
CASSLETNSYEQYF

SEQ ID NO: 460 - protein, 12 aa, Homo sapiens
CSAREGDTEAFF

SEQ ID NO: 461 - protein, 14 aa, Homo sapiens
CATIFQRGNQPQHF

SEQ ID NO: 462 - protein, 13 aa, Homo sapiens
CASSVTGGNEQFF

SEQ ID NO: 463 - protein, 12 aa, Homo sapiens
CSVGQDDYGYTF

SEQ ID NO: 464 - protein, 15 aa, Homo sapiens
CASSQAGTTYNEQFF

SEQ ID NO: 465 - protein, 13 aa, Homo sapiens
CASSIQGGTEAFF

SEQ ID NO: 466 - protein, 13 aa, Homo sapiens
CASRRQGNTEAFF

SEQ ID NO: 467 - protein, 14 aa, Homo sapiens
CASSRDPGRTEAFF

SEQ ID NO: 468 - protein, 13 aa, Homo sapiens
CASSLEGDQPQHF

SEQ ID NO: 469 - protein, 13 aa, Homo sapiens
CASSSRSSYEQYF

SEQ ID NO: 470 - protein, 11 aa, Homo sapiens
CSARERYEQYF

SEQ ID NO: 471 - protein, 14 aa, Homo sapiens
CAISGTSGTYEQYF

SEQ ID NO: 472 - protein, 14 aa, Homo sapiens
CASSWDGSNQPQHF

SEQ ID NO: 473 - protein, 13 aa, Homo sapiens
CASSTQGNTEAFF

SEQ ID NO: 474 - protein, 14 aa, Homo sapiens
CASSYGQENQPQHF

SEQ ID NO: 475 - protein, 14 aa, Homo sapiens
CASSGNRGGQPQHF

SEQ ID NO: 476 - protein, 14 aa, Homo sapiens
CASSVETGAETQYF

SEQ ID NO: 477 - protein, 18 aa, Homo sapiens
CASSQVLAGGSSYNEQFF

SEQ ID NO: 478 - protein, 15 aa, Homo sapiens
CASSLGTASTDTQYF

SEQ ID NO: 479 - protein, 13 aa, Homo sapiens
CASSSQGGTEAFF

SEQ ID NO: 480 - protein, 13 aa, Homo sapiens
CASRRGVNQPQHF

SEQ ID NO: 481 - protein, 13 aa, Homo sapiens
CASSYEGPYEQYF

SEQ ID NO: 482 - protein, 13 aa, Homo sapiens
CASSFEGAYEQYF

SEQ ID NO: 483 - protein, 11 aa, Homo sapiens
CASSSTGELFF

SEQ ID NO: 484 - protein, 11 aa, Homo sapiens
CASSLVGEQYF

SEQ ID NO: 485 - protein, 13 aa, Homo sapiens
CASSRDSNQPQHF

SEQ ID NO: 486 - protein, 13 aa, Homo sapiens
CASSYGGRQPQHF

SEQ ID NO: 487 - protein, 14 aa, Homo sapiens
CASSDSSGANVLTF

SEQ ID NO: 488 - protein, 12 aa, Homo sapiens
CASSLEGYEQYF

SEQ ID NO: 489 - protein, 11 aa, Homo sapiens
CASSLRYEQYF

SEQ ID NO: 490 - protein, 13 aa, Homo sapiens
CASSRGDNQPQHF

SEQ ID NO: 491 - protein, 14 aa, Homo sapiens
CASSLRRGTDTQYF

SEQ ID NO: 492 - protein, 13 aa, Homo sapiens
CASSQGGEETQYF

SEQ ID NO: 493 - protein, 13 aa, Homo sapiens
CASSPGTSYEQYF

SEQ ID NO: 494 - protein, 13 aa, Homo sapiens
CASSSTSTDTQYF

SEQ ID NO: 495 - protein, 13 aa, Homo sapiens
CASSLEYGYEQYF

SEQ ID NO: 496 - protein, 10 aa, Homo sapiens
CSVLDNGYTF

SEQ ID NO: 497 - protein, 13 aa, Homo sapiens
CASSEGQSYEQYF

SEQ ID NO: 498 - protein, 15 aa, Homo sapiens
CASSFTGSPGQEQYF

SEQ ID NO: 499 - protein, 13 aa, Homo sapiens
CSARDRTGNGYTF

SEQ ID NO: 500 - protein, 13 aa, Homo sapiens
CSARQDSNQPQHF

SEQ ID NO: 501 - protein, 15 aa, Homo sapiens
CASSQDRAGGTEAFF

SEQ ID NO: 502 - protein, 13 aa, Homo sapiens
CASSVGAGTEAFF

SEQ ID NO: 503 - protein, 16 aa, Homo sapiens
CASSQGDQGAKNIQYF

SEQ ID NO: 504 - protein, 13 aa, Homo sapiens
CASSFEGPYEQYF

SEQ ID NO: 505 - protein, 15 aa, Homo sapiens
CASSDSTSGSNEQFF

SEQ ID NO: 506 - protein, 15 aa, Homo sapiens
CASSEGQVWPGELFF

SEQ ID NO: 507 - protein, 13 aa, Homo sapiens
CASSDSDTGELFF

SEQ ID NO: 508 - protein, 16 aa, Homo sapiens
CASSLEGDLSGNTIYF

SEQ ID NO: 509 - protein, 14 aa, Homo sapiens
CASSYSSGANVLTF

SEQ ID NO: 510 - protein, 14 aa, Homo sapiens
CASSLVVQPYEQYF

SEQ ID NO: 511 - protein, 11 aa, Homo sapiens
CSASGRETQYF

SEQ ID NO: 512 - protein, 11 aa, Homo sapiens
CASSGVYGYTF

SEQ ID NO: 513 - protein, 13 aa, Homo sapiens
CASSPQGGYGYTF

SEQ ID NO: 514 - protein, 14 aa, Homo sapiens
CASSYSGQGFEQYF

SEQ ID NO: 515 - protein, 12 aa, Homo sapiens
CASSLDADLQYF

SEQ ID NO: 516 - protein, 14 aa, Homo sapiens
CASSLQGMNTEAFF

SEQ ID NO: 517 - protein, 13 aa, Homo sapiens
CSARGGIPYEQYF

SEQ ID NO: 518 - protein, 14 aa, Homo sapiens
CATSDRTGGNEQYF

SEQ ID NO: 519 - protein, 14 aa, Homo sapiens
CAWSRQGGNQPQHF

SEQ ID NO: 520 - protein, 14 aa, Homo sapiens
CASSIEGARTEAFF

SEQ ID NO: 521 - protein, 13 aa, Homo sapiens
CASSLDRSYEQYF

SEQ ID NO: 522 - protein, 17 aa, Homo sapiens
CASSVGSGGSSTDTQYF

SEQ ID NO: 523 - protein, 13 aa, Homo sapiens
CASSVTGGYEQYF

SEQ ID NO: 524 - protein, 14 aa, Homo sapiens
CASSIRGGNTEAFF

SEQ ID NO: 525 - protein, 15 aa, Homo sapiens
CASSAWRGGFHEQYF

SEQ ID NO: 526 - protein, 14 aa, Homo sapiens
CASSEGQGTYEQYF

SEQ ID NO: 527 - protein, 13 aa, Homo sapiens
CATSDSRITEQFF

SEQ ID NO: 528 - protein, 15 aa, Homo sapiens
CASSLEAARNQPQHF

SEQ ID NO: 529 - protein, 13 aa, Homo sapiens
CASRPGQGQPQHF

SEQ ID NO: 530 - protein, 13 aa, Homo sapiens
CASSLQGNTEAFF

SEQ ID NO: 531 - protein, 11 aa, Homo sapiens
CASSLGETQYF

SEQ ID NO: 532 - protein, 12 aa, Homo sapiens
CASSLRGYGYTF

SEQ ID NO: 533 - protein, 13 aa, Homo sapiens
CASSVTTGYEQYF

SEQ ID NO: 534 - protein, 15 aa, Homo sapiens
CSAGLAGGTPDTQYF

SEQ ID NO: 535 - protein, 14 aa, Homo sapiens
CASSLRGSSYEQYF

SEQ ID NO: 536 - protein, 13 aa, Homo sapiens
CASSLEGPYEQYF

SEQ ID NO: 537 - protein, 14 aa, Homo sapiens
CASSFGRGNTEAFF

SEQ ID NO: 538 - protein, 13 aa, Homo sapiens
CAWSLKGDSPLHF

SEQ ID NO: 539 - protein, 13 aa, Homo sapiens
CASSYSDTYEQYF

SEQ ID NO: 540 - protein, 13 aa, Homo sapiens
CASSQGGNTEAFF

SEQ ID NO: 541 - protein, 14 aa, Homo sapiens
CASSYRDSNQPQHF

SEQ ID NO: 542 - protein, 15 aa, Homo sapiens
CASSLWGTSTDTQYF

SEQ ID NO: 543 - protein, 15 aa, Homo sapiens
CASSLGQTYNSPLHF

SEQ ID NO: 544 - protein, 14 aa, Homo sapiens
CATSEGQGAVGYTF

SEQ ID NO: 545 - protein, 14 aa, Homo sapiens
CASSPEGQAAGYTF

SEQ ID NO: 546 - protein, 13 aa, Homo sapiens
CASSLTSTDTQYF

SEQ ID NO: 547 - protein, 15 aa, Homo sapiens
CASSLAAGSGNTIYF

SEQ ID NO: 548 - protein, 13 aa, Homo sapiens
CASSIRSAYEQYF

SEQ ID NO: 549 - protein, 13 aa, Homo sapiens
CASSLTGGYEQYF

SEQ ID NO: 550 - protein, 14 aa, Homo sapiens
CASSYSTSGYEQYF

SEQ ID NO: 551 - protein, 14 aa, Homo sapiens
CASSLASGWYEQYF

SEQ ID NO: 552 - protein, 14 aa, Homo sapiens
CASSRGTGDTEAFF

SEQ ID NO: 553 - protein, 14 aa, Homo sapiens
CASSDGQGADTQYF

SEQ ID NO: 554 - protein, 14 aa, Homo sapiens
CATSDGQGEVGYTF

SEQ ID NO: 555 - protein, 14 aa, Homo sapiens
CASSATGGNQPQHF

SEQ ID NO: 556 - protein, 13 aa, Homo sapiens
CASSIQGNTEAFF

SEQ ID NO: 557 - protein, 12 aa, Homo sapiens
CSASRESDTQYF

SEQ ID NO: 558 - protein, 14 aa, Homo sapiens
CASSQGDRGYGYTF

SEQ ID NO: 559 - protein, 14 aa, Homo sapiens
CASSPTGSSYEQYF

SEQ ID NO: 560 - protein, 14 aa, Homo sapiens
CASSVDSLNYGYTF

SEQ ID NO: 561 - protein, 13 aa, Homo sapiens
CASSVRSSYEQYF

SEQ ID NO: 562 - protein, 14 aa, Homo sapiens
CASSLETNTGELFF

SEQ ID NO: 563 - protein, 13 aa, Homo sapiens
CASSLEGGYEQYF

SEQ ID NO: 564 - protein, 14 aa, Homo sapiens
CSARLAGGQETQYF

SEQ ID NO: 565 - protein, 14 aa, Homo sapiens
CATSEGQGDVGYTF

SEQ ID NO: 566 - protein, 11 aa, Homo sapiens
CASSHSYEQYF

SEQ ID NO: 567 - protein, 13 aa, Homo sapiens
CASSLPSAGGYTF

SEQ ID NO: 568 - protein, 13 aa, Homo sapiens
CASSDSNTGELFF

SEQ ID NO: 569 - protein, 14 aa, Homo sapiens
CASSPGLAGDEQYF

SEQ ID NO: 570 - protein, 13 aa, Homo sapiens
CASSVGDNQPQHF

SEQ ID NO: 571 - protein, 13 aa, Homo sapiens
CASSLSSNQPQHF

SEQ ID NO: 572 - protein, 11 aa, Homo sapiens
CASSPGYEQYF

SEQ ID NO: 573 - protein, 14 aa, Homo sapiens
CSARGRAYNQPQHF

SEQ ID NO: 574 - protein, 14 aa, Homo sapiens
CASSPGQGRYEQYF

SEQ ID NO: 575 - protein, 14 aa, Homo sapiens
CASSQDGFNQPQHF

SEQ ID NO: 576 - protein, 13 aa, Homo sapiens
CASSLETNTEAFF

SEQ ID NO: 577 - protein, 12 aa, Homo sapiens
CASSFRNQPQHF

SEQ ID NO: 578 - protein, 13 aa, Homo sapiens
CASSQAGGTEAFF

SEQ ID NO: 579 - protein, 15 aa, Homo sapiens
CASSPGQVNTGELFF

SEQ ID NO: 580 - protein, 15 aa, Homo sapiens
CASSESTGYNQPQHF

SEQ ID NO: 581 - protein, 13 aa, Homo sapiens
CASSQQGADTQYF

SEQ ID NO: 582 - protein, 13 aa, Homo sapiens
CASSATGSYGYTF

SEQ ID NO: 583 - protein, 14 aa, Homo sapiens
CASSLGRGPYGYTF

SEQ ID NO: 584 - protein, 16 aa, Homo sapiens
CASSLRGGERGNTIYF

SEQ ID NO: 585 - protein, 13 aa, Homo sapiens
CASSPEGPYEQYF

SEQ ID NO: 586 - protein, 14 aa, Homo sapiens
CASSHQPGDYEQYF

SEQ ID NO: 587 - protein, 13 aa, Homo sapiens
CASSSGTYNEQFF

SEQ ID NO: 588 - protein, 16 aa, Homo sapiens
CASSESPGNSNQPQHF

SEQ ID NO: 589 - protein, 13 aa, Homo sapiens
CASSGGRDYGYTF

SEQ ID NO: 590 - protein, 14 aa, Homo sapiens
CSVGTGGTNEKLFF

SEQ ID NO: 591 - protein, 11 aa, Homo sapiens
CASSRTGELFF

SEQ ID NO: 592 - protein, 16 aa, Homo sapiens
CASSPPPGTGADTQYF

SEQ ID NO: 593 - protein, 15 aa, Homo sapiens
CATSRDSSGANVLTF

SEQ ID NO: 594 - protein, 14 aa, Homo sapiens
CASSYSRQGDGYTF

SEQ ID NO: 595 - protein, 16 aa, Homo sapiens
CASSQGWSSGGYEQYF

SEQ ID NO: 596 - protein, 13 aa, Homo sapiens
CASSLVDSVGYTF

SEQ ID NO: 597 - protein, 15 aa, Homo sapiens
CASSSPRGSSYEQYF

SEQ ID NO: 598 - protein, 13 aa, Homo sapiens
CASNQQGSTEAFF

SEQ ID NO: 599 - protein, 13 aa, Homo sapiens
CAWSVMGNYGYTF

SEQ ID NO: 600 - protein, 15 aa, Homo sapiens
CASSQAGTGVYEQYF

SEQ ID NO: 601 - protein, 15 aa, Homo sapiens
CASSEGTSGSYEQYF

SEQ ID NO: 602 - protein, 11 aa, Homo sapiens
CASSLSYEQYF

SEQ ID NO: 603 - protein, 12 aa, Homo sapiens
CASSYSTGEAFF

SEQ ID NO: 604 - protein, 14 aa, Homo sapiens
CASSLTGAYNEQFF

SEQ ID NO: 605 - protein, 14 aa, Homo sapiens
CASSLGQGNYGYTF

SEQ ID NO: 606 - protein, 13 aa, Homo sapiens
CASSGDGNYGYTF

SEQ ID NO: 607 - protein, 14 aa, Homo sapiens
CASSDNSGANVLTF

SEQ ID NO: 608 - protein, 13 aa, Homo sapiens
CASSLAGGTEAFF

SEQ ID NO: 609 - protein, 13 aa, Homo sapiens
CASSPAGGTEAFF

SEQ ID NO: 610 - protein, 12 aa, Homo sapiens
CASSIGTDTQYF

SEQ ID NO: 611 - protein, 12 aa, Homo sapiens
CASSLGGGEAFF

SEQ ID NO: 612 - protein, 12 aa, Homo sapiens
CASSLEGYGYTF

SEQ ID NO: 613 - protein, 14 aa, Homo sapiens
CASSLEGGNTEAFF

SEQ ID NO: 614 - protein, 14 aa, Homo sapiens
CAWSGQGGNQPQHF

SEQ ID NO: 615 - protein, 13 aa, Homo sapiens
CASRAGGYYGYTF

SEQ ID NO: 616 - protein, 14 aa, Homo sapiens
CAWRGQGGNQPQHF

SEQ ID NO: 617 - protein, 13 aa, Homo sapiens
CASRHRDSYEQYF

SEQ ID NO: 618 - protein, 13 aa, Homo sapiens
CASSYSERSEQFF

SEQ ID NO: 619 - protein, 13 aa, Homo sapiens
CASSIQGSTEAFF

SEQ ID NO: 620 - protein, 12 aa, Homo sapiens
CASRQGSYEQYF

SEQ ID NO: 621 - protein, 14 aa, Homo sapiens
CASGRDISTDTQYF

SEQ ID NO: 622 - protein, 11 aa, Homo sapiens
CSARDGYEQYF

SEQ ID NO: 623 - protein, 15 aa, Homo sapiens
CASSRAARGNTEAFF

SEQ ID NO: 624 - protein, 13 aa, Homo sapiens
CASSTGDNQPQHF

SEQ ID NO: 625 - protein, 15 aa, Homo sapiens
CASSQLVGPYSPLHF

SEQ ID NO: 626 - protein, 13 aa, Homo sapiens
CASSDRGTGELFF

SEQ ID NO: 627 - protein, 14 aa, Homo sapiens
CASSPGTANTEAFF

SEQ ID NO: 628 - protein, 15 aa, Homo sapiens
CASSPGTANTGELFF

SEQ ID NO: 629 - protein, 13 aa, Homo sapiens
CASSLGQAYEQYF

SEQ ID NO: 630 - protein, 17 aa, Homo sapiens
CASSYSKTGGSNQPQHF

SEQ ID NO: 631 - protein, 12 aa, Homo sapiens
CASSVENYGYTF

SEQ ID NO: 632 - protein, 15 aa, Homo sapiens
CASRVQGLGNQPQHF

SEQ ID NO: 633 - protein, 14 aa, Homo sapiens
CASSQDKGGTEAFF

SEQ ID NO: 634 - protein, 8 aa, Homo sapiens
CASSGDTF

SEQ ID NO: 635 - protein, 12 aa, Homo sapiens
CASSPTGPEQYF

SEQ ID NO: 636 - protein, 15 aa, Homo sapiens
CASSPGQGVNYGYTF

SEQ ID NO: 637 - protein, 15 aa, Homo sapiens
CSARDDRGSYNEQFF

SEQ ID NO: 638 - protein, 15 aa, Homo sapiens
CASSIIGASNQPQHF

SEQ ID NO: 639 - protein, 13 aa, Homo sapiens
CASSLSGNSPLHF

SEQ ID NO: 640 - protein, 13 aa, Homo sapiens
CASSFETYNEQFF

SEQ ID NO: 641 - protein, 13 aa, Homo sapiens
CASSLAGHQPQHF

SEQ ID NO: 642 - protein, 14 aa, Homo sapiens
CASSFETNTGELFF

SEQ ID NO: 643 - protein, 11 aa, Homo sapiens
CASSVGGGYTF

SEQ ID NO: 644 - protein, 14 aa, Homo sapiens
CASSVGWGNTEAFF

SEQ ID NO: 645 - protein, 13 aa, Homo sapiens
CASSPQRNTEAFF

SEQ ID NO: 646 - protein, 14 aa, Homo sapiens
CASSQDRTGPEQYF

SEQ ID NO: 647 - protein, 14 aa, Homo sapiens
CASSESQGNTEAFF

SEQ ID NO: 648 - protein, 15 aa, Homo sapiens
CASSDGTSGYNEQFF

SEQ ID NO: 649 - protein, 14 aa, Homo sapiens
CASSPGQGGQPQHF

SEQ ID NO: 650 - protein, 8 aa, Homo sapiens
CAGGGQYF

SEQ ID NO: 651 - protein, 13 aa, Homo sapiens
CASSIRSSYEQYF

SEQ ID NO: 652 - protein, 13 aa, Homo sapiens
CASSDRDTGELFF

SEQ ID NO: 653 - protein, 14 aa, Homo sapiens
CASSLDRVGTEAFF

SEQ ID NO: 654 - protein, 14 aa, Homo sapiens
CASSQEGRNTEAFF

SEQ ID NO: 655 - protein, 15 aa, Homo sapiens
CASSIAGIYNSPLHF

SEQ ID NO: 656 - protein, 15 aa, Homo sapiens
CASSPGTASGNTIYF

SEQ ID NO: 657 - protein, 14 aa, Homo sapiens
CATSEGQGETEAFF

SEQ ID NO: 658 - protein, 14 aa, Homo sapiens
CASSLRGSSYEQYF

SEQ ID NO: 659 - protein, 14 aa, Homo sapiens
CASSLVVSPYEQYF

SEQ ID NO: 660 - protein, 13 aa, Homo sapiens
CASGTGDNQPQHF

SEQ ID NO: 661 - protein, 14 aa, Homo sapiens
CASSLQGANYEQYF

SEQ ID NO: 662 - protein, 11 aa, Homo sapiens
CASSLGRTIYF

SEQ ID NO: 663 - protein, 13 aa, Homo sapiens
CASSIQGDTEAFF

SEQ ID NO: 664 - protein, 14 aa, Homo sapiens
CASSYGTNSYEQYF

SEQ ID NO: 665 - protein, 15 aa, Homo sapiens
CASSVTPGQGHEQYF

SEQ ID NO: 666 - protein, 14 aa, Homo sapiens
CASSLGRGNTEAFF

SEQ ID NO: 667 - protein, 12 aa, Homo sapiens
CSARDGNQPQHF

SEQ ID NO: 668 - protein, 14 aa, Homo sapiens
CASSLDSPNYGYTF

SEQ ID NO: 669 - protein, 15 aa, Homo sapiens
CASSPRGRGNQPQHF

SEQ ID NO: 670 - protein, 14 aa, Homo sapiens
CASSSGQPNTEAFF

SEQ ID NO: 671 - protein, 14 aa, Homo sapiens
CASSLQGAWGELFF

SEQ ID NO: 672 - protein, 14 aa, Homo sapiens
CASSFLAGAREQYF

SEQ ID NO: 673 - protein, 11 aa, Homo sapiens
CASGLFHEQYF

SEQ ID NO: 674 - protein, 17 aa, Homo sapiens
CATSDLVGTGGTGELFF

SEQ ID NO: 675 - protein, 9 aa, Artificial Sequence
  Synthetic construct
RLYDYFTRV

SEQ ID NO: 676 - protein, 9 aa, Artificial Sequence
  Synthetic construct
ILDDNLYKV

SEQ ID NO: 677 - protein, 9 aa, Artificial Sequence
  Synthetic construct
ALDEKLFLI

SEQ ID NO: 678 - protein, 9 aa, Artificial Sequence
  Synthetic construct
GLFDFVNFV

SEQ ID NO: 679 - protein, 9 aa, Artificial Sequence
  Sythetic construct
KVDDTFYYV

SEQ ID NO: 680 - protein, 9 aa, Artificial Sequence
  Synthetic constuct
RVYEALYYV

SEQ ID NO: 681 - protein, 9 aa, Artificial Sequence
  Synthetic construct
KIDDMIEEV

SEQ ID NO: 682 - protein, 9 aa, Artificial Sequence
  Synthetic construct
SLSNLDFRL

SEQ ID NO: 683 - protein, 9 aa, Artificial Sequence
  Synthetic construct
ILSDENYLL